(19) United States
(12) Patent Application Publication
(10) Pub. No.: US 2026/0306676 A1
(43) Pub. Date: Oct. 8, 2026

(54) FLUORESCEIN-LABELED NERVE TARGETING PEPTIDE FORMULATIONS AND RELATED METHODS

(71) Applicant: ALUME BIOSCIENCES, INC., San Diego, CA (US)

(72) Inventor: Quyen T. NGUYEN, San Diego, CA (US)

(21) Appl. No.: 19/483,211

(22) PCT Filed: May 8, 2024
(86) PCT No.: PCT/US2024/028401

§ 371 Date: Nov. 10, 2025

Related U.S. Application Data
(60) Provisional application No. 63/501,593, filed on May 11, 2023.

(51) Int. Cl. A61K 49/00; A61K 9/00; A61K 9/19; A61K 47/18; A61K 47/26

(52) U.S. Cl. CPC A61K 49/0056; A61K 9/0019; A61K 9/19; A61K 47/183; A61K 47/26; A61K 49/0041

(57) ABSTRACT

Disclosed herein are pharmaceutical compositions containing (a) a fluores-cein-labeled nerve targeting peptide of formula (I): or pharmaceutically acceptable salt, solvate, hydrate, isomer, tautomer, racemate, or isotope thereof; (b) an additive comprising a pH-adjusting agent, a buffering agent, a bulking agent, a tonicity-adjusting agent, a preservative, or any combination thereof; and (c) an aqueous vehicle, wherein a pH of the composition ranges from about 7 to about 9, and a concentration of the peptide in the aqueous composition ranges from about 25 mg/mL to about 200 mg/mL, based on a total volume of the aqueous composition. Also disclosed are methods for preparing and using the pharmaceutical compositions described herein.

### FIELD

Disclosed herein are fluorescein-labeled nerve targeting peptide formulations and methods related thereto, including their use in intraoperative nerve visualization and other targeted surgical and medical applications.

### BACKGROUND

Preserving human neurons and human nerves is an important goal of surgical procedures; for example, accidental transection of nerves can lead to significant morbidity, such as chronic pain and loss of function. Nerves are typically identified by their elongated whitish appearance and relationship to nearby structures or by electrophysiological studies if they are motor nerves (sensory and autonomic nerves cannot be monitored with electrophysiology). However, such nerve identification during surgery can be challenging especially in more complex surgical settings. Nerve structures can be obscured or distorted in complex surgical settings, such as those involving trauma, tumors, inflammation, prior surgery, prior radiation treatment or infection. Nerve visibility can be further restricted in minimally invasive procedures, such an endoscopy, which rely on small incisions with limited access. Indeed, inadvertent nerve damage is a leading complication associated with many surgeries using both open and minimal access procedures.

These observations highlight the need for methods and tools to identify neurons or nerves reliably and efficiently. Electromyography (EMG) offers one approach to motor nerve identification prior to direct exposure during surgery or confirmation of neuron or nerve identity in instances of uncertainty. A disadvantage of this technique, however, is the lack of visual feedback to the operating surgeon. Even if a nerve has been identified in one location, either through accidental or purposeful stimulation, there is no visual guidance to the operating surgeon as to how far away from the stimulation site the nerve lies or the direction of travel the nerve takes away from the stimulation site. Furthermore, EMG only traces motor nerves, not sensory or autonomic nerves. EMG can fail if neuron or nerve conduction or neuromuscular transmission is temporarily blocked anywhere distal to the recording site. Such blockade easily occurs due to neuron or nerve compression, trauma, local anesthetics, or neuromuscular blockers.

Other approaches include neuron or nerve labeling, which primarily depend on retrograde or anterograde tracing of individually identified axonal tracts via the use of fluorescent dyes. However, methods of labeling neuron or nerves by locally applied fluorescent tracers can be limited. First, depending on where the dye has been injected, this technique can label only one neuron or nerve fiber tract at a time. Second, this technique results in only limited labeling of fluorescent dyes along the axonal tracts because retrograde axonal tracers typically accumulate in the neural cell body. Third, retrograde transport is relatively slow (on the order of millimeters per day) and therefore takes a long time to label human neuron or nerves, which are often longer than a meter, such as in the case of the sciatic neuron or nerve and its arborizations. Fourth, the application of fluorescent dyes to innervation targets, such as direct intramuscular injections to label a motor neuron or nerves, is typically messy with a variable amount of the tracer dye remaining at the injection site. As dissection of neurons or nerves depends on accurate visualization of adjacent structures prior to encountering them, a surgical site that is contaminated with fluorescent dyes would not be desirable. Finally, the direct injection of the fluorescent dye itself may be damaging to the target organs or neuron or nerve of interest, either by mechanical damage or by the very high local concentration of dye and vehicle at the injection site.

Despite these difficulties, a promising approach has been disclosed in WO2019/028281, the entire contents of which are incorporating herein by reference, which provides optimized peptides for targeting human nerves and their use in image guided surgery, diagnostics and therapeutic delivery. In particular, a fluorescein-labeled nerve targeting peptide known as “Bevonescein (ALM-488)” (i.e., SEQ ID NO:104 of WO2019/028281) (also referred herein as the compound or peptide of formula (I)) has progressed into clinical trials (Phase 1/2 and phase 3) in order to evaluate the safety, tolerability, and efficacy of this compound for intravenous (IV) administration to patients undergoing head and neck surgery. FIG. 1 shows the structure of Bevonescein (ALM-488).

However, formulating Bevonescein (ALM-488) in a manner suitable for use in these clinical trials has proved challenging, and there remains a need to discover suitable formulations for IV administration. To this end, the present disclosure has overcome these challenges and provides formulations of Bevonescein (ALM-488) that are suitable for the manufacture, storage, reconstitution and IV infusion into patients. These and other aspects of the invention will become evident up reference to the following disclosure.

### BRIEF SUMMARY

In one aspect, the present disclosure provides aqueous pharmaceutical compositions comprising: (a) a fluorescein-labeled nerve targeting peptide of formula (I):

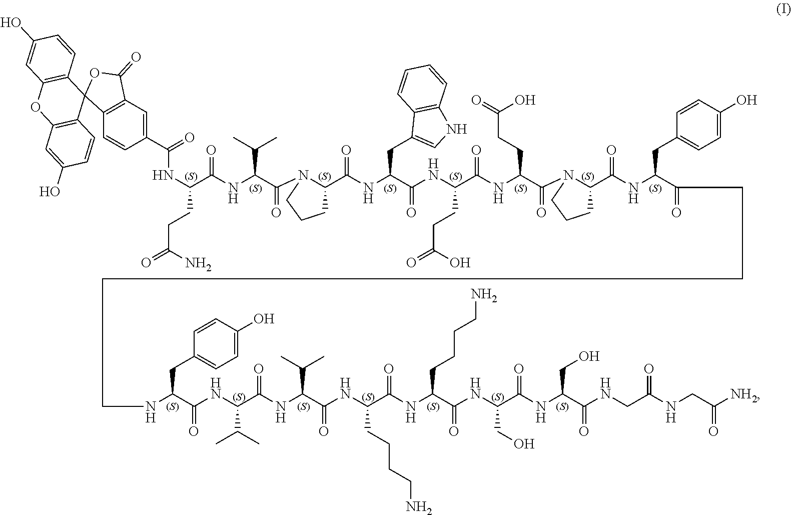

or pharmaceutically acceptable salt, solvate, hydrate, isomer, tautomer, racemate, or isotope thereof; (b) an additive comprising a pH-adjusting agent, a buffering agent, a bulking agent, a tonicity-adjusting agent, a preservative, or any combination thereof; and (c) an aqueous vehicle, wherein a pH of the composition ranges from about 7 to about 9, and a concentration of the peptide in the aqueous composition ranges from about 25 mg/mL to about 200 mg/mL, based on a total volume of the aqueous composition.

Other aspects relate to lyophilized pharmaceutical compositions and methods of forming lyophilized pharmaceutical compositions from aqueous pharmaceutical compositions of the present disclosure.

Other aspects relate to reconstituted aqueous pharmaceutical compositions and methods of forming reconstituted aqueous pharmaceutical compositions from lyophilized pharmaceutical compositions of the present disclosure.

Other aspects relate to methods of identifying neurons or nerves by contacting the neurons or nerves with a composition of the present disclosure.

Other aspects relate to methods of delivering at least one drug to a neuron or nerve by contacting the neuron or nerve with a composition of the present disclosure.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is the structure of Bevonescein (ALM-488);

FIG. 2 is an HPLC chromatogram of a 0.25 mg/mL solution of ALM-488 as an HCl salt in water;

FIG. 3 is a magnified view of the main peaks in the HPLC chromatogram of FIG. 2;

FIG. 4 is a flow diagram of the synthetic route used to produce ALM-488;

FIG. 5 is a thermogravimetric weight loss curve for a chloride salt of ALM-488;

FIG. 6 is a plot showing the linear relationship between peak area and the amount of ALM-488 in the HPLC method of Table 3;

FIG. 7 is a pH titration curve showing pH as a function of added NaOH and how solubility of an HCl salt of ALM-488 is affected by pH;

FIG. 8 is an overlay of pH titration curves showing pH as a function of added NaOH and how solubility of HCl and acetate salts of ALM-488 are affected by pH;

FIG. 9 is an overlay of magnified HPLC chromatograms of ALM-488 solutions at different solution pH values that are stored at 40° C. for 24 hours;

FIG. 10 is an overlay of magnified chromatograms of ALM-488 solution (50 mg/mL) in water analyzed initially and after 7 days at different storage temperatures;

FIG. 11 is a pH titration curve showing pH as a function of added tromethamine (Tris) and how solubility of an HCl salt of ALM-488 is affected by pH;

FIG. 12 is a pH titration curve showing pH as a function of added meglumine (N-methyl glucamine) and how solubility of an HCl salt of ALM-488 is affected by pH;

FIG. 13 is a pH titration curve showing pH as a function of added L-arginine and how solubility of an HCl salt of ALM-488 is affected by pH;

FIG. 14 is an overlay of magnified HPLC chromatograms for the Sample 8-4 of Example 8 at initial preparation, after storage at 40° C. for 7 days, and after storage at 40° C. for 14 days;

FIG. 15 is an overlay of magnified HPLC chromatographs for Samples 8-1 thru 8-7 of Example 8 after storage at 40° C. for 14 days;

FIG. 16 is an overlay of DSC heating curves for the arginine-containing ALM-488 formulations of Samples 8-1, 8-3, 8-4 and 8-7;

FIG. 17 is an overlay of DSC heating curves for the meglumine-containing ALM-488 formulations of Samples 8-2;

FIG. 18 shows a freeze-drying process graph for Sample 10-1;

FIG. 19 shows a freeze-drying process graph for Sample 10-2;

FIG. 20 shows a freeze-drying process graph for Sample 10-3;

FIG. 21 shows side view of lyophilized cakes of Samples 10-1 and 10-2;

FIG. 22 shows a top-down view of the dislodged lyphilized cake of Sample 10-2;

FIG. 23 shows the PXRD diffraction pattern for the lyophilized Samples 10-1;

FIG. 24 shows the PXRD diffraction pattern for the lyophilized Samples 10-2;

FIG. 25 shows the PXRD diffraction pattern for the lyophilized Samples 10-3;

FIG. 26 shows a freeze-drying process graph for Sample 11-1;

FIG. 27 is an overlay of HPLC chromatographs for the ALM-488/arginine-trehalose lyophile after storage are 40° C. at 75% relative humidity for periods of 1, 2 and 3 months;

FIG. 28 is an overlay of magnified HPLC chromatographs for the ALM-488/arginine-trehalose lyophile after storage are 40° C. at 75% relative humidity for periods of 1, 2 and 3 months;

FIG. 29 shows the PXRD diffraction pattern for the lyophilized Sample 10-3 after being stored at ° C. and 75% relative humidity for a period of three months.

### DETAILED DESCRIPTION

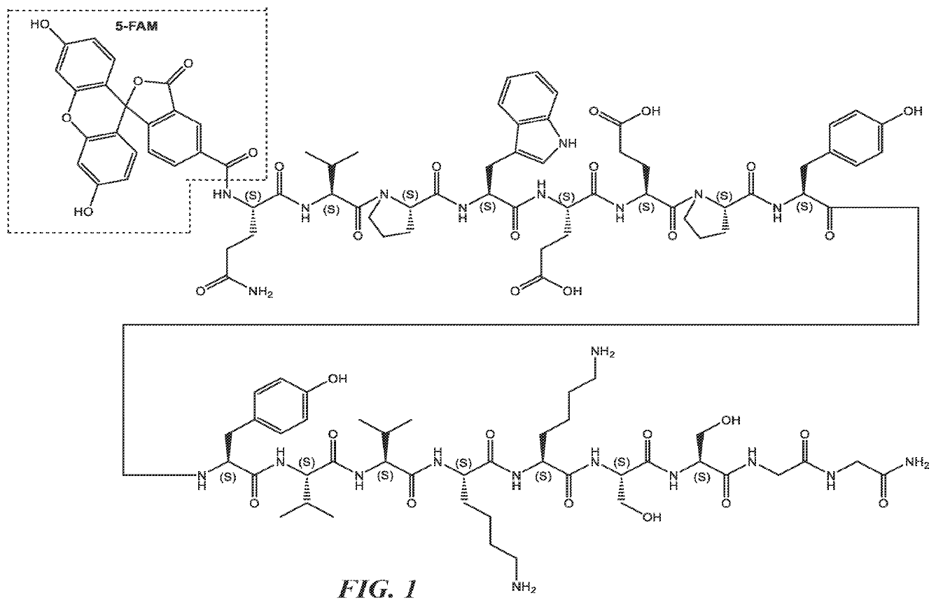

The present disclosure relates to liquid and solid compositions containing the nerve targeting peptide Bevonescein (ALM-488) (see FIG. 1) that are suitable for administration (e.g., intravenous administration) to subjects even after long-term storage of the compositions at elevated temperature and/or humidity. Although formulation of ALM-488 has proved to be challenging, compositions and related methods of the present disclosure enable this nerve targeting peptide to be safely stored under less-than-ideal conditions for lengthy periods of time without significant decomposition, followed by intravenous administration at predicable and reproducible doses without the need for additional purification or optimization. Examples 8-13 of the present disclosure illustrates these advantages.

### Terms and Definitions

Prior to setting forth this disclosure in more detail, it may be helpful to an understanding thereof to provide definitions of certain terms to be used herein. Additional definitions are set forth throughout this disclosure.

The term “consisting essentially of” limits the scope of a claim to the specified materials or steps and those that do not materially affect the basic and novel characteristics of the claimed invention. It should be understood that the terms “a” and “an” as used herein refer to “one or more” of the enumerated components.

The use of the alternative (e.g., “or”) should be understood to mean either one, both, or any combination thereof of the alternatives.

As used herein, the terms “include” and “have” are used synonymously, which terms and variants thereof are intended to be construed as non-limiting. The term “comprise” means the presence of the stated features, integers, steps, or components as referred to in the claims, but that it does not preclude the presence or addition of one or more other features, integers, steps, components, or groups thereof.

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, are to be understood to include any integer within the recited range, unless otherwise indicated.

As used herein, ranges and amounts can be expressed as “about” a particular value or range. About also includes the exact amount. Hence, for example, a pH value of “about 7” means a pH of about 7 and also means a pH of 7. In some embodiments, “about” means within 5% of the value. Hence, a pH of “about 7” means a pH ranging from 6.65 to 7.35. In some embodiments, “about” means within 4% of the value. In some embodiments, “about” means within 3% of the value. In some embodiments, “about” means within 2% of the value. In some embodiments, “about” means within 1% of the value. Generally, the term “about” includes an amount that would be expected to be within experimental error.

As used herein, the term “ambient temperature” means room temperature, i.e., ~20° C. to ~25° C. (~68° F. to ~77° F.).

As used herein, the term “aqueous vehicle” refers to a water-containing liquid that is suitable for intravenous injection to a subject.

As used herein, the term “pH-adjusting agent” refers to a basic, acidic or amphoteric organic compound that is suitable for adjusting pH of an aqueous solution.

As used herein, the term “buffering agent” refers to a basic, acidic or amphoteric organic compound that, when contained in an aqueous solution, resists changes in pH of the aqueous solution.

As used herein, the term “bulking agent” refers to agents that can be added to a pharmaceutical product to provide structure, volume and/or mass to the pharmaceutical product, thereby facilitating more precise metering and handling thereof.

As used herein, the term “tonicity-adjusting agent” refers to a compound that modifies the osmolality of a formulation, for example, to render it isotonic.

As used herein, the term “preservative” refers to a substance that prevents or inhibits microbial growth and extends the shelf life of an aqueous formulation.

As used herein, the term “elegant lyophilized cake” refers to a lyophilized composition (i.e., lyophile) that having the appearance of a uniform foamy plug with little shrinkage and minimal cracking.

As used herein, the term “homogeneous” refers to a liquid solution having only a single phase (i.e., with no solid phase and no liquid partition). In some embodiments, the term “homogenous” refers to an aqueous composition that contains less than 5% by weight of the nerve targeting peptide conjugate (i.e., Bevonescein (ALM-488)) in solid state based on filtration of the aqueous composition through a 0.22-micron Millipak Durapore® capsule filter unit.

As used herein, the terms “lyophilization,” “lyophilized” and “freeze-dried” refer to a process by which the material to be dried is first frozen and then the ice or frozen solvent is removed by sublimation in a vacuum environment.

As used herein, the term “lyophile” refers to a freeze dried solid.

As used herein, the term “reconstituted composition” refers to an aqueous composition formed by dissolving a lyophile in an aqueous vehicle.

As used herein, the terms “salt” or “salts” refers to an acid addition or base addition salt of a compound described herein. “Salts” include in particular “pharmaceutical acceptable salts.”

The term “pharmaceutically acceptable” as used herein, refers to a material that does not abrogate the biological activity or properties of the agents described herein, and is relatively nontoxic (i.e., the toxicity of the material significantly outweighs the benefit of the material). In some instances, a pharmaceutically acceptable material may be administered to an individual without causing significant undesirable biological effects or significantly interacting in a deleterious manner with any of the components of the composition in which it is contained.

As used herein, “pharmaceutically acceptable excipient” or “pharmaceutically acceptable carrier” as used herein means a pharmaceutically acceptable material, composition or vehicle, such as a liquid or solid filler, diluent, excipient, solvent or encapsulating material, and having the properties of being nontoxic and non-inflammatory in a patient. Each excipient must be “acceptable” in the sense of being compatible with the other ingredients of the formulation and not injurious to the patient. Excipients may include, for example: anti-adherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspending or dispersing agents, sweeteners, or waters of hydration. Examples of pharmaceutical excipients can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as excipients, particularly for injectable solutions. Suitable pharmaceutical excipients are described in “Remington's Pharmaceutical Sciences” by E. W. Martin.

As used herein, the “central nervous system” or “CNS” refers to the brain and the spinal cord, as well as the retina.

As used herein, the “peripheral nervous system” or “PNS” refers to the nerves and ganglia outside the CNS. The PNS is divided into the somatic nervous system and the autonomic nervous system. The somatic nervous system controls all voluntary muscular systems within the body, and the process of voluntary reflex arcs and is composed of afferent or sensory nerves, which relay sensation from the body to the central nervous system, and efferent or motor nerves, which send out commands from the CNS to the body, stimulating muscle contraction. The autonomic nervous system supplies smooth muscle and glands, and thus influences the function of internal organs. The autonomic nervous system acts largely unconsciously and regulates bodily functions, such as the heart rate, digestion, respiratory rate, pupillary response, urination, and sexual arousal.

As used herein, a “neuron” refers to an electrically excitable cell that processes and transmits information by electrical and chemical signaling. A typical neuron possesses a cell body (often called the soma), dendrites, and an axon.

As used herein, a “nerve” refers to an enclosed, cable-like bundle of neural axons. Each nerve is a cordlike structure that contains many axons. Each axon within the nerve is an extension of an individual neuron. Within a nerve, each axon is surrounded by a layer of connective tissue called the endoneurium. The axons are bundled together into groups called fascicles, and each fascicle is wrapped in a layer of connective tissue called the perineurium. Each nerve is covered on the outside by a dense sheath of connective tissue, the epineurium.

As used herein the term “conjugate” refers to a peptide attached to at least one cargo molecule, optionally via a linker.

The terms “individual,” “patient,” or “subject” are used interchangeably. As used herein, they mean any mammal (i.e., species of any orders, families, and genus within the taxonomic classification animalia: chordata: vertebrata: mammalia). In some embodiments, the mammal is a cow, horse, sheep, pig, cat, dog, goat, mouse, rat, rabbit, guinea pig, non-human primate, or human.

The terms “administer,” “administering”, “administration,” and the like, as used herein, refer to the methods that may be used to enable delivery of agents or compositions to the desired site of biological action. These methods include, but are not limited to topical, oral, intrarectal, intravaginal, intranasal, inhalation, parenteral injection (e.g., intravenous, intraarterial, subcutaneous, intraperitoneal, intramuscular, intravascular, intrathecal, intravitreal, infusion, or local). Administration techniques that are optionally employed with the agents and methods described herein, include e.g., as discussed in Goodman and Gilman, The Pharmacological Basis of Therapeutics, current ed.; Pergamon; and Remington's, Pharmaceutical Sciences (current edition), Mack Publishing Co., Easton, Pa. Administration of agents may be local or systemic. In some embodiments, administration is via systemic intravenous injection into human patients.

The term “surgery” as used herein, refers to any methods for that may be used to manipulate, change, or cause an effect by a physical intervention. These methods include, but are not limited to open surgery, endoscopic surgery, laparoscopic surgery, minimally invasive surgery, robotic surgery, any procedures that may affect any neuron or nerves such as placement of retractors during spinal surgery, microscopic procedures, cardiac neuron or nerve ablation, epidural injection, intrathecal injections, neuron or nerve blocks, implantation of devices such as neuron or nerve stimulators and implantation of pumps. In some embodiments, the subject of the surgery is a human subject or human patient.

### Pharmaceutical Compositions and Preparation Methods

Disclosed herein, in certain embodiments, are pharmaceutical compositions comprising the nerve targeting peptide conjugate “Bevonescein (ALM-488)” (i.e., SEQ ID NO:104 of WO2019/028281), which is shown below as formula (I), as well as pharmaceutically acceptable salts, solvates, hydrates, isomers, tautomers, racemates, or isotopes thereof.

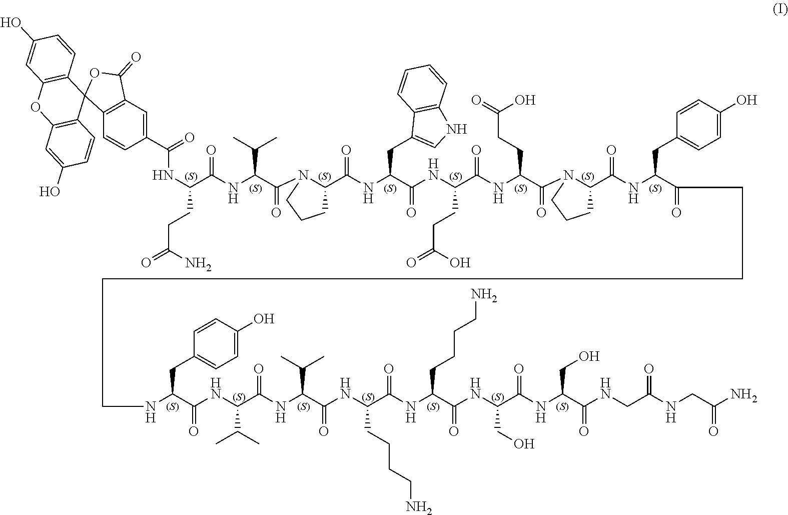

In some embodiments, the purity of the ALM-488 used to prepare the aqueous and solid (e.g., lyophilized) compositions described below is at least 95% by mass based on a total mass of the ALM-488. In other embodiments the purity of the ALM-488 is at least 96.0% by mass, or at least 96.5% by mass, or at least 97.0% by mass, or at least 97.5% by mass, or at least 98.0% by mass, or at least 98.5% by mass, or at least 99.0% by mass, or at least 99.5% by mass, based on the total mass of the ALM-488.

In some embodiments, the ALM-488 peptide used to prepare the aqueous and solid (e.g., lyophilized) compositions described below is in the form of a pharmaceutically-acceptable salt. For example, the pharmaceutically-acceptable salt can be formed from an inorganic acid, an organic acid, or a combination thereof. In some embodiments, the pharmaceutically-acceptable salt is an acetate salt, an aspartate salt, a benzoate salt, a besylate salt, a bromide salt, a bicarbonate salt, a carbonate salt, a chloride salt, a bisulfate salt, a sulfate salt, a camphorsulfonate salt, a chloride salt, a chlorotheophyllonate salt, a citrate salt, an ethandisulfonate salt, a fumarate salt, a gluceptate salt, a gluconate salt, a glucuronate salt, a hippurate salt, an iodide salt, an isethionate salt, a lactate salt, a lactobionate salt, a laurylsulfate salt, a malate salt, a maleate salt, a malonate salt, a mandelate salt, a mesylate salt, a methylsulphate salt, a naphthoate salt, a napsylate salt, a nicotinate salt, a nitrate salt, an octadecanoate salt, an oleate salt, an oxalate salt, a palmitate salt, a pamoate salt, a phosphate salt, a hydrogen phosphate salt, a dihydrogen phosphate salt, a polygalacturonate salt, a propionate salt, a stearate salt, a succinate salt, a sulfate salt, a sulfosalicylate salt, a tartrate salt, a tosylate, salt, a trifluoroacetate salt, and the like, or any combination thereof.

Pharmaceutical compositions herein are formulated using one or more physiologically acceptable carriers including excipients and auxiliaries which facilitate processing of the compound of formula (I) into preparations which are used pharmaceutically. Proper formulation is dependent upon the route of administration chosen. A summary of pharmaceutical compositions can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N. Y., 1980; and Pharmaceutical Dosage Forms and Drug Delivery Systems, Seventh Ed. (Lippincott Williams & Wilkins, 1999), the entire contents of each of which are incorporated herein by reference.

Compositions of the present disclosure (i.e., the aqueous pharmaceutical compositions, lyophilized pharmaceutical compositions, and reconstituted aqueous pharmaceutical compositions described below) may contain at least one pharmaceutically acceptable excipient (also referred to herein as an “additive”). Such additives may include, for example, pH-adjusting agents, buffering agents, bulking agents, tonicity-adjusting agents, preservatives, solvents, and combinations thereof. See, e.g., Pramanick et al., Excipient selection in parenteral formulation development, Pharma Times, 2013, 45 (3), 65-77, the entire contents of which are incorporated herein by reference.

pH-adjusting agents that may be contained in compositions of the present disclosure include, for example, alanine, arginine, a cellulose, cellobiose, a cyclodextrin, dextrose, polydextrose, erythritol, fructose, galactose, glucose, glycerin, glycine, histidine, inositol, lactose, maltose, mannitol, mannose, N-methyl glucamine (meglumine), raffinose, ribose, sorbitol, sucrose, trehalose, tromethamine, xylitol, xylose, and the like, or any combination thereof.

Buffering agents that may be contained in compositions of the present disclosure include, for example, calcium acetate, sodium acetate, potassium bitartrate, sodium borate, calcium chloride, potassium chloride, sodium chloride, citric acid, potassium citrate, sodium citrate, lactic acid, potassium lactate, sodium lactate, ammonium phosphates (mono- and di-basic), potassium phosphates (mono- and di-basic), sodium phosphates (mono-basic and di-basic), succinic acid, magnesium sulfate, or any combination thereof.

Buffers that may be contained in compositions of the present disclosure include, for example, a Tris buffer, a Tris-Cl buffer, a histidine buffer, a TAE buffer, a HEPES buffer, a TBE buffer, a sodium phosphate buffer, an MES buffer, an ammonium sulfate buffer, a potassium phosphate buffer, a potassium thiocyanate buffer, a succinate buffer, a tartrate buffer, a DIPSO buffer, a HEPPSO buffer, a POPSO buffer, a PIPES buffer, a PBS buffer, a MOPS buffer, an acetate buffer, a phosphate buffer, a cacodylate buffer, a glycine buffer, a sulfate buffer, an imidazole buffer, a guanidine hydrochloride buffer, a phosphate-citrate buffer, a borate buffer, a malonate buffer, a 3-picoline buffer, a 2-picolie buffer, a 4-picoline buffer, a 3,5-lutidine buffer, a 3,4-lutidine buffer, a 2,4-litidine buffer, a Aces buffer, a diethylmalonate buffer, an N-methylimidazole buffer, a 1,2-dimethylimidazole buffer, a TAPS buffer, a bis-Tris buffer, an L-arginine buffer, a lactate buffer, a glycolate buffer, and the like, or any combination thereof.

Bulking agents that may be contained in compositions of the present disclosure include, for example, alanine, arginine, calcium chloride, calcium sulfate, a cellulose, dextran 40, a dextrin (e.g., maltodextrin), dextrose, a polydextrose, dicalcium phosphate dihydrate, ethylene glycol, diethylene glycol, triethylene glycol, a polyethyleneglycol (PEG), a β-glucan, glycerin, diglycerin, a polyglycerin, glycine, histidine, a hydrocolloid (e.g., gum Arabic, pectin, guar gum, alginate, carrageenan, xanthan gum, cellulose gum, etc.), inulin, kaolin, magnesium chloride, magnesium aluminum silicate, meglumine (N-methyl glucamine), a monosaccharide (e.g., glucose, galactose, mannose, fructose, inositol, ribose, xylose, etc.), a disaccharide (e.g., lactose, sucrose, cellobiose, trehalose, maltose, etc.), a trisaccharide (e.g., raffinose, etc.), a polysaccharide (e.g., cyclodextrin, etc.), a sugar alcohol (e.g., erythritol, xylitol, sorbitol, mannitol, maltitol, etc.) potassium chloride, propylene glycol (PG), polypropylene glycol, a polyvinylpyrrolidone (PVP), a poloxamer, silicon dioxide, sodium chloride, a starch, titanium oxide, tromethamine, or any combination thereof.

Tonicity-adjusting agents that may be contained in compositions of the present disclosure include, for example, alanine, arginine, calcium chloride, calcium sulfate, a cellulose, dextran 40, a dextrin (e.g., maltodextrin), dextrose, a polydextrose, dicalcium phosphate dihydrate, ethylene glycol, diethylene glycol, triethylene glycol, a polyethyleneglycol (PEG), a β-glucan, glycerin, diglycerin, a polyglycerin, glycine, histidine, a hydrocolloid (e.g., gum Arabic, pectin, guar gum, alginate, carrageenan, xanthan gum, cellulose gum, etc.), inulin, kaolin, magnesium chloride, magnesium aluminum silicate, meglumine (N-methyl glucamine), a monosaccharide (e.g., glucose, galactose, mannose, fructose, inositol, ribose, xylose, etc.), a disaccharide (e.g., lactose, sucrose, cellobiose, trehalose, maltose, etc.), a trisaccharide (e.g., raffinose, etc.), a polysaccharide (e.g., cyclodextrin, etc.), a sugar alcohol (e.g., erythritol, xylitol, sorbitol, mannitol, maltitol, etc.) potassium chloride, propylene glycol (PG), polypropylene glycol, a polyvinylpyrrolidone (PVP), a poloxamer, silicon dioxide, sodium chloride, a starch, titanium oxide, tromethamine, or any combination thereof.

Preservatives that may be contained in compositions of the present disclosure include, for example, benzoic acid, sodium benzoate, benzyl alcohol, benzalkonium chloride, bronidol, bronopol, butyl paraben, chlorobutanol, methyl cresol, chlorocresol, methyl paraben, phenol, phenoxyethanol, phenyl ethyl alcohol, propylene glycol, propyl paraben, sorbic acid, thimerosal, and the like, or any combination thereof.

In some embodiments, the pharmaceutical compositions of the present disclosure (i.e., the aqueous pharmaceutical compositions, lyophilized pharmaceutical compositions, and reconstituted aqueous pharmaceutical compositions described below) may include other medicinal or pharmaceutical agents, carriers, adjuvants, such as preserving, stabilizing, wetting or emulsifying agents, solution promoters, salts for regulating the osmotic pressure, and/or buffers. In addition, the pharmaceutical compositions also contain other therapeutically valuable substances.

### Aqueous Pharmaceutical Compositions

One embodiment relates to aqueous pharmaceutical compositions comprising (a) the fluorescein-labeled nerve targeting peptide of formula (I), or pharmaceutically acceptable salt, solvate, hydrate, isomer, tautomer, racemate, or isotope thereof; (b) an additive comprising a pH-adjusting agent, a buffering agent, a bulking agent, a tonicity-adjusting agent, a preservative, or any combination thereof, as described above; and (c) an aqueous vehicle. In some embodiments, a pH of the aqueous composition ranges from about 5 to about 12, and a concentration of the nerve targeting peptide in the aqueous composition ranges from about 10 mg/mL to about 500 mg/mL based on a total volume of the aqueous composition.

In some embodiments, the peptide of formula (I) (i.e., Bevonescein (ALM-488)) is in the form of a pharmaceutically acceptable salt as described above. For example, in some embodiments the aqueous pharmaceutical composition comprises an HCl salt of the nerve targeting peptide. A chloride content of the HCl salt may range from about 0.1% (w/w) to about 5.0% (w/w), or from about 0.1% (w/w) to about 1.0% (w/w), or from about 1.0% (w/w) to about 2.0% (w/w), or from about 2.0% (w/w) to about 3.0% (w/w), or from about 3.0% (w/w) to about 4.0% (w/w), or from about 4.0% (w/w) to about 5.0% (w/w), based on a total mass of the peptide in the aqueous composition.

In some embodiments, a molar ratio of the peptide to the pharmaceutically-acceptable salt in the aqueous composition ranges from about 1:1 to about 1:3, or from about 1:1 to about 1:2, or from about 1:1 to about 1:1.9, or from about 1:1 to about 1:1.8, or from about 1:1 to about 1.7, or from about 1:1 to about 1:1.6, or from about 1:1 to about 1:1.5, or from about 1:1 to about 1:1.4, or from about 1:1 to about 1:1.3, or from about 1:1 to about 1:1.2.

In some embodiments, the additive in the aqueous composition includes a pH-adjusting agent, a buffering agent, a buffer, a bulking agent, a tonicity-adjusting agent, a preservative, at least one solvent, and combinations thereof. Such additives may include, for example, the pH-adjusting agents, buffering agents, buffers, bulking agents, tonicity-adjusting agents, and preservatives as described above.

In some embodiments, the additive in the aqueous composition comprises alanine, arginine, a cellulose, cellobiose, a cyclodextrin, dextrose, erythritol, fructose, galactose, glucose, glycerin, glycine, histidine, inositol, lactose, maltose, mannitol, mannose, meglumine, raffinose, ribose, sorbitol, sucrose, trehalose, tromethamine, xylitol, xylose, or any combination thereof. In certain embodiments, the additive comprises arginine, glycine, mannitol, meglumine, trehalose, tromethamine, or any combination thereof.

In some embodiments, the additive in the aqueous composition includes a first additive comprising arginine, meglumine, glycine, or any combination thereof; and optionally a second additive comprising mannitol, trehalose, or both. In certain embodiments, a mass ratio of the first additive to the second additive ranges from about 10:1 to about 1:10, or from about 10:1 to about 9:1, or from about 9:1 to about 8:1, or from about 8:1 to about 7:1, or from about 7:1 to about 6:1, or from about 6:1 to about 5:1, or from about 5:1 to about 4:1, or from about 4:1 to about 3:1, or from about 3:1 to about 2:1, or from about 2:1 to about 1:0, or from about 1.8:1 to about 1.7:1, or from about 1.7:1 to about 1.6:1, or from about 1.6:1 to about 1.5:1, or from about 1.5:1 to about 1.4:1, or from about 1.4:1 to about 1.3:1, or from about 1.3:1 to about 1.2:1, or from about 1.2:1 to about 1.1:1, or from about 1:1.1 to about 1:1.2, or from about 1:1 to about 1:2.5, or from about 1:1.2 to about 1:1.3, or from about 1:1.3 to about 1:1.4, or from about 1:1.4 to about 1:1.5, or from about 1:1.5 to about 1:1.6, or from about 1:1.6 to about 1:1.8, or from about 1:1.8 to about 1:1.9, or from about 1:1.9 to about 1:2, or from about 1:2 to about 1:3, or from about 1:3 to about 1:4, or from about 1:4 to about 1:5, or from about 1:5 to about 1:6, or from about 1:6 to about 1:7, or from about 1:7 to about 1:8, or from about 1:8 to about 1:9, or from about 1:9 to about 1:10.

In some embodiments, the additive in the aqueous composition comprises arginine, arginine-mannitol, arginine-trehalose, arginine-meglumine, meglumine, meglumine-glycine, or meglumine-mannitol. In certain embodiments, the additive comprises arginine-trehalose. In certain embodiments, a mass ratio of arginine to trehalose ranges from about 10:1 to about 1:10, or from about 10:1 to about 9:1, or from about 9:1 to about 8:1, or from about 8:1 to about 7:1, or from about 7:1 to about 6:1, or from about 6:1 to about 5:1, or from about 5:1 to about 4:1, or from about 4:1 to about 3:1, or from about 3:1 to about 2:1, or from about 2:1 to about 1:0, or from about 1.8:1 to about 1.7:1, or from about 1.7:1 to about 1.6:1, or from about 1.6:1 to about 1.5:1, or from about 1.5:1 to about 1.4:1, or from about 1.4:1 to about 1.3:1, or from about 1.3:1 to about 1.2:1, or from about 1.2:1 to about 1.1:1, or from about 1:1.1 to about 1:1.2, or from about 1:1 to about 1:2.5, or from about 1:1.2 to about 1:1.3, or from about 1:1.3 to about 1:1.4, or from about 1:1.4 to about 1:1.5, or from about 1:1.5 to about 1:1.6, or from about 1:1.6 to about 1:1.8, or from about 1:1.8 to about 1:1.9, or from about 1:1.9 to about 1:2, or from about 1:2 to about 1:3, or from about 1:3 to about 1:4, or from about 1:4 to about 1:5, or from about 1:5 to about 1:6, or from about 1:6 to about 1:7, or from about 1:7 to about 1:8, or from about 1:8 to about 1:9, or from about 1:9 to about 1:10.

In some embodiments, a mass ratio of the peptide to the additive in the aqueous composition ranges from about 5:1 to about 1:5. In certain embodiments, the mass ratio of the peptide to the additive in the aqueous composition ranges from about 5:1 to about 4.5:1, or from about 4.5:1 to about 4.0:1, or from about 4.0:1 to about 3.5:1, or from about 3.5:1 to about 3.0:1, or from about 3.0:1 to about 2.5:1, or from about 2.5:1 to about 2.0:1, or from about 2.0:1 to about 1.5:1, or from about 1.5:1 to about 1.0:1, or from about 1.0:1 to about 0.5:1, or from about 1:0.5 to about 1:1.0, or from about 1:1.0 to about 1:1.5, or from about 1:1.5 to about 1:2.0, or from about 1:2.0 to about 1:2.5, or from about 1:2.5 to about 1:3.0, or from about 1:3.0 to about 1:3.5, or from about 1:3.5 to about 1:4.0, or from about 1:4.0 to about 1:4.5, or from about 1:4.5 to about 1:4.0. In other embodiments, the mass ratio of the peptide to the additive ranges from about 1.0 to about 1.5.

In some embodiments, the aqueous vehicle in the aqueous composition comprises a sterile water, a saline solution, an aqueous buffer, or any combination thereof. In certain embodiments, the aqueous vehicle comprises a sterile water, a normal saline, a half-normal saline, or any combination thereof.

In some embodiments, the pH of the aqueous composition ranges from about 5 to about 12, or from about 5 to about 6, or from about 6 to about 7, or from about 7.0 to about 7.1, or from about 7.1 to about 7.2, or from about 7.2 to about 7.3, or from about 7.3 to about 7.4, or from about 7.4 to about 7.5, or from about 7.5 to about 7.6, or from about 7.6 to about 7.7, or from about 7.7 to about 7.8, or from about 7.8 to about 7.9, or from about 7.9 to about 8.0, or from about 8.0 to about 8.1, or from about 8.1 to about 8.2, or from about 8.2 to about 8.3, or from about 8.3 to about 8.4, or from about 8.4 to about 8.5, or from about 8.5 to about 8.6, or from about 8.6 to about 8.7, or from about 8.7, to about 8.8, or from about 8.8 to about 8.9, or from about 8.9 to about 9.0, or from about 9 to about 10, or from about 10 to about 11, of from about 11 to about 12. In certain embodiments, the pH of the aqueous composition ranges from about 7.2 to about 7.8.

In some embodiments, a concentration of the peptide (i.e., Bevonescein (ALM-488)) in the aqueous composition ranges from about 20 mg/mL to about 30 mg/mL, or from about 30 mg/mL to about 40 mg/mL, or from about 40 mg/mL to about 50 mg/mL, or from about 50 mg/mL to about 60 mg/mL, or from about 60 mg/mL to about 70 mg/mL, or from about 70 mg/mL to about 80 mg/mL, or from about 80 mg/mL to about 90 mg/mL, or from about 90 mg/mL to about 100 mg/mL, or from about 100 mg/mL to about 110 mg/mL, or from about 110 mg/mL to about 120 mg/mL, or from about 120 mg/mL to about 130 mg/mL, or from about 130 mg/mL to about 140 mg/mL, or from about 140 mg/mL to about 150 mg/mL, or from about 150 mg/mL to about 160 mg/mL, or from about 160 mg/mL to about 170 mg/mL, or from about 170 mg/mL to about 180 mg/mL, or from about 180 mg/mL to about 190 mg/mL or from about 190 mg/mL to about 200 mg/mL, or from about 200 mg/mL to about 250 mg/mL, or from about 250 mg/mL to about 300 mg/mL, or from about 300 mg/mL to about 350 mg/mL, or from about 350 mg/mL to about 400 mg/mL, or from about 400 mg/mL to about 450 mg/mL, based on a total volume of the aqueous composition. In certain embodiments, a concentration of the peptide in the aqueous composition ranges from about 25 mg/mL to about 200 mg/mL based on the total volume of the aqueous composition. In other embodiments, a concentration of the peptide in the aqueous composition ranges from about 40 mg/mL to about 75 mg/mL based on the total volume of the aqueous composition.

In some embodiments, a density of the aqueous composition ranges from about 0.6 g/mL to about 1.3 g/mL. In certain embodiments the density of the aqueous compositions ranges from about 0.6 g/mL to about 0.7 g/mL, or from about 0.7 g/mL to about 0.8 g/mL, or from about 0.8 g/mL to about 0.85 g/mL, or from about 0.85 g/mL to about 0.90 g/mL, or from about 0.90 g/mL to about 0.95 g/mL, or from about 0.95 g/mL to about 1.0 g/mL, or from about 1.0 g/mL to about 1.05 g/mL, or from about 1.05 g/mL to about 1.10 g/mL, or from about 1.10 g/mL to about 1.15 g/mL, or from about 1.15 g/mL to about 1.20 g/mL, or from about 1.20 g/mL to about 1.25 g/mL. In other embodiments, the density of the aqueous composition ranges from about 1.0 g/mL to about 1.08 g/mL.

In some embodiments, an osmolality of the aqueous composition ranges from about 150 mOsm/kg to about 400 mOsm/kg. In certain embodiments the osmolality of the aqueous composition ranges from about 150 mOsm/kg to about 175 mOsm/kg, or from about 175 mOsm/kg to about 200 mOsm/kg, or from about 200 mOsm/kg to about 225 mOsm/kg, or from about 225 mOsm/kg to about 250 mOsm/kg, or from about 250 mOsm/kg to about 275 mOsm/kg, or from about 275 mOsm/kg to about 300 mOsm/kg, or from about 300 mOsm/kg to about 325 mOsm/kg, or from about 325 mOsm/kg to about 350 mOsm/kg. In other embodiments the osmolality of the aqueous composition ranges from about 280 mOsm/kg to about 300 mOsm/kg.

In some embodiments, a volume of the aqueous composition ranges from about 0.5 mL to about 5.0 mL. In certain embodiments, the volume the aqueous composition ranges from about 0.5 mL to about 1.0 mL, or from about 1.0 mL to about 1.5 mL, or from about 1.5 mL to about 2.0 mL, or from about 2.0 mL to about 2.5 mL, or from about 2.5 mL to about 3.0 mL, or from about 3.0 mL to about 3.5 mL, or from about 3.5 mL to about 4.0 mL, or from about 4.0 mL to about 4.5 mL. In other embodiments the volume of the aqueous composition ranges from about 1.8 mL to about 2.2 mL.

In some embodiments, the aqueous composition is filtered. Whereas in other embodiments the aqueous composition is non-filtered. In certain embodiments, the aqueous composition is homogeneous.

In some embodiments, the aqueous composition comprises at least 95 mass % of the peptide (i.e., Bevonescein (ALM-488)), as measured relative to a total mass of the aqueous composition, after storage at 40° C. for a period of 14 days. In certain embodiments, the aqueous composition comprises at least 96.0 mass %, or at least 96.5 mass %, or at least 97.0 mass %, or at least 97.5 mass %, or at least 98.0 mass %, or at least 98.5 mass %, or at least 99.0 mass %, or at least 99.5 mass %, of the peptide, as measured relative to a total mass of the aqueous composition, after storage at 40° C. for a period of 14 days.

### Lyophilized Pharmaceutical Compositions and Preparation Methods

Another embodiment relates to a lyophilized pharmaceutical composition comprising (a) the fluorescein-labeled nerve targeting peptide of formula (I) (i.e., Bevonescein (ALM-488)), or pharmaceutically acceptable salt, solvate, hydrate, isomer, tautomer, racemate, or isotope thereof; and (b) an additive comprising a pH-adjusting agent, a buffering agent, a bulking agent, a tonicity-adjusting agent, a preservative, or any combination thereof, wherein a water content of the lyophilized composition ranges from about 0.2% (w/w) to about 2.0% (w/w), relative to a total mass of the lyophilized composition.

In some embodiments, the lyophilized composition forms a homogeneous aqueous composition having a peptide (i.e., Bevonescein (ALM-488)) concentration ranging from about 50 mg/mL to about 500 mg/mL when combined with an aqueous vehicle at ambient temperature. In certain embodiments, the peptide concentration of a homogeneous aqueous composition formed from the lyophilized composition ranges from about 50 mg/mL to about 75 mg/mL, or from about 75 mg/mL to about 100 mg/mL, or from about 100 mg/mL to about 125 mg/mL, or from about 125 mg/mL to about 150 mg/mL, or from about 150 mg/mL to about 175 mg/mL, or from about 175 mg/mL to about 200 mg/mL, or from about 200 mg/mL to about 225 mg/mL, or from about 225 mg/mL to about 250 mg/mL, or from about 250 mg/mL to about 275 mg/mL, or from about 275 mg/mL to about 300 mg/mL, or from about 300 mg/mL to about 325 mg/mL, or from about 325 mg/mL to about 350 mg/mL, or from about 350 mg/mL to about 375 mg/mL, or from about 375 mg/mL to about 400 mg/mL, or from about 400 mg/mL to about 425 mg/mL, or from about 425 mg/mL to about 450 mg/mL. In other embodiments the lyophilized composition forms a homogeneous aqueous composition having a peptide concentration of 200 mg/mL or less when combined with an aqueous vehicle at ambient temperature.

In some embodiments, the lyophilized composition is in the form of an elegant lyophilized cake.

In some embodiments, the lyophilized composition comprises at least 95 mass % of the peptide (i.e., Bevonescein (ALM-488)) after storage of the lyophilized composition at 40° C. and 75% relative humidity for a period of 3 months. In certain embodiments, the lyophilized composition comprises at least 95 mass %, or about least 95.5 mass %, or at least 96.0 mass %, or at least 96.5 mass %, or at least 97.0 mass %, or about 97.5 mass %, or at least 98.0 mass %, or at least 98.5 mass %, or about least 99.0 mass %, or at least 99.5 mass %, of the peptide (i.e., Bevonescein (ALM-488)) after storage of the lyophilized composition at 40° C. and 75% relative humidity for a period of 3 months.

In some embodiments, the peptide of formula (I) (i.e., Bevonescein (ALM-488)) is in the form of a pharmaceutically acceptable salt as described above. For example, in some embodiments the lyophilized composition comprises an HCl salt of the peptide. A chloride content of the HCl salt may range from about 0.1% (w/w) to about 5.0% (w/w), or from about 0.1% (w/w) to about 1.0% (w/w), or from about 1.0% (w/w) to about 2.0% (w/w), or from about 2.0% (w/w) to about 3.0% (w/w), or from about 3.0% (w/w) to about 4.0% (w/w), or from about 4.0% (w/w) to about 5.0% (w/w), based on a total mass of the peptide in the lyophilized composition.

In some embodiments, a molar ratio of the peptide to the pharmaceutically-acceptable salt in the lyophilized composition ranges from about 1:1 to about 1:3, or from about 1:1 to about 1:2, or from about 1:1 to about 1:1.9, or from about 1:1 to about 1:1.8, or from about 1:1 to about 1.7, or from about 1:1 to about 1:1.6, or from about 1:1 to about 1:1.5, or from about 1:1 to about 1:1.4, or from about 1:1 to about 1:1.3, or from about 1:1 to about 1:1.2.

In some embodiments, the additive contained in the lyophilized composition includes a pH-adjusting agent, a buffering agent, a buffer, a bulking agent, a tonicity-adjusting agent, a preservative, and combinations thereof. Such additives may include, for example, the pH-adjusting agents, buffering agents, buffers, bulking agents, tonicity-adjusting agents and preservatives as described above.

In some embodiments, the additive contained in the lyophilized composition comprises alanine, arginine, a cellulose, cellobiose, a cyclodextrin, dextrose, erythritol, fructose, galactose, glucose, glycerin, glycine, histidine, inositol, lactose, maltose, mannitol, mannose, meglumine, raffinose, ribose, sorbitol, sucrose, trehalose, tromethamine, xylitol, xylose, or any combination thereof. In certain embodiments, the additive comprises arginine, glycine, mannitol, meglumine, trehalose, tromethamine, or any combination thereof.

In some embodiments, the additive contained in the lyophilized composition includes a first additive comprising arginine, meglumine, glycine, or any combination thereof; and optionally a second additive comprising mannitol, trehalose, or both. In certain embodiments, a mass ratio of the first additive to the second additive ranges from about 10:1 to about 1:10, or from about 10:1 to about 9:1, or from about 9:1 to about 8:1, or from about 8:1 to about 7:1, or from about 7:1 to about 6:1, or from about 6:1 to about 5:1, or from about 5:1 to about 4:1, or from about 4:1 to about 3:1, or from about 3:1 to about 2:1, or from about 2:1 to about 1:0, or from about 1.8:1 to about 1.7:1, or from about 1.7:1 to about 1.6:1, or from about 1.6:1 to about 1.5:1, or from about 1.5:1 to about 1.4:1, or from about 1.4:1 to about 1.3:1, or from about 1.3:1 to about 1.2:1, or from about 1.2:1 to about 1.1:1, or from about 1:1.1 to about 1:1.2, or from about 1:1 to about 1:2.5, or from about 1:1.2 to about 1:1.3, or from about 1:1.3 to about 1:1.4, or from about 1:1.4 to about 1:1.5, or from about 1:1.5 to about 1:1.6, or from about 1:1.6 to about 1:1.8, or from about 1:1.8 to about 1:1.9, or from about 1:1.9 to about 1:2, or from about 1:2 to about 1:3, or from about 1:3 to about 1:4, or from about 1:4 to about 1:5, or from about 1:5 to about 1:6, or from about 1:6 to about 1:7, or from about 1:7 to about 1:8, or from about 1:8 to about 1:9, or from about 1:9 to about 1:10.

In some embodiments, the additive contained in the lyophilized composition comprises arginine, arginine-mannitol, arginine-trehalose, arginine-meglumine, meglumine, meglumine-glycine, or meglumine-mannitol. In certain embodiments, the additive comprises arginine-trehalose. In certain embodiments, a mass ratio of arginine to trehalose ranges from about 10:1 to about 1:10, or from about 10:1 to about 9:1, or from about 9:1 to about 8:1, or from about 8:1 to about 7:1, or from about 7:1 to about 6:1, or from about 6:1 to about 5:1, or from about 5:1 to about 4:1, or from about 4:1 to about 3:1, or from about 3:1 to about 2:1, or from about 2:1 to about 1:0, or from about 1.8:1 to about 1.7:1, or from about 1.7:1 to about 1.6:1, or from about 1.6:1 to about 1.5:1, or from about 1.5:1 to about 1.4:1, or from about 1.4:1 to about 1.3:1, or from about 1.3:1 to about 1.2:1, or from about 1.2:1 to about 1.1:1, or from about 1:1.1 to about 1:1.2, or from about 1:1 to about 1:2.5, or from about 1:1.2 to about 1:1.3, or from about 1:1.3 to about 1:1.4, or from about 1:1.4 to about 1:1.5, or from about 1:1.5 to about 1:1.6, or from about 1:1.6 to about 1:1.8, or from about 1:1.8 to about 1:1.9, or from about 1:1.9 to about 1:2, or from about 1:2 to about 1:3, or from about 1:3 to about 1:4, or from about 1:4 to about 1:5, or from about 1:5 to about 1:6, or from about 1:6 to about 1:7, or from about 1:7 to about 1:8, or from about 1:8 to about 1:9, or from about 1:9 to about 1:10.

In some embodiments, a mass ratio of the peptide to the additive in the lyophilized composition ranges from about 5:1 to about 1:5. In certain embodiments, the mass ratio of the peptide to the additive in the lyophilized composition ranges from about 5:1 to about 4.5:1, or from about 4.5:1 to about 4.0:1, or from about 4.0:1 to about 3.5:1, or from about 3.5:1 to about 3.0:1, or from about 3.0:1 to about 2.5:1, or from about 2.5:1 to about 2.0:1, or from about 2.0:1 to about 1.5:1, or from about 1.5:1 to about 1.0:1, or from about 1.0:1 to about 0.5:1, or from about 1:0.5 to about 1:1.0, or from about 1:1.0 to about 1:1.5, or from about 1:1.5 to about 1:2.0, or from about 1:2.0 to about 1:2.5, or from about 1:2.5 to about 1:3.0, or from about 1:3.0 to about 1:3.5, or from about 1:3.5 to about 1:4.0, or from about 1:4.0 to about 1:4.5, or from about 1:4.5 to about 1:4.0. In other embodiments, the mass ratio of the peptide to the additive ranges from about 1.0 to about 1.5.

Another embodiment relates to methods of forming a lyophilized pharmaceutical composition by lyophilizing an aqueous composition disclosed herein. In some embodiments, the lyophilizing comprises freezing the aqueous composition at a temperature ranging from about −100° C. to about −45° C. to obtain a frozen solid composition. In certain embodiments, the frozen solid composition is formed by freezing the aqueous composition at a temperature ranging from about −100° C. to about −90° C., or from about −90° C. to about −80° C., or from about −80° C. to about −70° C., or from about −70° C. to about −60° C., or from about −60° C. to about −50° C. In other embodiments, the lyophilizing comprises freezing the aqueous composition at a temperature ranging from about −80° C. to about −50° C. to obtain the frozen solid composition.

In some embodiments, the lyophilizing further comprises annealing the frozen solid composition at temperature ranging from about −40° C. to about −20° C. In certain embodiments, the annealing occurs at a temperature ranging from about −40° C. to about −35° C., or from about −35° C. to about −30° C., or from about −30° C. to about −25° C., or from about −25° C. to about −20° C.

In some embodiments, the lyophilizing further comprises drying the frozen solid composition at a temperature ranging from about −20° C. to room temperature, at a pressure ranging from about 100 mTorr to about 300 mTorr, to obtain the lyophilized composition. In certain embodiments, the drying temperature ranges from about −20° C. to about −15° C., or from about −15° C. to about −10° C., or from about −10° C. to about −5° C., or from about −5° C. to about 0° C., or from about 0° C. to about 5° C., or from about 5° C. to about 10° C., or from about 10° C. to about 15° C., or from about 15° C. to about 20° C., or from about 20° C. to about 25° C., at a pressure ranges from about 100 mTorr to about 300 mTorr.

In some embodiments, the drying comprises: (i) a primary drying of the frozen solid composition at a temperature ranging from about −20° C. to about 0° C. at a primary pressure ranging from about 100 mTorr to about 300 mTorr; and (ii) a secondary drying of the frozen solid composition at a temperature ranging from about 0° C. to room temperature at a secondary pressure ranging from about 100 mTorr to about 300 mTorr.

Another embodiment relates to lyophilized pharmaceutical compositions that are formed by the lyophilization methods described herein.

### Reconstituted Aqueous Pharmaceutical Compositions and Preparation Methods

Another embodiment relates to reconstituted aqueous pharmaceutical compositions comprising ALM-488, as well as pharmaceutically acceptable salts, solvates, hydrates, isomers, tautomers, racemates, or isotopes thereof, which are formed by combining lyophilized compositions described herein with an aqueous vehicle.

In some embodiments, the aqueous vehicle used to prepare the reconstituted aqueous composition comprises a sterile water, a saline solution, an aqueous buffer, or any combination thereof. In certain embodiments, the aqueous vehicle comprises a sterile water, a normal saline, a half-normal saline, or any combination thereof.

In some embodiments, the reconstituted aqueous composition is filtered. Whereas in other embodiments the reconstituted aqueous composition is non-filtered. In certain embodiments, the reconstituted composition is homogeneous.

In some embodiments, the peptide of formula (I) (i.e., Bevonescein (ALM-488)) that is contained in the reconstituted aqueous composition is in the form of a pharmaceutically acceptable salt as described above. For example, in some embodiments the reconstituted aqueous pharmaceutical composition comprises an HCl salt of the nerve targeting peptide. A chloride content of the HCl salt may range from about 0.1% (w/w) to about 5.0% (w/w), or from about 0.1% (w/w) to about 1.0% (w/w), or from about 1.0% (w/w) to about 2.0% (w/w), or from about 2.0% (w/w) to about 3.0% (w/w), or from about 3.0% (w/w) to about 4.0% (w/w), or from about 4.0% (w/w) to about 5.0% (w/w), based on a total mass of the peptide in the reconstituted aqueous composition.

In some embodiments, a molar ratio of the peptide to the pharmaceutically-acceptable salt in the reconstituted aqueous composition ranges from about 1:1 to about 1:3, or from about 1:1 to about 1:2, or from about 1:1 to about 1:1.9, or from about 1:1 to about 1:1.8, or from about 1:1 to about 1.7, or from about 1:1 to about 1:1.6, or from about 1:1 to about 1:1.5, or from about 1:1 to about 1:1.4, or from about 1:1 to about 1:1.3, or from about 1:1 to about 1:1.2.

In some embodiments, the additive in the reconstituted aqueous composition includes a pH-adjusting agent, a buffering agent, a buffer, a bulking agent, a tonicity-adjusting agent, a preservative, at least one solvent, and combinations thereof. Such additives may include, for example, the pH-adjusting agents, buffering agents, buffers, bulking agents, tonicity-adjusting agents and preservatives as described above.

In some embodiments, the additive in the reconstituted aqueous composition comprises alanine, arginine, a cellulose, cellobiose, a cyclodextrin, dextrose, erythritol, fructose, galactose, glucose, glycerin, glycine, histidine, inositol, lactose, maltose, mannitol, mannose, meglumine, raffinose, ribose, sorbitol, sucrose, trehalose, tromethamine, xylitol, xylose, or any combination thereof. In certain embodiments, the additive comprises arginine, glycine, mannitol, meglumine, trehalose, tromethamine, or any combination thereof.

In some embodiments, the additive in the reconstituted aqueous composition includes a first additive comprising arginine, meglumine, glycine, or any combination thereof; and optionally a second additive comprising mannitol, trehalose, or both. In certain embodiments, a mass ratio of the first additive to the second additive ranges from about 10:1 to about 1:10, or from about 10:1 to about 9:1, or from about 9:1 to about 8:1, or from about 8:1 to about 7:1, or from about 7:1 to about 6:1, or from about 6:1 to about 5:1, or from about 5:1 to about 4:1, or from about 4:1 to about 3:1, or from about 3:1 to about 2:1, or from about 2:1 to about 1:0, or from about 1.8:1 to about 1.7:1, or from about 1.7:1 to about 1.6:1, or from about 1.6:1 to about 1.5:1, or from about 1.5:1 to about 1.4:1, or from about 1.4:1 to about 1.3:1, or from about 1.3:1 to about 1.2:1, or from about 1.2:1 to about 1.1:1, or from about 1:1.1 to about 1:1.2, or from about 1:1 to about 1:2.5, or from about 1:1.2 to about 1:1.3, or from about 1:1.3 to about 1:1.4, or from about 1:1.4 to about 1:1.5, or from about 1:1.5 to about 1:1.6, or from about 1:1.6 to about 1:1.8, or from about 1:1.8 to about 1:1.9, or from about 1:1.9 to about 1:2, or from about 1:2 to about 1:3, or from about 1:3 to about 1:4, or from about 1:4 to about 1:5, or from about 1:5 to about 1:6, or from about 1:6 to about 1:7, or from about 1:7 to about 1:8, or from about 1:8 to about 1:9, or from about 1:9 to about 1:10.

In some embodiments, the additive in the reconstituted aqueous composition comprises arginine, arginine-mannitol, arginine-trehalose, arginine-meglumine, meglumine, meglumine-glycine, or meglumine-mannitol. In certain embodiments, the additive comprises arginine-trehalose. In certain embodiments, a mass ratio of arginine to trehalose ranges from about 10:1 to about 1:10, or from about 10:1 to about 9:1, or from about 9:1 to about 8:1, or from about 8:1 to about 7:1, or from about 7:1 to about 6:1, or from about 6:1 to about 5:1, or from about 5:1 to about 4:1, or from about 4:1 to about 3:1, or from about 3:1 to about 2:1, or from about 2:1 to about 1:0, or from about 1.8:1 to about 1.7:1, or from about 1.7:1 to about 1.6:1, or from about 1.6:1 to about 1.5:1, or from about 1.5:1 to about 1.4:1, or from about 1.4:1 to about 1.3:1, or from about 1.3:1 to about 1.2:1, or from about 1.2:1 to about 1.1:1, or from about 1:1.1 to about 1:1.2, or from about 1:1 to about 1:2.5, or from about 1:1.2 to about 1:1.3, or from about 1:1.3 to about 1:1.4, or from about 1:1.4 to about 1:1.5, or from about 1:1.5 to about 1:1.6, or from about 1:1.6 to about 1:1.8, or from about 1:1.8 to about 1:1.9, or from about 1:1.9 to about 1:2, or from about 1:2 to about 1:3, or from about 1:3 to about 1:4, or from about 1:4 to about 1:5, or from about 1:5 to about 1:6, or from about 1:6 to about 1:7, or from about 1:7 to about 1:8, or from about 1:8 to about 1:9, or from about 1:9 to about 1:10.

In some embodiments, a mass ratio of the peptide to the additive in the reconstituted aqueous composition ranges from about 5:1 to about 1:5. In certain embodiments, the mass ratio of the peptide to the additive in the aqueous composition ranges from about 5:1 to about 4.5:1, or from about 4.5:1 to about 4.0:1, or from about 4.0:1 to about 3.5:1, or from about 3.5:1 to about 3.0:1, or from about 3.0:1 to about 2.5:1, or from about 2.5:1 to about 2.0:1, or from about 2.0:1 to about 1.5:1, or from about 1.5:1 to about 1.0:1, or from about 1.0:1 to about 0.5:1, or from about 1:0.5 to about 1:1.0, or from about 1:1.0 to about 1:1.5, or from about 1:1.5 to about 1:2.0, or from about 1:2.0 to about 1:2.5, or from about 1:2.5 to about 1:3.0, or from about 1:3.0 to about 1:3.5, or from about 1:3.5 to about 1:4.0, or from about 1:4.0 to about 1:4.5, or from about 1:4.5 to about 1:4.0. In other embodiments, the mass ratio of the peptide to the additive ranges from about 1.0 to about 1.5.

In some embodiments, a pH of the reconstituted aqueous composition ranges from about 5 to about 12. In certain embodiments, the pH of the reconstituted aqueous composition ranges from about 5 to about 12, or from about 5 to about 6, or from about 6 to about 7, or from about 7.0 to about 7.1, or from about 7.1 to about 7.2, or from about 7.2 to about 7.3, or from about 7.3 to about 7.4, or from about 7.4 to about 7.5, or from about 7.5 to about 7.6, or from about 7.6 to about 7.7, or from about 7.7 to about 7.8, or from about 7.8 to about 7.9, or from about 7.9 to about 8.0, or from about 8.0 to about 8.1, or from about 8.1 to about 8.2, or from about 8.2 to about 8.3, or from about 8.3 to about 8.4, or from about 8.4 to about 8.5, or from about 8.5 to about 8.6, or from about 8.6 to about 8.7, or from about 8.7, to about 8.8, or from about 8.8 to about 8.9, or from about 8.9 to about 9.0, or from about 9 to about 10, or from about 10 to about 11, of from about 11 to about 12. In certain embodiments, the pH of the reconstituted aqueous composition ranges from about 7.2 to about 7.8.

In some embodiments, a concentration of the peptide (i.e., Bevonescein (ALM-488)) in the reconstituted aqueous composition ranges from about 10 mg/mL to about 500 mg/mL based on a total volume of the aqueous composition. In certain embodiments, a concentration of the peptide in the reconstituted aqueous composition ranges from about 20 mg/mL to about 30 mg/mL, or from about 30 mg/mL to about 40 mg/mL, or from about 40 mg/mL to about 50 mg/mL, or from about 50 mg/mL to about 60 mg/mL, or from about 60 mg/mL to about 70 mg/mL, or from about 70 mg/mL to about 80 mg/mL, or from about 80 mg/mL to about 90 mg/mL, or from about 90 mg/mL to about 100 mg/mL, or from about 100 mg/mL to about 110 mg/mL, or from about 110 mg/mL to about 120 mg/mL, or from about 120 mg/mL to about 130 mg/mL, or from about 130 mg/mL to about 140 mg/mL, or from about 140 mg/mL to about 150 mg/mL, or from about 150 mg/mL to about 160 mg/mL, or from about 160 mg/mL to about 170 mg/mL, or from about 170 mg/mL to about 180 mg/mL, or from about 180 mg/mL to about 190 mg/mL or from about 190 mg/mL to about 200 mg/mL, or from about 200 mg/mL to about 250 mg/mL, or from about 250 mg/mL to about 300 mg/mL, or from about 300 mg/mL to about 350 mg/mL, or from about 350 mg/mL to about 400 mg/mL, or from about 400 mg/mL to about 450 mg/mL, based on a total volume of the aqueous composition. In certain embodiments, a concentration of the peptide in the reconstituted aqueous composition ranges from about 25 mg/mL to about 200 mg/mL based on the total volume of the aqueous composition. In other embodiments, a concentration of the peptide in the reconstituted aqueous composition ranges from about 40 mg/mL to about 75 mg/mL based on the total volume of the aqueous composition.

In some embodiments, a density of the reconstituted aqueous composition ranges from about 0.6 g/mL to about 1.3 g/mL. In certain embodiments the density of the reconstituted aqueous compositions ranges from about 0.6 g/mL to about 0.7 g/mL, or from about 0.7 g/mL to about 0.8 g/mL, or from about 0.8 g/mL to about 0.85 g/mL, or from about 0.85 g/mL to about 0.90 g/mL, or from about 0.90 g/mL to about 0.95 g/mL, or from about 0.95 g/mL to about 1.0 g/mL, or from about 1.0 g/mL to about 1.05 g/mL, or from about 1.05 g/mL to about 1.10 g/mL, or from about 1.10 g/mL to about 1.15 g/mL, or from about 1.15 g/mL to about 1.20 g/mL, or from about 1.20 g/mL to about 1.25 g/mL. In other embodiments, the density of the reconstituted aqueous composition ranges from about 1.0 g/mL to about 1.08 g/mL.

In some embodiments, an osmolality of the reconstituted aqueous composition ranges from about 150 mOsm/kg to about 400 mOsm/kg. In certain embodiments the osmolality of the reconstituted aqueous composition ranges from about 150 mOsm/kg to about 175 mOsm/kg, or from about 175 mOsm/kg to about 200 mOsm/kg, or from about 200 mOsm/kg to about 225 mOsm/kg, or from about 225 mOsm/kg to about 250 mOsm/kg, or from about 250 mOsm/kg to about 275 mOsm/kg, or from about 275 mOsm/kg to about 300 mOsm/kg, or from about 300 mOsm/kg to about 325 mOsm/kg, or from about 325 mOsm/kg to about 350 mOsm/kg. In other embodiments the osmolality of the reconstituted aqueous composition ranges from about 280 mOsm/kg to about 300 mOsm/kg.

In some embodiments, a volume of the reconstituted aqueous composition ranges from about 0.5 mL to about 5.0 mL. In certain embodiments, the volume the reconstituted aqueous composition ranges from about 0.5 mL to about 1.0 mL, or from about 1.0 mL to about 1.5 mL, or from about 1.5 mL to about 2.0 mL, or from about 2.0 mL to about 2.5 mL, or from about 2.5 mL to about 3.0 mL, or from about 3.0 mL to about 3.5 mL, or from about 3.5 mL to about 4.0 mL, or from about 4.0 mL to about 4.5 mL. In other embodiments the volume of the reconstituted aqueous composition ranges from about 1.8 mL to about 2.2 mL.

In some embodiments, the reconstituted aqueous composition comprises at least 95 mass % of the peptide (i.e., Bevonescein (ALM-488)), as measured relative to a total mass of the reconstituted aqueous composition, after storage at 40° C. for a period of 14 days. In certain embodiments, the reconstituted aqueous composition comprises at least 96.0 mass %, or at least 96.5 mass %, or at least 97.0 mass %, or at least 97.5 mass %, or at least 98.0 mass %, or at least 98.5 mass %, or at least 99.0 mass %, or at least 99.5 mass %, of the peptide, as measured relative to a total mass of the reconstituted aqueous composition, after storage at 40° C. for a period of 14 days.

Another embodiment relates to methods of forming a reconstituted aqueous composition by combining a lyophilized composition disclosed herein with an aqueous vehicle.

In some embodiments, the method includes stirring or agitating a combined mixture of the lyophilized composition and the aqueous vehicle to obtain the reconstituted aqueous composition.

In some embodiments, the method includes heating the combined mixture.

In some embodiments, the lyophilized composition used to prepare the reconstituted aqueous composition is in the form of an elegant lyophilized cake.

### Methods of Use

Another embodiment relates to methods for labeling or identifying a neuron or nerve by contacting the neuron or nerve with an Bevonescein (ALM-488)-containing composition disclosed herein. In some embodiments, the neuron or nerve is contacted with a free fluorescent moiety in combination with the composition. In some embodiments, the fluorescent moiety comprises a fluorescent protein, a fluorescent peptide, a fluorophore, or any combination thereof. In some embodiments, the contacting occurs in vivo. In some embodiments, the contacting occurs in vitro. In some embodiments the neuron or nerve is a human neuron or nerve.

In some embodiments, a nerve targeting peptide ALM-488 is administered in combination (simultaneously, concurrently, or serially) with a fluorescent moiety (e.g., fluorescent moiety that is not conjugated to the nerve target peptide conjugate, or “free” fluorescent moiety). In some embodiments, the fluorescent moiety is a fluorescein, e.g., carboxyfluorescein.

Another embodiment relates to methods for delivering at least one drug to a neuron or nerve by contacting the neuron or nerve with an ALM-488-containing composition disclosed herein. In some embodiments, the delivery of the at least one drug to the neuron or nerve is carried out by administering the composition to a subject in need thereof. In some embodiments, the subject is a human subject. In certain embodiments, the composition is administered to the subject prior to performing a surgical procedure on the subject. In some embodiments the surgical procedure is a cancer surgical procedure such as, for example, a prostate cancer surgical procedure.

In some embodiments of the methods described herein, the nerve targeting peptide ALM-488 is administered to a subject, as described herein, including humans and mammals (e.g., mice, rats, pigs, cats, dogs, and horses). In some embodiments, subjects are mammals. In some embodiments, subjects are primates. In some embodiments, subjects are humans. In some embodiments, human subjects are pediatric subjects (age 21 years and younger), adult subjects (age 22 years to 65 years), or geriatric subjects (age 65 years and above).

In some embodiments of the methods of use described herein (e.g., labeling or identifying a neuron or nerve, delivering drug(s) to a neuron or nerve) may be carried out before or during surgery on the subject. In some embodiments, the surgery is related to a traumatic injury in the subject. In some embodiments, the surgery is related to an infection in the subject. In some embodiments, the surgery is plastic surgery e.g., cosmetic or reconstructive surgery, in the subject.

In some embodiments, the surgery is head, neck, skull base, spinal, prostate, heart, kidney, hand, arm, foot, leg, lower abdominal, or gynecological surgery. In some embodiments, the surgery is head and neck surgery. In some embodiments, the surgery is gastrointestinal tract (GI) surgery or genitourinary tract (GU) surgery.

In some embodiments the surgery is cancer surgery. In some embodiments the cancer is selected from the group consisting of prostate cancer, liver cancer (HCC), colorectal cancer, ovarian cancer, endometrial cancer, breast cancer, pancreatic cancer, stomach cancer, cervical cancer, head and neck cancer, thyroid cancer, testis cancer, urothelial cancer, lung cancer, melanoma, testicular germ cell tumors, mesothelioma, and esophageal cancer. In some embodiments, the cancer is prostate cancer.

In some embodiments, the method comprises administering an ALM-488-containing composition disclosed herein to a subject that will undergo surgery. In some embodiments, the method comprises administering to a subject that is currently undergoing surgery. In some embodiments, an ALM-488-containing composition disclosed herein is administered to a patient systemically, for example, by intravenous injection. In some embodiments, a nerve targeting peptide conjugate disclosed herein is administered to a patient locally.

In some embodiments, nerve targeting peptide ALM-488 is delivered to a subject via a drug delivery vehicle or carrier. In some embodiments, a delivery vehicle is made from natural or synthetic materials or both. In some embodiments, a delivery vehicle may include a nanoparticle, microparticle, polymeric micelle, nanocapsule, dendrimer, large PEG, nanogel, liposome, fullerene, nanostructured lipid carrier, nanoshell, quantum dot, protein-based nanocarriers (e.g., albumin, elastin, gliadin, legumin, zein, soy protein, milk protein, whey based nanocarriers), organic nanocarrier (e.g., gelatin, dextran, guar gum, chitosan, collagen), polysaccharide based carrier (e.g., dextran, chitosan, pectin), lipid emulsion, or a combination thereof.

In certain embodiments, an ALM-488-containing composition disclosed herein is administered to a subject by any suitable administration route, including but not limited to, topical, oral, intrarectal, intravaginal, intranasal, inhalation, parenteral (intravenous, intraarterial, subcutaneous, intraperitoneal, intramuscular, intravascular, intrathecal, intravitreal, infusion) administration. In certain embodiments, a pharmaceutical composition disclosed herein is administered to a subject is administered locally or systemically. In embodiments, a pharmaceutical composition disclosed herein is administered intravenously such as, for example, by systemic intravenous injection.

Formulations suitable for intramuscular, subcutaneous, or intravenous injection include physiologically acceptable sterile aqueous or non-aqueous solutions, dispersions, suspensions or emulsions, and sterile powders (such as lyophilized compositions described herein) for reconstitution into sterile injectable solutions or dispersions. Examples of suitable aqueous and non-aqueous carriers, diluents, solvents, or vehicles including water, ethanol, polyols (propylene glycol, polyethylene-glycol, glycerol, cremophor and the like), suitable mixtures thereof, vegetable oils (such as olive oil) and injectable organic esters such as ethyl oleate. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation. Bioavailability enhancers may include penetration or permeation enhancers. See, e.g., Muheem et al., 2016, Saudi Pharm. J. 24, 413-428; Brayden et al. 2020, Adv Drug Deliv Rev. 157, 2-36; Ibrahim et al. 2020, J. Pham. Sci. 28, 403-416. In some formulations, proper fluidity can be maintained, for example, by the use of a coating such as lecithin, by the maintenance of the required particle size in the case of dispersions, and by the use of surfactants. Formulations suitable for subcutaneous injection also contain optional additives such as preserving, wetting, emulsifying, and dispensing agents, as are known in the art. In embodiments, formulations suitable for intravenous injection may be prepared in aqueous solutions, such as saline buffers and other physiologically compatible buffers known in the art.

Parenteral injections optionally involve bolus injection or continuous infusion. Formulations for injection are optionally presented in unit dosage form, e.g., in ampoules or in multi dose containers, with an added preservative. In some embodiments, the ALM-488-containing compositions described herein are in a form suitable for parenteral injection as sterile suspensions, solutions or emulsions in oily or aqueous vehicles, and contain formulating agents such as suspending, stabilizing and/or dispersing agents. Pharmaceutical formulations for parenteral administration include aqueous solutions of an active agent in water soluble form. Additionally, suspensions are optionally prepared (from, for example, lyophilized compositions disclosed herein) as appropriate oily injection suspensions.

In some embodiments, an ALM-488-containing composition disclosed herein may be administered orally. Dosage forms suitable for oral administration can be solid or liquid and may include for example, a pill, capsule, troche, tablet, caplet, gel caplet (gel cap), syrup, an aqueous suspension or solution, a chewable form, a swallowable form, a dissolvable form, an effervescent, a granulated form, and an oral liquid solution. In a specific embodiment, the dosage form is a solid dosage form, and more specifically, comprises a tablet or capsule, or is orally administered by a vehicle or carrier as disclosed herein.

In some embodiments, the ALM-488-containing pharmaceutical compositions described herein are in unit dosage forms suitable for single administration of precise dosages. In unit dosage form, the formulation is divided into unit doses containing appropriate quantities of an active agent disclosed herein. In some embodiments, the unit dosage is in the form of a package containing discrete quantities of the formulation. Non-limiting examples are packaged tablets or capsules, and powders in vials or ampoules. In some embodiments, aqueous suspension compositions are packaged in single-dose non-reclosable containers. Alternatively, multiple-dose reclosable containers are used, in which case it is typical to include a preservative in the composition. By way of example only, formulations for parenteral injection are presented in unit dosage form, which include, but are not limited to ampoules, or in multi dose containers, with an added preservative.

As explained above, certain embodiments of the present disclosure relate to methods of targeted drug delivery. In some embodiments, an ALM-488-containing composition disclosed herein delivers a drug to a specific target. In some embodiments, the composition delivers a drug to a neuron or nerve.

In some embodiments, the drug is an agent that reduces pain (either the perception of pain or activity of a painful stimulant). In some embodiments, the drug is an anesthetic. In some embodiments, the drug is benzocaine; carticaine; cinchocaine; cyclomethycaine; lidocaine; prilocaine; propxycaine; proparacaine; tetracaine; tocainide; and trimecaine; or a combination thereof.

In some embodiments, the drug is an agent that modulates death (e.g., via apoptosis or necrosis) of a neuron or nerve. In some embodiments, the drug is a cytotoxic agent. In some embodiments, the drug is methotrexate (RHEUMATREX®, Amethopterin); cyclophosphamide (CYTOXAN®); thalidomide (THALIDOMID®); paclitaxel; pemetrexed; pentostatin; pipobroman; pixantrone; plicamycin; procarbazine; proteasome inhibitors (e.g.; bortezomib); raltitrexed; rebeccamycin; rubitecan; SN-38; salinosporamide A; satraplatin; streptozotocin; swainsonine; tariquidar; taxane; tegafur-uracil; temozolomide; testolactone; tbioTEPA; tioguanine; topotecan; trabectedin; tretinoin; triplatin tetranitrate; tris(2-chloroethyl)amine; troxacitabine; uracil mustard; valrubicin; vinblastine; vincristine; vinorelbine; vorinostat; zosuquidar; or a combination thereof. In some embodiments, the drug is a pro-apoptotic agent. In some embodiments, the drug is an anti-apoptotic agent. In some embodiments, the drug is selected from minocycline; SB-203580 (4-(4-Fluorophenyl)-2-(4-methylsulfmyl phenyl)-5-(4-pyridyl) 1H-imidazole); PD 169316 (4-(4-Fluorophenyl)-2-(4-nitrophenyl)-5-(4-pyridyl)-1H-imidazole); SB 202190 (4-(4-Fluorophenyl)-2-(4-hydroxyphenyl)-5-(4-pyridyl)1H-imidazole); RWJ 67657 (4-[4-(4-fluorophenyl)-1-(3-phenylpropyl)-5-(4-pyridinyl)-1H-imidazol-2-yl]-3-butyn-1-ol); SB 220025 (5-(2-Amino-4-pyrimidinyl)-4-(4-fluorophenyl)-1-(4-piperidinlyl) imidazole); D-JNKI-1 ((D)-hJIP 175_i 57-DPrO-DPrO-(D)-HIV-TAT57-48); AM-111 (Auris); SP600125 (anthra[1,9-cd]pyrazol-6 (2H)-one); JNK Inhibitor I ((L)-HIV-T AT48-57-PP-JBD20); JNK Inhibitor III ((L)-HIV-TAT47-57-gaba-c-Jun833-57); AS601245 (1,3-benzothiazol-2-yl(2-[[2-(3-pyridinyl)ethyl] amino]-4 pyrimidinyl) acetonitrile); JNK Inhibitor VI (H2N-RPKRPTTLNLF-NH2); JNK Inhibitor VIII (N-(4-Amino-5-cyano-6-ethoxypyridin-2-yl)-2-(2,5-dimethoxyphenyl) acetamide); JNK Inhibitor IX (N-(3-Cyano-4,5,6,7-tetrahydro-1-benzothien-2-yl)-1-naphthamide); dicumarol (3,3′-Methylenebis(4-hydroxycoumarin)); SC-236 (4-[5-(4-chlorophenyl)-3-(trifluoromethyl)-1H-pyrazol-1-yl]benzene-sulfonamide); CEP-1347 (Cephalon); CEP-11004 (Cephalon); an artificial protein comprising at least a portion of a Bcl-2 polypeptide; a recombinant FNK; V5 (also known as Bax inhibitor peptide V5); Bax channel blocker ((+)-1-(3,6-Dibromocarbazol-9-yl)-3-piperazin-1-yl-propan-2-ol); Bax inhibiting peptide P5 (also known as Bax inhibitor peptide P5); Kp7-6; FAIM(S) (Fas apoptosis inhibitory molecule-short); FAIM (L) (Fas apoptosis inhibitory molecule-long); Fas: Fc; FAP-1; NOK2; F2051; F1 926; F2928; ZB4; Fas M3 mAb; EGF; 740 Y-P; SC 3036 (KKHTDDGYMPMSPGVA); PI 3-kinase Activator (Santa Cruz Biotechnology, Inc.); Pam3Cys ((S)-(2,3-bis(palmitoyloxy)-(2RS)-propyl)-N-palmitoyl-(R)-Cys-(S)-Ser(S)-Lys4-OH, trihydrochloride); Actl (NF-kB activator 1); an anti-DcB antibody; Acetyl-11-keto-b-Boswellic Acid; Andrographolide; Caffeic Acid Phenethyl Ester (CAPE); Gliotoxin; Isohelenin; NEMO-Binding Domain Binding Peptide (DRQIKIWFQNRRMKWKKTALDWSWLQTE); NF-kB Activation Inhibitor (6-Amino-4-(4-phenoxyphenylethylamino) quinazoline); NF-κB Activation Inhibitor II (4-Methyl-Nl-(3-phenylpropyl)benzene-1,2-diamine); NF-κB Activation Inhibitor III (3-Chloro-4-nitro-N-(5-nitro-2-thiazolyl)-benzamide); NF-κB Activation hihibitor IV ((E)-2-Fluoro-4′-methoxystilbene); NF-κB Activation Inhibitor V (5-Hydroxy-(2,6-diisopropylphenyl)-1H-isoindole-1,3-dione); NF-κB SN50 (AA VALLP A VLLALLAP VQRKRQKLMP); Oridonin; Parthenolide; PPM-18 (2-Benzoylamino-1,4-naphthoquinone); Rol06-9920; Sulfasalazine; TIRAP Inhibitor Peptide (RQIKIWFNRRMKWKKLQLRD AAPGG AIVS); Withaferin A; Wogonin; BAY 11-7082 ((E) 3-[(4-Methylphenyl) sulfonyl]-2-propenenitrile); BAY 11-7085 ((E) 3-[(4-t-Butylphenyl) sulfonyl]-2-propenenitrile); (E)-Capsaicin; Aurothiomalate (ATM or AuTM); Evodiamine; Hypoestoxide; IKK Inhibitor III (BMS-345541); IKK Inhibitor VII; IKK Inhibitor X; IKK Inhibitor II; IKK-2 Inhibitor IV; IKK-2 Inhibitor V; IKK-2 Inhibitor VI; IKK-2 Inhibitor (SC-514); IkB Kinase Inhibitor Peptide; IKK-3 Inhibitor LX; ARRY-797 (Array BioPharma); SB-220025 (5-(2-Amino-4-pyrimidinyl)-4-(4-fluorophenyl)-1-(4-piperidinlyl) imidazole); SB-239063 (trans-4-[4-(4-Fluorophenyl)-5-(2-methoxy-4-pyrimidinyl)-1H-imidazol-1-yl]cyclo-hexanol); SB-202190 (4-(4-Fluorophenyl)-2-(4-hydroxyphenyl)-5-(4-pyridyl) 1H-imidazole); JX-401 ([2-Methoxy-4-(methylthio)benzoyl]-4-(phenylmethyl) piperidine); PD-169316 (4-(4-Fluorophenyl)-2-(4-nitrophenyl)-5-(4-pyridyl)-1H-imidazole); SKF-86002 (6-(4-Fluorophenyl)-2,3-dihydro-5-(4-pyridinyl) imidazo[2,1-b]thiazole dihydrochloride); SB-200646 (N-(1-Methyl-1H-indol-5-yl)-N′-3-pyridinylurea); CMPD-I (2′-Fluoro-N-(4-hydroxyphenyl)-[1,1′-biphenyl]-4-butanamide); EO-1428 ((2-Methylphenyl)-[4-[(2-amino-4-bromophenyl)amino]-2-chlorophenyl]methanone); SB-253080 (4-[5-(4-Fluorophenyl)-2-[4-(methylsulfonyl)phenyl]-1H-imidazol-4-yl]pyridine); SD-169 (1H-Indole-5-carboxamide); SB-203580 (4-(4-Fluorophenyl)-2-(4-methylsulfomyl phenyl)-5-(4-pyridyl)-1H-imidazole); TZP-101 (Tranzyme Pharma); TZP-102 (Tranzyme Pharma); GHRP-6 (growth hormone-releasing peptide-6); GHRP-2 (growth hormone-releasing peptide-2); EX-1314 (Elixir Pharmaceuticals); MK-677 (Merck); L-692,429 (Butanamide, 3-amino-3-methyl-N-(2,3,4,5-tetrahydro-2-oxo-1-((2′-(1H-tetrazol-5-yl)(1,1′-biphenyl)-4-yl)methyl)-1H-1-benzazepin-3-yl)-, (R)-); EP1572 (Aib-DTrp-DgTφ-CHO); diltiazem; metabolites of diltiazem; BRE (Brain and Reproductive organ-Expressed protein); verapamil; nimodipine; diltiazem; omega-conotoxin; GVIA; amlodipine; felodipine; lacidipine; mibefradil; NPPB (5-Nitro-2-(3-phenylpropylamino)benzoic Acid); flunarizine; erythropoietin; piperine; hemin; brazilin; z-V AD-FMK (Benzyloxycarbonyl-Val-Ala-Asp (OMe)-fluoromethylketone); z-LEHD-FMK (benzyloxycarbonyl-Leu-Glu (OMe)-His-Asp (OMe)-fluoromethylketone); B-D-FMK (boc-aspartyl (Ome)-fluoromethylketone); Ac-LEHD-CHO (N-acetyl-Leu-Glu-His-Asp-CHO); Ac-IETD-CHO (N-acetyl-Ile-Glu-Thr-Asp-CHO); z-IETD-FMK (benzyloxycarbonyl-Ile-Glu (OMe)-Thr-Asp (OMe)-fluoromethy lketone); FAM-LEHD-FMK (benzyloxycarbonyl Leu-Glu-His-Asp-fluoromethyl ketone); FAM-LETD-FMK (benzyloxycarbonyl Leu-Glu-Thr-Asp-fluoromethyl ketone); Q-VD-OPH (Quinoline-Val-ASp-CH2-O-Ph); XIAP; cIAP-1; cIAP-2; ML-IAP; ILP-2; NAIP; Survivin; Brace; IAPL-3; fortilin; leupeptine; PD-150606 (3-(4-Iodophenyl)-2-mercapto-(Z)-2-propenoic acid); MDL-28170 (Z-Val-Phe-CHO); calpeptin; acetyl-calpastatin; MG 132 (N-[(phenylmethoxy) carbonyl]-L-leucyl-N-[(1S)-1-formyl-3-methylbutyl]-L-leucinamide); MYODUR; BN 82270 (Ipsen); BN 2204 (Ipsen); AHLi-11 (Quark Pharmaceuticals), an mdm2 protein, pifithrin-α(1-(4-Methylphenyl)-2-(4,5,6,7-tetrahydro-2-imino-3 (2H)-benzothiazolyl) ethanone); trans-stilbene; cis-stilbene; resveratrol; piceatannol; rhapontin; deoxyrhapontin; butein; chalcon; isoliquirtigen; butein; 4,2′,4′-trihydroxychalcone; 3,4,2′,4′,6′-pentahydroxychalcone; flavone; morin; fisetin; luteolin; quercetin; kaempferol; apigenin; gossypetin; myricetin; 6-hydroxyapigenin; 5-hydroxyflavone; 5,7,3′,4′,5′-pentahydroxyflavone; 3,7,3′,4′,5′-pentahydroxyflavone; 3,6,3′,4′-tetrahydroxyflavone; 7,3′,4′,5′-tetrahydroxyflavone; 3,6,2′,4′-tetrahydroxyflavone; 7,4′-dihydroxyflavone; 7,8,3′,4′-tetrahydroxyflavone; 3, 6,2′,3 ‘-tetrahydroxyflavone; 4’-hydroxyflavone; 5-hydroxyflavone; 5,4′-dihydroxyflavone; 5,7-dihydroxyflavone; daidzein; genistein; naringenin; flavanone; 3,5,7,3′,4′-pentahydroxyflavanone; pelargonidin chloride; cyanidin chloride; delphinidin chloride; (−)-epicatechin (Hydroxy Sites: 3,5,7,3′,4′; (−)-catechin (Hydroxy Sites: 3,5,7,3′,4); (−)-gallocatechin (Hydroxy Sites: 3,5,7,3 ‘,4’,5) (+)-catechin (Hydroxy Sites: 3,5,7,3′,4′; (+)-epicatechin (Hydroxy Sites: 3,5,7,3′,4′; Hinokitiol (b-Thujaplicin; 2-hydroxy-4-isopropyl-2,4,6-cycloheptatrien-1-one); L-(+)-Ergothioneine ((S)-a-Carboxy-2,3-dihydro-N,N,N-trimethyl-2-thioxo-1H-iniidazole4-ethanaminium inner salt); Caffeic Acid Phenyl Ester; MCI-186 (3-Methyl-1-phenyl-2-pyrazolin-5-one); HBED (N,N′-Di-(2-hydroxybenzyl)ethylene-diamine-N,N′-diaceticacid*H2O); Ambroxol (trans-4-(2-Amino-3,5-dibromobenzylamino)-cyclohexane-HCl; and U-83836E ((−)-2-((4-(2,6-di-1-Pyrrolidinyl-4-pyrimidinyl)-1-piperzainyl)-methyl)-3,4-dihydro-2,5,7,8-tetramethyl-2H-1-benzopyran-6-ol»2HCl); β-1-5-methyl-nicotin-amide-2′-deoxyribose; 3-D-1′-5-methyl-nico-tinamide-2′-deoxyribofuranoside; 3-1′-4,5-dimethyl-nicotinamide-2′-de-oxyribose; 3-D-1′-4,5-dimethyl-nicotmamide-2′-deoxyribofuranoside; 1-Naphthyl PP1 (1-(1,1-Dimethylethyl)-3-(1-naphthalenyl)-1H-pyrazolo[3,4-d]pyrimidin-4-amine); Lavendustin A (5-[(2,5-Dihydroxyphenyl)methyl][(2-hydroxyphenyl) methy 1]amino]-2-hydroxy-benzoic acid); MNS (3,4-Methylenedioxy-b-nitrostyrene); PP1 (1-(1,1-Dimethylethyl)-1-(4-methylphenyl)-1H-pyrazolo[3,4-d]pyrimidin-4-amine); PP2 (3-(4-chlorophenyl)-1-(1,1-dimethylethyl)-1H-pyrazolo[3,4-d]pyrimidin-4-amine); KX1-004 (Kinex); KX1-005 (Kinex); KX1-136 (Kinex); KX1-174 (Kinex); KX1-141 (Kinex); KX2-328 (Kinex); KX1-306 (Kinex); KX1-329 (Kinex); KX2-391 (Kinex); KX2-377 (Kinex); ZD4190 (Astra Zeneca; N-(4-bromo-2-fluorophenyl)-6-methoxy-7-(2-(1H-1,2,3-triazol-1-yl) ethoxy) quinazolin-4-amine); AP22408 (Ariad Pharmaceuticals); AP23236 (Ariad Pharmaceuticals); AP23451 (Ariad Pharmaceuticals); AP23464 (Ariad Pharmaceuticals); AZD0530 (Astra Zeneca); AZM475271 (M475271; Astra Zeneca); Dasatinib (N-(2-chloro-6-methylphneyl)-2-(6-(4-(2-hydroxyethyl)-piperazin-1-yl)-2-methylpyrimidin-4-ylamino) thiazole-5-carboxamide); GN963 (trans-4-(6,7-dimethoxy-quinoxalin-2ylamino)cyclohexanolsulfate); Bosutinib (4-((2,4-dichloro-5-methoxyphenyl)-amino)-6-methoxy-7-(3-(4-methyl-1-piperazinyl) propoxy)-3-quinolinecarbonitrile); or combinations thereof.

In some embodiments, the drug is an agent that reduces undesired neuron or nerve impulses. In some embodiments, the drug reduces one or more symptoms of dyskinesia or synkinesia. In some embodiments, the drug is carbamazepine, oxcarbazepine, phenytein, valproic acid, sodium valproate, cinnarizine, flunarizine, or nimodipine, or combinations thereof.

In some embodiments, the drug is an agent that promotes regeneration of neuron or nerve tissue. In some embodiments, the drug is a growth factor. In some embodiments, the drug is selected from brain-derived neurotrophic factor (BDNF); ciliary neurotrophic factor (CNTF); glial cell-line derived neurotrophic factor (GDNF); neurotrophin-3; neurotrophin-4; fibroblast growth factor (FGF) receptor; insulin-like growth factor (IGF); or a combination thereof.

Disclosed herein, in certain embodiments, are methods of delivering a photosensitizing agent to a human neuron or nerve comprising contacting the human neuron or nerve with an ALM-488-containing composition disclosed herein. In some embodiments, the method further comprises exposing the human neuron or nerve with a light source that activates the photosensitizing agent contained in ALM-488, wherein the activated photosensitizing agent induces ablation or killing of the human neuron or nerve. Upon exposure to a specific wavelength of light, a photosensitizing agent reacts with molecular oxygen to produce singlet oxygen, which is cytotoxic.

In some embodiments, ALM-488-containing compositions disclosed can be used for treating prostate cancer in a subject. Autonomic innervation may contribute to prostate cancer growth and metastasis by light induced ablation of local autonomic nerves. Thus, local autonomic nerves may be a viable target for prostate cancer therapy. In some embodiments, renovascular hypertension can be treated in a subject by light induced ablation of sympathetic nerves in the renal vessels. In some embodiments, ALM-488-containing compositions disclosed can be used for treating excessive sweating. In some embodiments, ALM-488-containing compositions disclosed can be used for treating cardiac arrhythmias. In some embodiments, ALM-488-containing compositions disclosed can be used for treating pathological muscle spasms (e.g., Meige syndrome, hemifacial spasm, torticollis).

In some embodiments, an ALM-488-containing composition disclosed herein may be administered in combination (simultaneously, concurrently, or serially) with a second nerve targeting peptide conjugate.

Another embodiment relates to use of an ALM-488-containing composition disclosed herein in the preparation of a medicament for labeling or identifying a neuron or nerve in a subject. Another embodiment relates to an ALM-488-containing composition disclosed herein which is used to label or identify a neuron or nerve in a subject. Another embodiment relates to use of an ALM-488-containing composition disclosed herein in the preparation of a medicament for delivering at least one drug to a neuron or nerve in a subject. Another embodiment relates to an ALM-488-containing composition disclosed herein which is used to deliver at least one drug to a neuron or nerve in a subject.

While aspects of the present disclosure have been shown and described throughout this disclosure, it will be apparent to those skilled in the art that such aspects are provided by way of example only. Numerous variations, changes, and substitutions will now occur to those skilled in the art without departing from the disclosure. It should be understood that various alternatives to the aspects of the disclosure described throughout this disclosure may be employed in practicing the disclosure. It is intended that the following claims define the scope of the disclosure and that methods and structures within the scope of these claims and their equivalents be covered thereby.

The various embodiments described above can be combined to provide some embodiments. All of the U.S. patents, U.S. patent application publications, U.S. patent applications, foreign patents, foreign patent applications and non-patent publications referred to in this specification and/or listed in the Application Data Sheet, are incorporated herein by reference, in their entirety. Aspects of the embodiments can be modified, if necessary to employ concepts of the various patents, applications and publications to provide yet some embodiments.

### EXAMPLES

### Abbreviations

Ac₂O: acetic anhydride;
ACN: acetonitrile;
DIC: N,N′-diisopropylcarbodiimide;
DIEA: N,N-Diisopropylethylamine;
DMF: N,N-dimethylformamide;
DSC: differential scanning calorimetry;
EDT: ethane-1,2-dithiol;
5-FAM: 5-carboxyfluorescein;
Fmoc: 9-fluorenylmethyloxycarbonyl;
FDM: freeze-drying microscopy;
HBTU: N,N,N′,N′-tetramethyl-O-(1H-benzotriazol-1-yl) uronium hexafluorophosphate;
HNS: half-normal saline;
HOBt: Hydroxybenzotriazole;
HPLC: high-performance (high-pressure) liquid chromatograhy;
MBHA: 4-methylbenzhydrylamine;
MeOH: methanol;
NS: normal saline;
Pip: piperidine;
PVDF: polyvinylene fluoride;
RP: reverse phase;
SPPS: solid-phase peptide synthesis;
TFA: trifluoroacetic acid;
TIS: triisopropylsilane;
UPLC: ultra-performance liquid chromatography;

### Instruments

The instruments used herein are set forth in Table 1.

TABLE 1
Instruments utilized

| Instrument | Make/Model/Serial Number |
|---|---|
| Branson 5800 Ultrasonic Bath | Branson Ultrasonics Corp. |
| | Model CPX5800H |
| IKA magnetic stirrer | Model RO 10 S000 |
| Radiometer MeterLab pH/ION Meter | Model PHM240 |
| Fisher Scientific, Isotemp ® Oven | Model #630F |
| Freeze-drying microscope | BioPharma Technology Limited Lyostat2 |
| Virtis Advantage XL freeze dryer | The Virtis Company (SP Scientific) |
| Agilent 1100 HPLC system | G1312A, Binary Pump w Degasser |
| | G1329A Auto sampler |
| | G1330B FC/ALS Therm (1200 series) |
| | G1316A Column heater |
| | G13658 Multi Wavelength detector |
| Differential scanning calorimeter | Model Q20 with RCS 90 |
| Bruker X-ray Diffractometer | Model D8 Discover |
| Mettler Toledo Karl Fischer Coulometer | Model DL 39 with Mettler Toledo Stromboli |
| | automatic oven sample changer |
| 40° C./75% RH Stability Chamber | Model: ES 2000 Reach-in |
| 25° C./60% RH Stability Chamber | Norlake Scientific |
| Advance Osmometer | Model: 3320 |
| Perkin Elmer Thermogravimetric | Model: TGA 7 |
| Analyser (TGA 7) | |
| 40° C./75% RH Stability Chamber | Model: ES 2000 Reach-in |
| 25° C./60% RH Stability Chamber | Norlake Scientific |

### Materials

The materials used herein are set forth in Table 2 below.

TABLE 2
Materials used

| Material | Details |
|---|---|
| Acetonitrile (HPLC Grade) | Sigma-Aldrich |
| Sodium Chloride, USP | SAFC |
| Sterile Water For Injection | Baxter |
| Trifluoroacetic Acid, LC Grade | Sigma |
| HPLC Grade Water | Sigma-Aldrich |
| Sodium Hydroxide pellets, (NF) | Spectrum Chemicals |
| Meglumine, USP | Spectrum Chemicals |
| L-Arginine (Free Base) USP | Spectrum Chemicals |
| Glycine, USP | Spectrum Chemicals, |
| Mannitol, USP | Spectrum Chemicals |
| Trehalose Dihydrate | Spectrum Chemicals |
| Hydrochloric Acid Solution, 1N | Fisher Scientific |
| Hydrochloric Acid Solution, 0.1N | Fisher Scientific |
| Tromethamine, USP | Spectrum Chemicals |
| Sodium Hydroxide Solution, 0.1N | Fisher Scientific |
| Sodium Hydroxide Solution, 1N | Fisher Scientific |

### HPLC Conditions

The HPLC conditions for analyzing the purity of ALM-488 are summarized in Table 3 below.

TABLE 3
HPLC method conditions

| Column | Phenomenex Luna Plus C18 (2) | | |
|---|---|---|---|
| | 100 Å, 4.6 × 250 mm, 5 μm | | |
| Detection Wavelength | 214 nm | | |
| Column Temp. | 30 ± 2° C. | | |
| Flow Rate | 1.0 mL/min | | |
| Nominal Injection Volume | 15 μL | | |
| Typical. solution concn. | ~0.250 mg/mL | | |
| Sample diluent | Water, MPA | | |
| Sample Temperature | 5 ± 1° C. | | |
| Mobile Phase A, MPA | 0.1% TFA in Water | | |
| Mobile Phase B, MPB | 0.1% TFA in Acetonitrile | | |
| | Time | % MP-A | % MP-B |
| Mobile Phase gradient | 0.0 min | 75 | 25 |
| | 1.0 min | 75 | 25 |
| | 20.0 min | 45 | 55 |
| | 20.1 min | 75 | 25 |
| | 27.0 min | 75 | 25 |

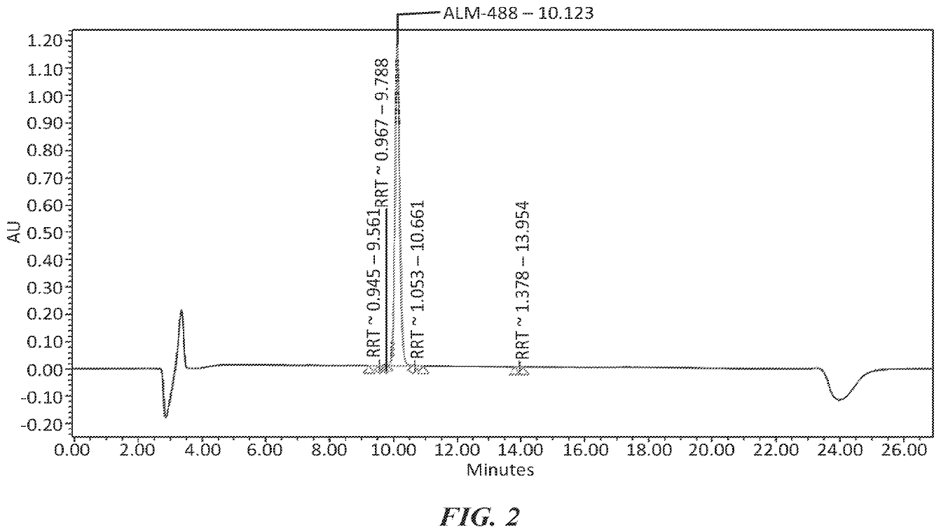

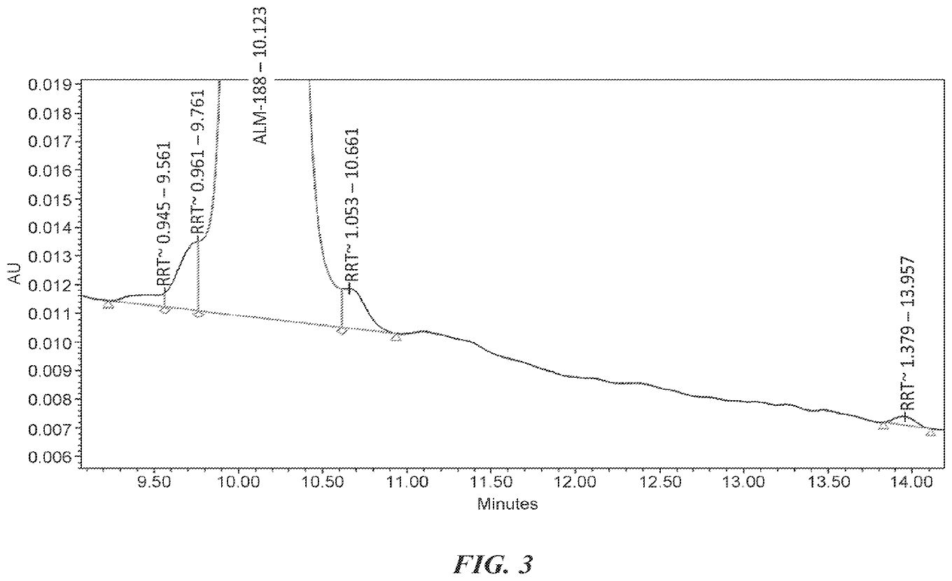

FIG. 2 illustrates an HPLC chromatogram for a 0.25 mg/mL solution of ALM-488 (as an HCl salt in water) that was analyzed using the HPLC conditions of Table 3. FIG. 3 shows a magnified view of the main peaks in the HPLC chromatogram of FIG. 2. Under the HPLC conditions of Table 3 (as illustrated in FIGS. 2 and 3), ALM-488 had a retention time of ~10 minutes with primary impurities eluting out with retention times of ~0.96 (0.15 area %) and ~1.06 (0.11 area %).

### Example 1: Preparation of Crude ALM-488

### A. Overview of Peptide Synthesis Process

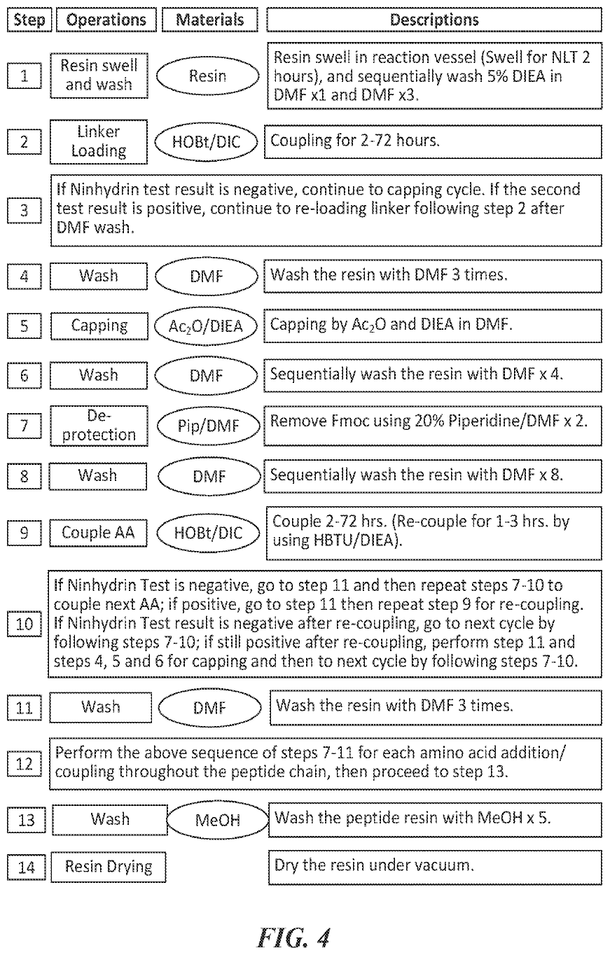

In the embodiment of Example 1, the ALM-488 peptide is assembled using a solid support MBHA resin starting at the C-terminal to the N-terminal of the peptide sequence. FIG. 4 provides a flow diagram of the solid support synthesis used to prepare ALM-488.

The Rink amide linker is coupled onto the MBHA resin, followed by Fmoc-Gly-OH. 5-FAM was then coupled onto the resulting resin. To couple the next Fmoc amino acid in the sequence, the Fmoc protecting group of the previous amino acid (from the peptide that is bound to the resin) is removed by de-protection using 20% piperidine/DMF. The resulting amino acid is then coupled to the N-terminal group of the previous amino acid. The manufacturing process employs two coupling methods. The primary coupling method is the DIC method for the initial coupling of all amino acids, and the secondary coupling method is the HBTU method for recoupling of the amino acids, if necessary.

### B. Resin Swell and Wash

Prior to coupling to the Rink amide linker, the starting resin is swelled and washed. The resin is then dispensed into a reaction vessel, and DMF is added to affect swelling (ratio of DMF to starting resin is 7 to 12 mL/g). The reaction is then stirred for no less than 2 hours and drained.

Additional DMF is then added into the vessel, followed by stirring and addition of 5% of DIEA. After 10 to 20 minutes of stirring, the reaction vessel is drained and additional DMF is added. The reaction vessel is then stirred for no less than 2 minutes and the solvent is then drained. The resin is washed two more times by repeating the washing step above. A ninhydrin test is performed to confirm the absence of residual amines and/or amino acids in the resulting resin.

### C. Fmoc-Rink Amide Linker Loading

The molar ratio of the Fmoc-Rink Amide Linker/HOBt (or HOBt-A)/DIC/synthesis scale was 1.5:1.5:1.5:1. The desired amounts of Fmoc-Rink Amide Linker and HOBt are dissolved in an amino acid (AA) mixing container with DMF. After the Fmoc-Rink Amide Linker/HOBt solution is fully dissolved, it is then transferred into a reaction vessel. The reaction vessel is then stirred and DIC is slowly added (allowable range for DIC was ±2.0 mL). Stirring of the reaction mixture then occurs for 2 to 72 hours, and a first ninhydrin test is performed at least two hours from the start time of the stirring. If the first ninhydrin test is negative, then the process is continued to the additional DMF washing step described below. If the first ninhydrin test is positive, then the reaction time is extended, and a second ninhydrin test is performed at least 1 hour after the first ninhydrin test. Continual ninhydrin testing is performed until the test is negative.

After a negative result from the ninhydrin test was obtained, the resin is washed three additional times with DMF and is stirred for at least 2 minutes after each of the washes. After each DMF wash, the solvent is drained from the reaction vessel. Additional DMF is then added to the resin and the resulting mixture is stirred. While stirring, 5% of DIEA and 5% of Ac₂O are added, and the reaction mixture is stirred for 15 to 25 minutes. The resin is then washed four times with DMF while being stirred for at least 2 minutes for each wash, and the solvent is drained from the reaction vessel after each wash.

### D. Interim De-Protection and Wash

The Fmoc protecting group is removed using a deprotection solution of 20% Pip/DMF. The deprotection solution is added to the reaction vessel containing the peptide resin, and the resulting suspension is stirred for 5 to 10 minutes. After stirring for this period, the solvent is drained from the reaction vessel and additional deprotection solution is added. The resulting suspension is stirred for an additional 20 to 30 minutes, and then the solvent is drained from the vessel. Using DMF, the resin is washed a total of 8 times and is stirred for at least 2 minutes for each wash. The solvent is drained from the reaction vessel after each of the 8 washes. For each AA cycle a ninhydrin test is performed, the process is stopped after a negative ninhydrin test is obtained.

### E. Amino Acid Coupling and Washing (DIC Method)

The molar ratio of Fmoc-amino acids/HOBt (or HOBt-A)/DIC/synthesis scale is 3:3:3:1, except for the AA cycle 1 of 5-Fam which uses a molar ratio of 1.5:1.5:1.5:1. The desired amount of Fmoc-AA-OH and HOBt is dissolved in an AA mixing container with DMF. Once the Fmoc-AA/HOBt solution was fully dissolved, it is transferred into a reaction vessel and is stirred for 2 to 72 hours while DIC is slowly added. A ninhydrin test was performed after at least 2 hours from the reaction start time. If a negative ninhydrin result is obtained, then the process may continue to the next cycle or final wash (only for cycle 1) after the DMF wash. If a positive ninhydrin test is obtained, then the reaction time is extended, and a second ninhydrin test is performed at least one hour after the first test. For DMF wash, the resin is washed a total of 3 times with DMF, is stirred for at least 2 minutes, and the solvent is then drained from the reaction vessel. When a negative result is obtained from the ninhydrin test, then the process is advanced to the next AA cycle. When a positive result is obtained from the ninhydrin test, a recoupling process of the amino acid is carried out.

### F. Amino Acid Recoupling and Washing

The HBTU/DIEA recoupling method is followed, after receiving a positive result from the ninhydrin test, as described above, during amino acid coupling. The desired amount of amino acid and reagent is dissolved in the AA mixing container with DMF (allowable range for AA, 5-FAM and HBTU are ±2.0 g). After the amino acid and reagent solution are fully dissolved, the resulting mixture is transferred to the reaction vessel and is stirred for 1 to 3 hours while DIEA is slowly added. A ninhydrin test is performed after at least 1 hour from the start time. If a positive result was obtained from the ninhydrin test, then the reaction time is extended, and an additional ninhydrin test is performed after at least 1 hour from the first test. When a negative result is obtained from the ninhydrin test, then the process may advance to the next cycle or to the final wash after the DMF wash. For the DMF wash, the resin is washed 3 times with DMF while being stirred for at least 2 minutes. The solvent is drained from the reaction vessel after each wash. If a negative result is obtained from the ninhydrin test, then the next AA cycle is continued by the same process. If continuous positive results from the ninhydrin test occur, then the capping process described below is carried out to deactivate the uncoupled amino group (except for AA cycle 1).

### G. Capping

Continuous positive results from the ninhydrin test during the re-coupling process described above is addressed by performing a capping process, in order to deactivate uncoupled amino groups. For this capping, DMF is added to the resin and the resulting mixture is stirred. While stirring, 5% of DIEA and 5% of Ac₂O are added and the mixture is stirred for a total of 15 to 25 minutes. After the stirring time is completed, the resin is washed 4 times with DMF and is stirred for at least 2 minutes during each wash.

### H. Peptide Manufacturing Synthesis and Cleavage Process

After addition of the final unit (i.e., the 5-FAM moiety), the resulting resin is washed 5 times with MeOH while being stirred for at least 2 minutes during each wash. For the final wash, the solvent is drained from the reaction vessel after each of the 5 washes. The peptide resin is washed with DMF followed by MeOH, and the resulting resin is dried under vacuum. Loss of weight on drying is used to gauge the completion of the drying process.

When the desired peptide resin is synthesized, the peptide is then separated from the solid support, and all the amino acid side chain protecting groups are removed to yield the desired peptide. In Fmoc SPPS, this step is carried out by treating the peptidyl resin with TFA containing scavengers which include EDT/TIS/water. The cleaved mixture is then precipitated, washed with ether, and dried to provide the ALM-488 crude peptide.

### Example 2: Purification of Crude ALM-488

The identity of the crude peptide (ALM-488) from Example 1 is verified using mass spectrometry (MS), and its purity is analyzed using analytical C18 reverse phase HPLC (RP-HPLC) under the HPLC method conditions summarized in Table 3 above.

### A. Separation by HPLC

The crude ALM-488 is dissolved with ACN and distilled water. A small quantity of acetic acid/ammonium hydroxide can be added, if necessary, to fully dissolve the crude ALM-488 into the ACN/water. The resulting homogeneous mixture is then purified using RP-HPLC with varying buffer systems. The buffer systems consist of an aqueous solution (buffer A) paired with an organic phase modifier consisting of 100% acetonitrile (buffer B).

The HPLC separation is carried out using the following steps:
1) Add purified water or aqueous buffer to dilute the peptide solution;
2) Equilibrate the RP-HPLC column with not less than two column volumes of aqueous buffer A and organic buffer B (acetonitrile);
3) Load the diluted peptide solution to the RP-HPLC column;
4) Equilibrate RP-HPLC column with not less than two column volumes of aqueous buffer A and organic buffer B (acetonitrile);
5) Run HPLC gradient, collecting eluent when there is an increase in absorption or at the corresponding % B for peptide elution; stop collecting when absorption drops to near its starting point; and
6) Wash HPLC column with a high percentage of organic buffer B and aqueous buffer A for not less than two column volumes.

During each purification step, the eluted fractions are collected, and their purities are analyzed by UPLC/HPLC. Fractions that satisfy the 1^{st} cut and 2^{nd} cut purity criteria are pooled, while fractions below the 2^{nd} cut purity criteria are discarded. The pooled 1^{st} cut fractions are advanced on to the next purification step, while the 2^{nd} cut fractions are combined for further HPLC purification.

### B. Ion Exchange of HPLC-Purified ALM-488

The combined 1^{st} cut fractions of the HPLC-purified ALM-488 are then ion exchanged to produce an ALM-488 chloride salt using the following procedure.

Purified water or aqueous buffer is added to dilute the HPLC-purified ALM-488. Then, the RP-HPLC column is equilibrated with at least three column volumes of an aqueous buffer A (1% triethylamine phosphate in water) and an organic buffer B (acetonitrile). Next, the diluted peptide solution is added to the RP-HPLC column.

The RP-HPLC column is then equilibrated with (i) at least three column volumes of aqueous buffer A (1% triethylamine phosphate in water) and organic buffer B (acetonitrile), (ii) followed by at least three column volumes of aqueous buffer A (50 mM ammonium acetate in water) and organic buffer B (acetonitrile), (iii) followed by at least three column volumes of aqueous buffer A (1% acetic acid in water) and organic buffer B (acetonitrile), (iv) followed by at least three column volumes of aqueous buffer A (0.01 N hydrochloric acid in water) and organic buffer B (acetonitrile).

Then HPLC gradient is performed, collecting eluent when there is an increase in absorption or at the corresponding % B for peptide elution, and collection is stopped when absorption drops to near its starting point. Finally, the HPLC column is washed with a high percentage of the organic buffer B and the aqueous buffer A for not less than two column volumes.

### C. Lyophilization of ALM-488 Chloride Salt

After subjecting the combined 1^{st} cut fractions to the ion exchange procedure described above, the combined 1^{st} cut is lyophilized using jar lyophilization. If multiple batches of lyophilization are needed to lyophilize the combined 1^{st} cut, then these separate lyophilizations are carried out for at least 12 hours, and they are followed by a final (combined) lyophilization for at least 72 hours. The separate lyophilized products may be stored at ≤−15° C. prior to performing the final (combined) lyophilization.

### Example 3: Analysis of Purified ALM-488

By the above procedure, the chloride salt of ALM-488 is provided as a yellow-orange solid. In other embodiments, the ion exchange process may be altered such that the ALM-488 product is in the form of a different salt, such as an acetate salt.

### A. HPLC Purity of Purified ALM-488

As illustrated in FIGS. 2 and 3, the chloride salt of ALM-488 eluted out with a retention time of ~10 min under the HPLC method of Table 3. Primary impurities were detected at relative retention times of ~0.96 and ~1.06 at levels of ~0.14 and ~0.11% area (see FIG. 3). Based on the HPLC analysis, the purity of the chloride salt of ALM-488 was 98.95%.

### B. Chloride Content of Purified ALM-488

The chloride content for the chloride salt of ALM488 was measured to be 3.57% w/w (3.67% w/w as HCl).

### C. Thermogravimetric Analysis of Purified ALM-488

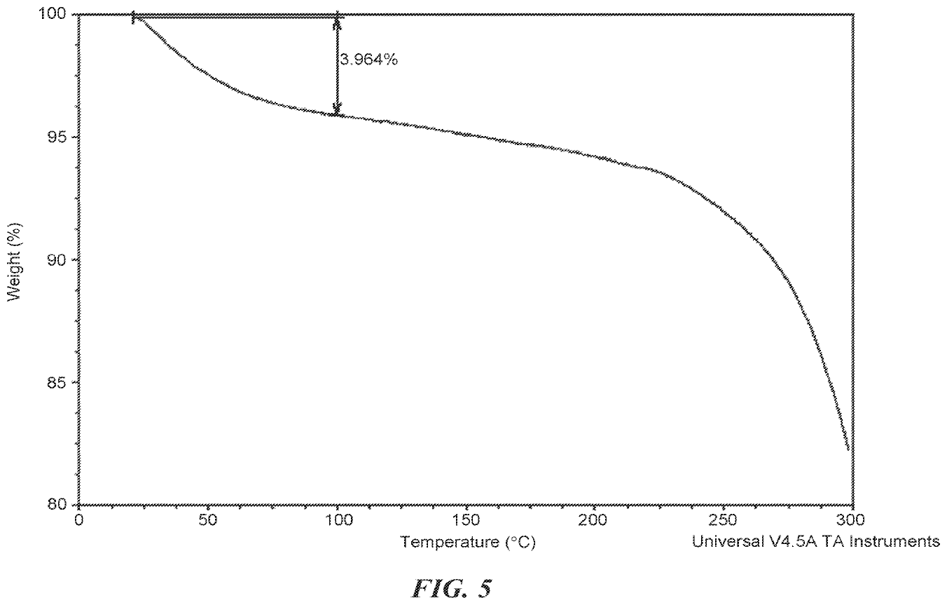

Thermogravimetric analysis (TGA) for the chloride salt of ALM-488 revealed a weight loss of 3.96% when the sample was heated to 100° C. using a Perkin Elmer TGA 7 thermogravimetric analyzer. FIG. 5 shows the thermogravimetric weight loss curve for the chloride salt of ALM-488. As shown in FIG. 5, further weight loss was gradual before a steep decrease in weight presumably due to degradation with an onset ~200-250° C.

### D. Water Content of Purified ALM-488

Karl Fisher titration was carried out on the chloride salt of ALM-488 using a Mettler Toledo Stromboli automatic oven sample changer. The instrument was standardized with a Hydranal® Water standard (Fluka). The solid ALM-488 sample was weighed into a sample vial and was heated to 100° C. in the oven, and the water vaporized from the sample was transferred to the titrator cell by purge gas. The measured water content in the sample was 4.08%, which was in agreement with the TGA results.

### E. Peptide Content of Purified ALM-488

Correcting for the measured contents of chloride and water in the chloride salt of ALM-488, the peptide content was calculated to be 91.28%. All concentrations and quantities of ALM-488 in the following disclosure are expressed in terms of the pure peptide free of both counter-ion and water. A correction multiplier factor of 1.096 was applied for the chloride salt of ALM-488, in order to determine the quantity of the free peptide for the purpose of weighing the ALM-488 for experiments and product manufacturing.

### F. Linear Relationship Between Amount of ALM-488 and HPLC Peak Area

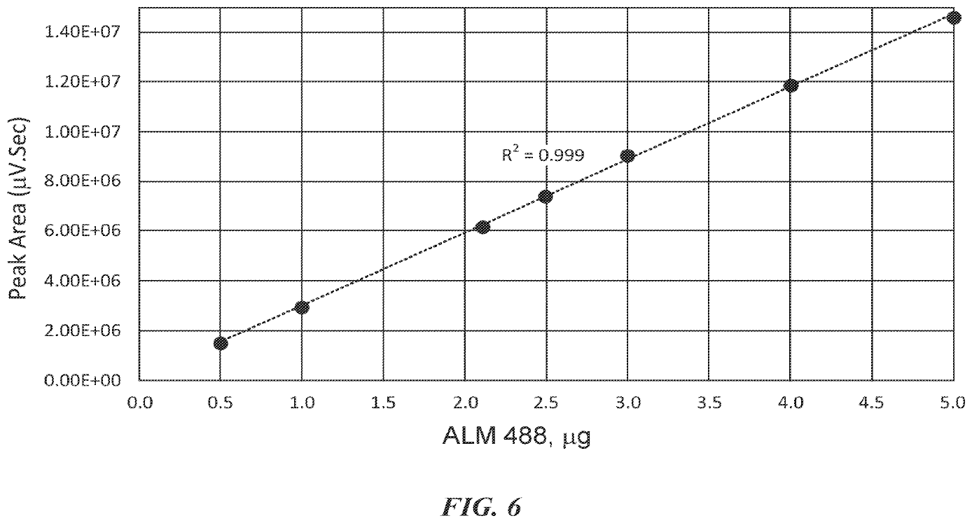

Standard solutions of ALM-488 at different concentrations and varying injection volumes were analyzed using the HPLC method described above. The linear relationship between the amount of injected ALM-488 and the area of the main chromatographic peak is shown in FIG. 6. At mass loadings beyond the range plotted in FIG. 6 (>5 μg of ALM-488), the peak areas increased less than proportionately with loading. All samples were suitably diluted to stay within the demonstrated linear range. Acceptable precision was obtained for repeat injections of the standard solution. For example, the relative standard deviation for the mean peak area from six 4 μL injections of an ~1 mg/mL ALM-488 solution in water was 0.35%.

### Example 4: Study of pH-Moderated Solubility of ALM-488 Salts

Studies were undertaken using both the chloride and acetate salts of purified ALM-488 to understand the relationship between pH and solubility.

### A. Solubility of HCl Salt of ALM-488 Based on pH

An initial visual assessment revealed a solubility of >180 mg/mL for the ALM-488, HCl salt with a solution pH of <pH 2.

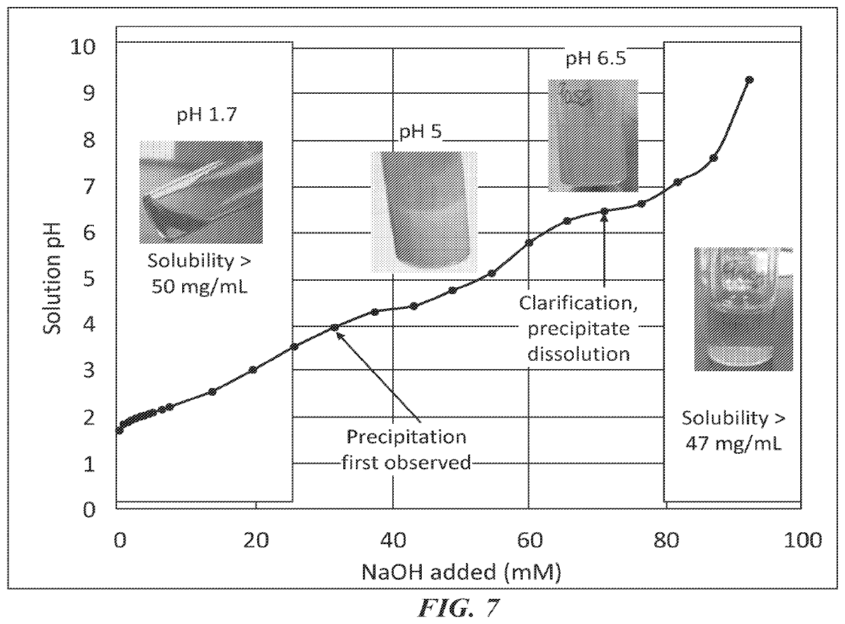

A titration experiment was carried out using the ALM-488, HCl salt to determine if a nominal 40-50 mg/mL or higher solubility could be retained as pH is increased by gradual addition of NaOH. A starting concentration of ~55 mg/mL (~24 mM) of the ALM-488, HCl salt in water (in terms of pure peptide) had a solution pH of 1.7. FIG. 7 shows a plot of the measured pH as a function of increasing concentration of added NaOH in the mixture. At solution pH values <4 the peptide remained in solution at concentrations >50 mg/mL. Above pH 4, the peptide precipitated. The extent of precipitation increased resulting in an opaque dispersion in the pH range of ~4.5 to 5.5. On further addition of NaOH, as the pH increased to ~6.5, peptide dissolution resulted in clarification of the mixture and complete dissolution was observed at pH values >7. Dilution of the mixture due to base addition decreased the peptide concentration to ~47 mg/mL at pH values >7. The shaded regions in FIG. 7 represent pH ranges where the ALM-488 peptide exhibits adequate solubility. As seen in the titration curve, resistance to pH change upon addition base was observed at pH values ~4.5 and ~6.5, which may coincide with the pKa values of glutamic acid and 5-FAM.

### B. Solubility of Chloride and Acetate Salts of ALM-488 Based on pH

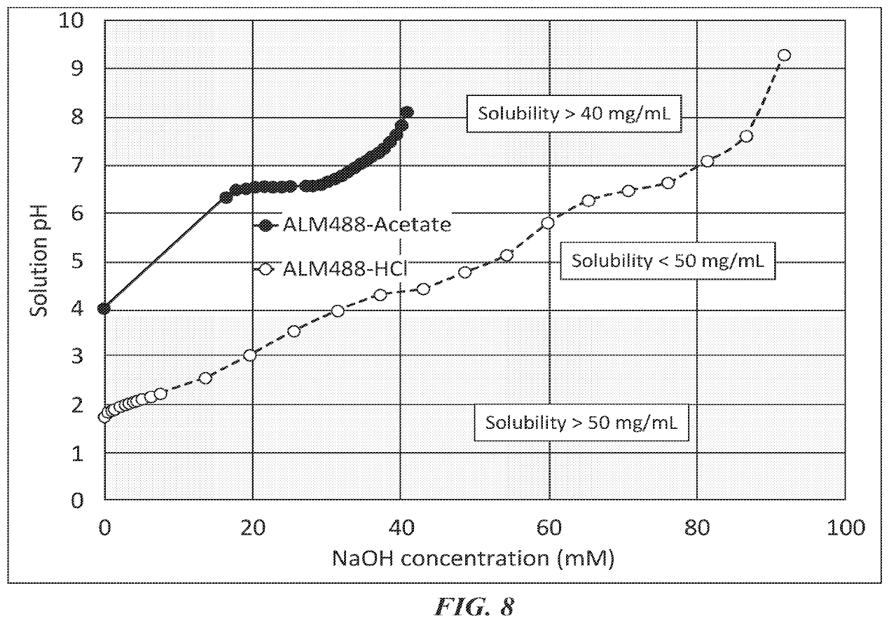

FIG. 8 is an overlay of the titration curves for ALM-488 hydrochloride and the ALM-488 acetate. As shown in FIG. 8, the pH of the initial ALM-488 acetate mixture (~50 mg/mL) was significantly higher (pH ~4) compared to the initial pH of the hydrochloride salt (pH ~1.8). At the initial pH ~4 the ALM-488 peptide of the acetate salt did not completely dissolve and formed an opaque orange dispersion. Increasing the pH of the acetate salt mixture by addition of NaOH resulted in precipitate dissolution and a clear solution at pH >7. The shaded regions in FIG. 8 (pH >7) represent pH ranges where the chloride and acetate salts of ALM-488 are expected to exhibit adequate solubility. As illustrated in FIG. 8, the amount of NaOH required to adjust the pH of an ALM-488 acetate solution to ~7 (in order to obtain adequate solubility and an acceptable pH for an intravenous formulation) was lower than that for the hydrochloride salt of ALM488.

### Example 5: Influence of pH on Stability of ALM-488

To understand pH-stability relationships, solutions of the ALM-488, HCl salt were prepared at ~1 mg/mL in 50 mM sodium citrate (pH 4-6) or sodium phosphate buffer (pH 7-8) solutions. As shown in Table 4 below, dissolution of ALM-488 was rapid and complete in water (pH 3.5) and phosphate buffer (pH 7-8). However, in citrate buffers (pH 4-6) the solubility of ALM-488 was <1 mg/mL, and incomplete dissolution led to excess undissolved solid. All mixtures were filtered through a 0.45 μm PVDF syringe filter to remove undissolved solid, prior to storage. The filtered solution pH values were measured, and the samples were analyzed by HPLC using the HPLC method conditions of Table 3. Table lists the measured concentrations of ALM-488 (after filtration) as well as the measured solution pH values.

TABLE 4
Measured ALM-488 concentrations in different
buffered vehicles and solution pH values

| | Measured AL488 | |
|---|---|---|
| | Concentration | Measured |
| | in filtered solution | ALM488 |
| Vehicle | (mg/mL) | solution pH |
| Water | 0.86 | 3.46 |
| Citrate buffer 50 mM | 0.83 | 3.85 |
| (pH 3.95) | | |
| Citrate buffer 50 mM | 0.18 | 4.89 |
| (pH 4.97) | | |
| Citrate buffer 50 mM | 0.61 | 5.91 |
| (pH 6.01) | | |
| Phos. buffer 50 mM | 0.94 | 6.94 |
| (pH 7.04) | | |
| Phos. buffer 50 mM | 0.95 | 7.90 |
| (pH 8.18) | | |

As shown in Table 4, the solubility of ALM-488 decreased significantly as the solution pH approached the isoelectric pH with a solubility of ~0.2 mg/mL at pH 5 (Citrate buffer 50 nM, pH=6.01). The solutions were then transferred into HPLC sample vials and were stored at 5° C., room temperature (22-23° C.) and 40° C.

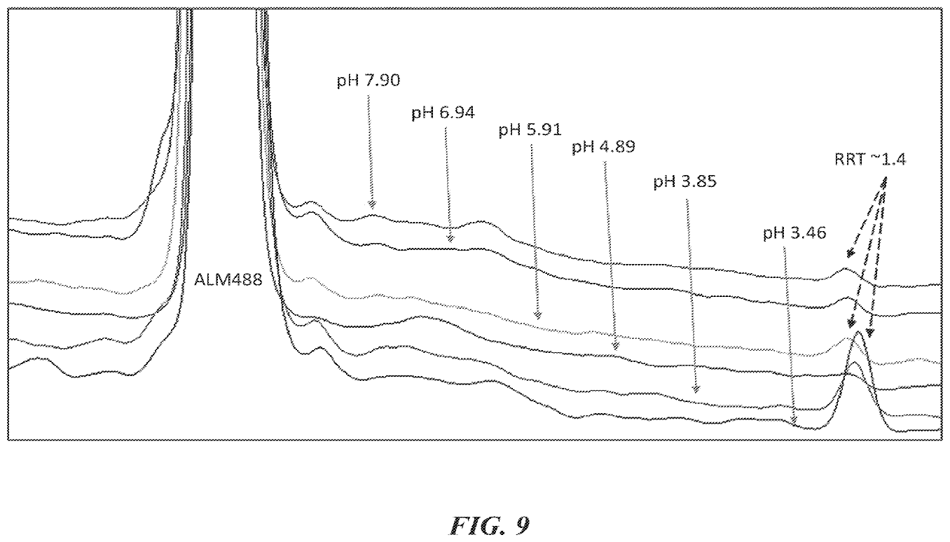

Table 5 below summarizes the measured concentrations of intact ALM-488 peptide quantified relative to a standard solution, as well as the area % of the main peptide peaks as measured using the HPLC analysis method of Table 3. FIG. 9 is an overlay of representative chromatograms of the ALM-488 solutions at different solution pH values stored at 40° C. for 24 h. As shown in this overlay, the impurity at RRT ~1.4 grew in solutions at lower pH values-most significantly in water (pH 3.46) to ~0.5 area %. At higher pH values, the impurity at RRT ~0.96 appeared to grow during storage.

TABLE 5
ALM-488 concentrations in solutions prepared at different pH
values, initially and after storate at 40° C. for 24 hours

| | ALM488 concentration | |
|---|---|---|
| | (HPLC purity, area % in parentheses) | |
| Vehicle | Initial | After 24 hours at 40° C. |
| Water | 0.86 mg/mL, (99.8%) | 0.86 mg/mL, (99.3%) |
| Citrate buffer 50 mM, pH 3.95 | 0.83 mg/mL, (99.3%) | 0.87 mg/mL, (99.5%) |
| Citrate buffer 50 mM, pH 4.97 | 0.18 mg/mL, (99.8%) | 0.18 mg/mL, (99.8%) |
| Citrate buffer 50 mM, pH 6.01 | 0.61 mg/mL | 0.60 mg/mL, (99.8%) |
| Phos. buffer 50 mM, pH 7.04 | 0.94 mg/mL, (99.7%) | 0.95 mg/mL, (99.8%) |
| Phos. buffer 50 mM, pH 8.18 | 0.95 mg/mL, (99.7%) | 0.96 mg/mL, (99.5%) |

The results of Example 5 show that ALM-488 has adequate solution stability at neutral pH values for further development as a freeze-dried product.

### Example 6: Influence of Storage Temperature on Stability of ALM-488

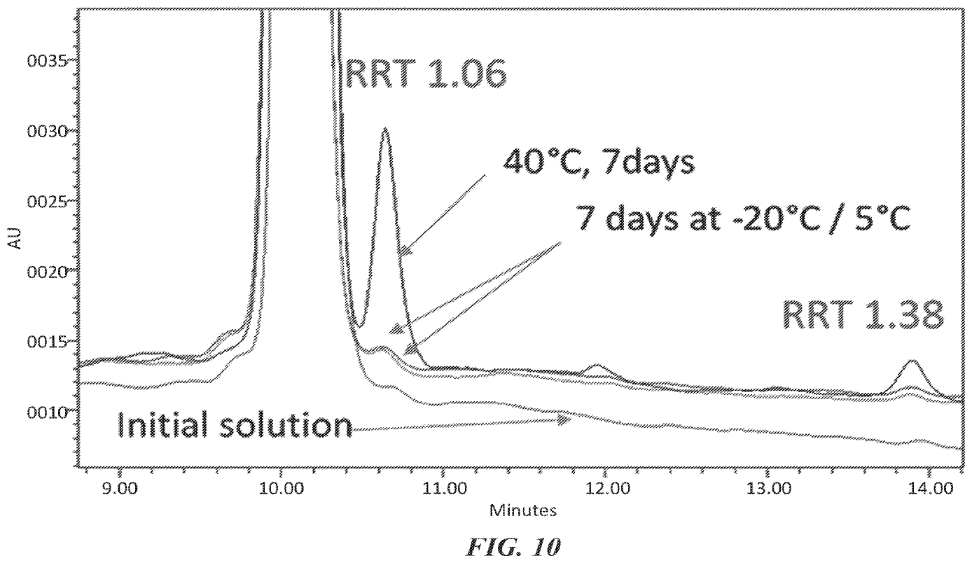

To evaluate whether the ALM-488, HCl salt can be supplied in the form of a concentrated frozen solution, 50 mg/mL solutions of the HCl salt were prepared in water and evaluated for storage stability at different temperatures. The solutions having pH of 1.87 were prepared in sterile water for injection, and these solutions were stored at different storage temperatures (−20° C., 5° C. and 40° C.) and were analyzed using the HPLC method of Table 3 at different time periods. Table 6 lists the chromatographic data for these sample solutions, and FIG. 10 shows an overlay of magnified chromatograms of ALM-488 solution (50 mg/mL) in water analyzed initially and after 7 days at different storage temperatures.

TABLE 6
Chromatographic data for ALM-488 50 mg/mL solution
in sterile water stored for 7 days at −20°
C., 5° C. and 40° C.

Condition

| | | | |
|---|---|---|---|
| Initial | 7 Days | 7 Days | 7 Days |
| (RT) | (−20° C.) | (5° C.) | (40° C.) |

| | |
|---|---|
| Component | AREA % |

| | | | | |
|---|---|---|---|---|
| RRT~0.73 | — | 0.07 | 0.07 | 0.05 |
| RRT~0.93 | — | — | — | 0.09 |
| RRT~0.94 | 0.09 | 0.03 | — | — |
| RRT~0.97 | 0.23 | 0.16 | 0.15 | 0.07 |
| ALM-488 | 99.64 | 99.56 | 99.61 | 97.96 |
| RRT~1.06 | 0.09 | 0.15 | 0.14 | 1.43 |
| RRT~1.11 | — | — | — | — |
| RRT~1.13 | — | — | — | — |
| RRT~1.16 | — | — | — | — |
| RRT~1.18 | — | — | — | 0.07 |
| RRT~1.38 | — | 0.04 | 0.05 | 0.27 |

A seen in Table 6 and in FIG. 10, the solution stored at 40° C. exhibited significant degradation resulting in the growth primarily of impurities at RRT ~1.06 and 1.38. The data also suggests that the levels of the impurity at RRT ~0.97 (which forms under higher pH conditions) were found to decrease in a temperature dependent fashion in the acidic solution. There was less degradation detected in the samples stored at reduced temperature (−20° C. and 5° C.).

### Example 7: Study of Organic Amine Compounds on Solubility of ALM-488 Salts

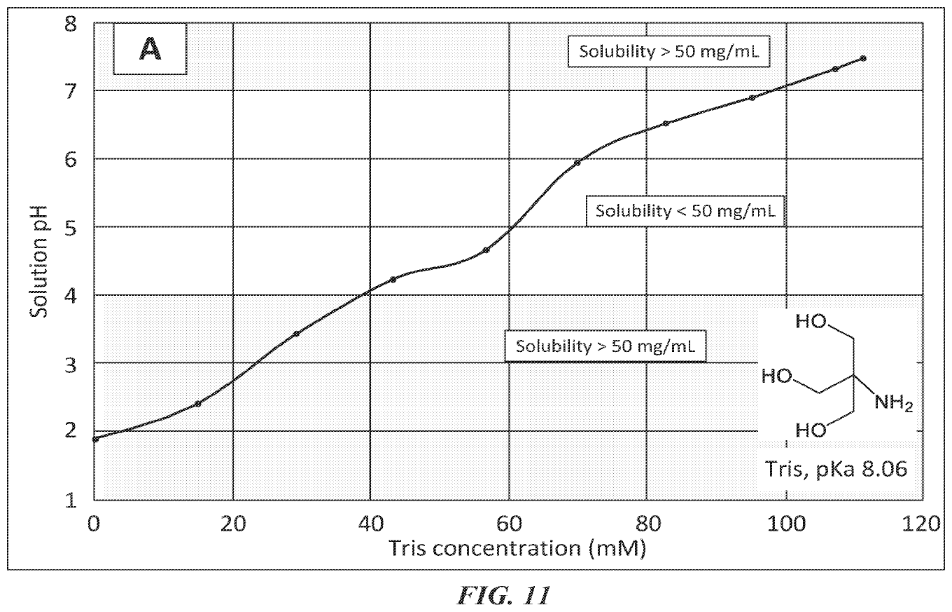

To assess formulations that could be used to prepare ~50 mg/vial samples of ALM-488, and samples having an ALM-488 concentration of ~50 mg/mL, formulations having different organic amine compounds were studied. Each of these samples included (i) ALM-488 hydrochloride, (ii) a suitable base appropriate for a freeze-dried formulation (to adjust solution pH to optimal 7-8, as described in Examples 4 and 5), and (iii) at least one organic amine compound functioning as a pH-adjusting agent, bulking agent and/or a tonicity-adjusting agent. Three organic amine compounds were tested: tromethamine (Tris, pKa 8.06), N-methyl glucamine (Meglumine, pKa 9.6) and L-arginine (pKa 12.5). 55-60 mg/mL solutions of ALM-488 (pH 1.8-1.9) were titrated by gradual addition of 750 mM arginine, 1 M Tris, or 1 M meglumine. The visual observations were similar to those in FIG. 7. As illustrated in FIGS. 11, 12 and 13, an increase in pH first caused precipitation of the ALM-488 peptide, followed by dissolution on continued base addition at pH >7. In each case the concentration of ALM-488 in the final solution at pH 7-8, was >50 mg/mL.

FIG. 11 is a pH titration curve showing pH as a function of added tromethamine (Tris) and how solubility of an HCl salt of ALM-488 is affected by pH. Calculated total ALM-488 peptide concentration in the sample ranged from 55 mg/mL (23.8 mM) with no Tris added (pH 1.89) to 49.2 mg/mL (21.3 mM) with 107 mm tris (pH 7.3). The shaded regions of FIG. 11 represent the pH ranges where a clear solution was obtained. Precipitation occurred near the iso-electric pH and solubility was <50 mg/mL in the pH range of ~4-7.

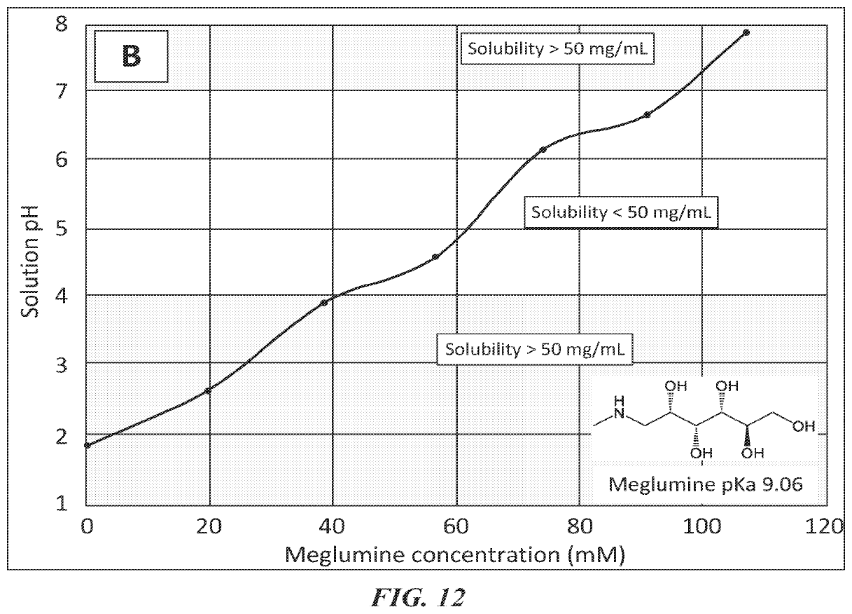

FIG. 12 is a pH titration curve showing pH as a function of added meglumine (N-methyl glucamine) and how solubility of an HCl salt of ALM-488 is affected by pH. Calculated total ALM-488 peptide concentration in the sample ranged from 55 mg/mL (23.8 mM) with no meglumine added (pH 1.86) to 49.2 mg/mL (21.3 mM) with 107 mm tris (pH 7.88). The shaded regions of FIG. 12 represent the pH ranges where a clear solution was obtained. Precipitation occurred near the iso-electric pH and solubility was <50 mg/mL in the pH range of ~4-7.

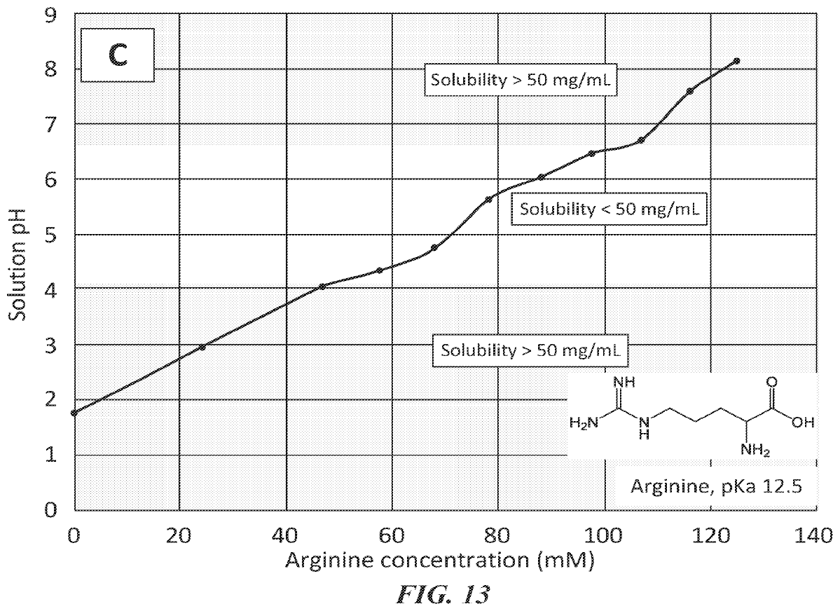

FIG. 13 is a pH titration curve showing pH as a function of added L-arginine (N-methyl glucamine) and how solubility of an HCl salt of ALM-488 is affected by pH. Calculated total ALM-488 peptide concentration in the sample ranged from 55 mg/mL (23.8 mM) with no L-arginine added (pH 1.86) to 51.7 mg/mL (22.4 mM) with 116 mM arginine (pH 7.61). The shaded regions of FIG. 13 represent the pH ranges where a clear solution was obtained. Precipitation occurred near the iso-electric pH and solubility was <50 mg/mL in the pH range of ~4-6.6.

As seen from the titration curves of FIGS. 11-13, organic base concentrations of ~110 mM yielded a near neutral solution pH and adequate drug solubility.

The bases that were investigated have been used on marketed drug products. Arginine is included in several approved IV products in substantially higher quantities than anticipated for ALM-488 formulations based on the study of Example 7. For example, the arginine dose from the max dose of Azactam® injection is 6.2 g per day, and from the max dose of Caldolor® injection is 3.2 g/day. Tromethamine, if used as the base with ALM-488 would require quantities exceeding the typical amounts encountered when tromethamine is used as a buffer. However, the maximum daily dose of Ketorolac tromethamine by IV injection (120 mg) is equivalent to ~38 mg of tromethamine. Tham® (Tromethamine injection) is a 300 mM (3.6%) solution of tromethamine used in metabolic acidosis and administered as slow infusion in much higher doses. A total daily dose of Baxdela® (delafloxacin meglumine injection, 300 mg twice daily) is equivalent to a meglumine dose of 384 mg.

The anticipated doses when using tromethamine (Tris), meglumine (N-methyl glucamine) and L-arginine corresponding to prepare 50 mg of ALM-488 formulations are expected to be much lower than the quantities of these bases contained in the approved drug products described above.

### Example 8: ALM-488 Compositions for Stability Studies

A series of sample formulations of ALM-488 were prepared in the pH range of 7-8 using different organic amine additives and combinations thereof. As summarized in Table 7, formulations using L-arginine, meglumine, glycine, mannitol and trehalose dihydrate, and combinations thereof, were prepared. The formulations were designed to obtain 25 mg/mL solutions of ALM-488 with enough of the respective base(s) to adjust solution pH to 7-8. With a 2 mL solution fill per vial, a concentration of 50 mg/vial of the ALM-488 peptide would be achieved. Lyophiles of the ALM-488 formulations are expected to be reconstituted to solutions having a volume of 1 mL, thus obtaining 50 mg/mL solutions of the ALM-488 peptide. The sample formulations of Table 7 were designed to target an osmolality of ~300 mOsm/kg for the finally reconstituted 50 mg/mL solutions.

TABLE 7
ALM-488 Compositions Prepared for Assessing Solution Stability

| | Samples | | | | | | |
|---|---|---|---|---|---|---|---|
| | 8-1 | 8-2 | 8-3 | 8-4 | 8-5 | 8-6 | 8-7 |
| Ingredient | Quantity (mg/mL) | | | | | | |
| ALM-488 | 27.4 | 27.4 | 27.4 | 27.4 | 27.4 | 27.4 | 27.4 |
| (25.0 mg peptide per mL) | | | | | | | |
| L-Arginine, USP | 9.4 | | 9.4 | 9.4 | | | 9.4 |
| Meglumine, USP | | 9.8 | | | 9.8 | 9.8 | |
| Glycine, USP | | | 5.6 | | 5.3 | | |
| Mannitol, USP | | | | 13.6 | | 12.7 | |
| Trehalose dihydrate USP | | | | | | | 28.2 |
| Sterile Water for Injection | q.s. to 1 mL | | | | | | |
| Solution pH | 7.53 | 7.52 | 7.53 | 7.66 | 7.44 | 7.43 | 7.44 |
| Osmolality (mOsm/kg) | 71 | 76 | 148 | 151 | 147 | 150 | ND |

As seen in Table 7, all of the sample formulations had a pH of ~7.5. Samples 8-1 and 8-2, which only included a single pH adjusting agent (L-arginine and meglumin respectively), had solution osmolalities of ~70 mOsm/kg. However, the use of combinations of pH adjusting agents in the Samples 8-3 thru 8-7 resulted in significantly higher solution osmolalities (148-151 mOsm/kg). The concentrations of the pH adjusting agents, when used in combination, were calculated to yield osmolality values of 140-150 mOsm/kg in the pre-lyophilization solution (25 mg/mL of ALM-488). When resulting lyophiles are reconstituted with water to a 50 mg/mL ALM-488 solution, the osmolality is expected to be in the desirable range of ~280-300 mOsm/kg.

The sample solutions were filtered through a 0.22-micron PVDF syringe filter, and aliquots of these filtered solutions were filled into USP Type 1 amber glass vials, stoppered with lyophilization rubber stoppers, and crimp sealed. The resulting sample vials were stored at 5° C. and 40° C. conditions. Then, after 7 and 14 days of storage, the sample solutions were diluted 100× with water and were analyzed by HPLC method conditions of Table 3.

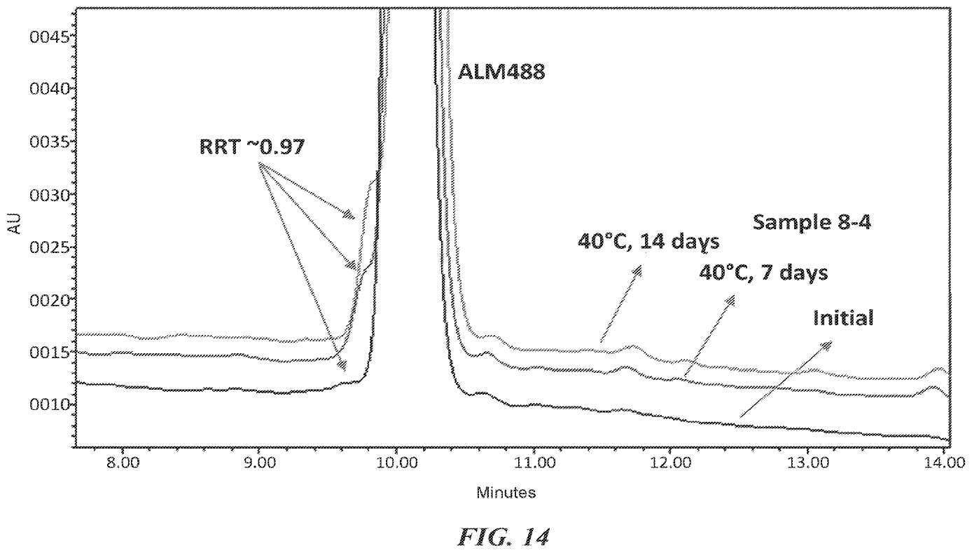

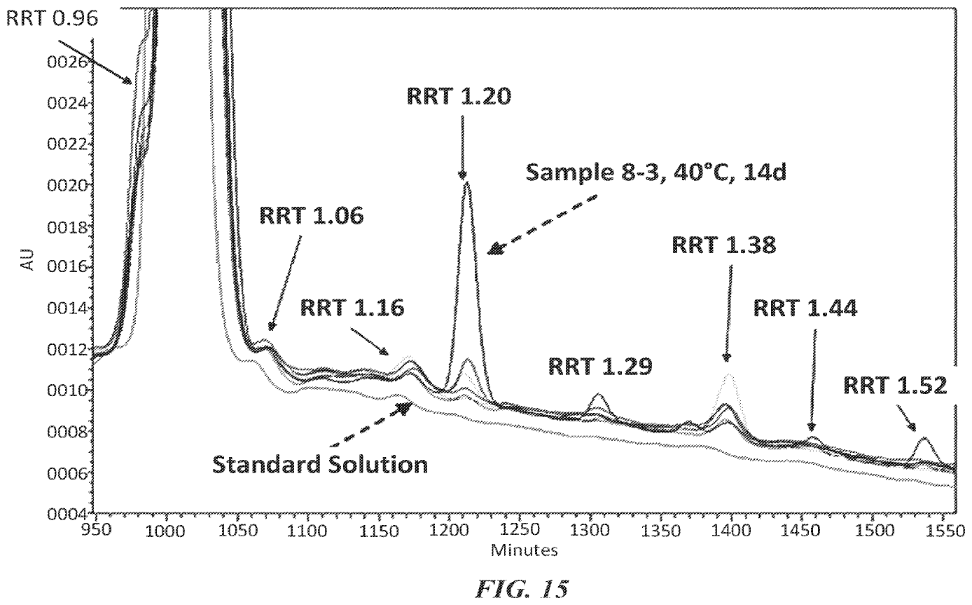

Tables 8-11 summarize the chromatographic data for the Samples 8-1 thru 8-7. FIG. 14 is an overlay of magnified HPLC chromatograms for the Sample 8-4 at initial preparation, after storage at 40° C. for 7 days, and after storage at 40° C. for 14 days. FIG. 15 is an overlay of magnified HPLC chromatographs for Samples 8-1 thru 8-7 of Example 8 after storage at 40° C. for 14 days.

TABLE 8
Chromatographic Data for Samples 8-1 and 8-3 before and after storage
at 5° C. or 40° C. for 7 days or 14 days

| | Sample | | | | | | | | | |
|---|---|---|---|---|---|---|---|---|---|---|
| | 8-1 | | | | | 8-3 | | | | |
| | (Arginine) | | | | | (Arginine-Glycine) | | | | |
| | Condition | | | | | | | | | |
| | | 5° C. | 5° C. | 40° C. | 40° C. | | 5° C. | 5° C. | 40° C. | 40° C. |
| | Initial | 7 d | 14 d | 7 d | 14 d | Initial | 7 d | 14 d | 7 d | 14 d |
| Component | Area % | | | | | Area % | | | | |
| RRT~0.73 | 0.06 | 0.05 | — | 0.05 | — | 0.07 | 0.05 | — | 0.08 | — |
| RRT~0.97 | 0.16 | 0.13 | 0.17 | 0.44 | 0.73 | 0.09 | 0.11 | 0.18 | 0.45 | 0.87 |
| ALM-488 | 99.58 | 99.59 | 99.68 | 98.99 | 98.75 | 99.68 | 99.54 | 99.50 | 98.33 | 97.48 |
| RRT~1.06 | 0.13 | 0.12 | 0.10 | 0.13 | 0.11 | 0.13 | 0.11 | 0.12 | 0.11 | 0.13 |
| RRT~1.16 | 0.07 | 0.07 | 0.06 | 0.12 | 0.12 | — | 0.05 | 0.06 | 0.11 | 0.17 |
| RRT~1.20 | — | — | — | 0.08 | 0.16 | — | 0.09 | 0.16 | 0.68 | 0.88 |
| RRT~1.29 | — | — | — | — | — | — | — | — | 0.12 | 0.13 |
| RRT~1.38 | — | 0.08 | — | 0.17 | 0.15 | — | 0.06 | — | 0.09 | 0.11 |
| RRT~1.44 | — | — | — | — | — | — | — | — | — | 0.08 |
| RRT~1.52 | — | — | — | — | — | — | — | — | — | 0.12 |

TABLE 9
Chromatographic Data for Samples 8-4 and 8-7 before and after storage
at 5° C. or 40° C. for 7 days or 14 days

| | Sample | | | | | | | | | |
|---|---|---|---|---|---|---|---|---|---|---|
| | 8-4 | | | | | 8-7 | | | | |
| | (Arginine - Mannitol) | | | | | (Arginine-Trehalose) | | | | |
| | Condition | | | | | | | | | |
| | | 5° C. | 5° C. | 40° C. | 40° C. | | 5° C. | 5° C. | 40° C. | 40° C. |
| | Initial | 7 d | 14 d | 7 d | 14 d | Initial | 7 d | 14 d | 7 d | 14 d |
| Component | Area % | | | | | Area % | | | | |
| RRT~0.73 | 0.05 | 0.04 | — | 0.07 | — | | 0.05 | 0.04 | 0.05 | 0.04 |
| RRT~0.97 | 0.11 | 0.10 | 0.15 | 0.59 | 1.00 | 0.12 | 0.12 | 0.11 | 0.31 | 0.60 |
| ALM-488 | 99.68 | 99.58 | 99.69 | 98.96 | 98.49 | 99.54 | 99.45 | 99.50 | 99.25 | 98.97 |
| RRT~1.06 | 0.12 | 0.11 | 0.10 | 0.12 | 0.11 | 0.10 | 0.12 | 0.11 | 0.12 | 0.10 |
| RRT~1.16 | — | 0.07 | 0.07 | 0.07 | 0.17 | — | 0.13 | 0.13 | 0.11 | 0.14 |
| RRT~1.20 | — | — | — | — | 0.03 | — | — | | | |
| RRT~1.29 | — | — | — | — | 0.04 | — | | | | |
| RRT~1.38 | — | 0.09 | — | 0.16 | 0.20 | — | 0.02 | 0.02 | 0.08 | 0.09 |
| RRT~1.44 | — | — | — | — | — | — | | | | |
| RRT~1.52 | — | — | — | — | — | — | | | | |

TABLE 10
Chromatographic Data for Samples 8-2 and 8-5 before and after storage
at 5° C. or 40° C. for 7 days or 14 days

| | Sample | | | | | | | | | |
|---|---|---|---|---|---|---|---|---|---|---|
| | 8-2 | | | | | 8-5 | | | | |
| | (Meglumine) | | | | | (Meglumine-Glycine) | | | | |
| | Condition | | | | | | | | | |
| | | 5° C. | 5° C. | 40° C. | 40° C. | | 5° C. | 5° C. | 40° C. | 40° C. |
| | Initial | 7 d | 14 d | 7 d | 14 d | Initial | 7 d | 14 d | 7 d | 14 d |
| Component | Area % | | | | | Area % | | | | |
| RRT~0.73 | 0.05 | 0.05 | — | 0.06 | — | 0.05 | 0.05 | — | 0.06 | — |
| RRT~0.97 | 0.11 | 0.12 | 0.18 | 0.42 | 0.75 | 0.10 | 0.12 | 0.20 | 0.39 | 0.63 |
| ALM-488 | 99.71 | 99.61 | 99.26 | 98.95 | 98.63 | 99.67 | 99.60 | 99.17 | 99.12 | 98.63 |
| RRT~1.06 | 0.10 | 0.12 | 0.17 | 0.13 | 0.13 | 0.13 | 0.12 | 0.19 | 0.12 | 0.17 |
| RRT~1.10 | — | — | — | — | — | — | — | 0.09 | — | 0.07 |
| RRT~1.13 | — | — | — | — | — | — | — | 0.12 | — | 0.11 |
| RRT~1.16 | — | 0.07 | 0.38 | 0.20 | 0.12 | — | 0.07 | 0.20 | 0.15 | 0.21 |
| RRT~1.20 | — | — | — | — | — | — | — | 0.03 | 0.04 | 0.05 |
| RRT~1.38 | — | 0.05 | — | 0.24 | 0.38 | — | 0.06 | — | 0.15 | 0.12 |

TABLE 11
Chromatographic Data for Samples 8-6 before and after storage
at 5° C. or 40° C. for 7 days or 14 days

| | Sample | | | | |
|---|---|---|---|---|---|
| | 8-6 | | | | |
| | (Meglumine-Mannitol) | | | | |
| | Condition | | | | |
| | | 5° C., | 5° C., | 40° C., | 40° C., |
| | Initial | 7 d | 14 d | 7 d | 14 d |
| Component | Area % | | | | |
| RRT~0.73 | 0.04 | 0.05 | — | 0.04 | — |
| RRT~0.97 | 0.10 | 0.10 | 0.15 | 0.36 | 0.64 |
| ALM-488 | 99.70 | 99.64 | 99.42 | 99.07 | 98.84 |
| RRT~1.06 | 0.12 | 0.12 | 0.17 | 0.14 | 0.11 |
| RRT~1.10 | — | — | 0.07 | — | — |
| RRT~1.13 | — | — | 0.09 | — | — |
| RRT~1.16 | — | 0.06 | 0.12 | 0.17 | 0.13 |
| RRT~1.20 | — | — | — | 0.07 | 0.09 |
| RRT~1.38 | — | 0.05 | — | 0.16 | 0.19 |

As shown in Tables 8-11, the primary common impurity occurred at RRT ~0.97. The impurity at RRT ~1.20 appeared to be more prevalent in the Sample 8-3 containing arginine and glycine, see FIG. 15. With the exception of the arginine-glycine solution (Sample 8-3), which appeared to lose ~2% of intact ALM-488, all the sample formulation lost only ~1% of intact ALM-488 expressed as area % of the main peak after 14 days at 40° C.

### Example 9: ALM-488 Compositions for Stability Studies

The sample formulations 8-1 through 8-7 were subjected to sub-ambient DSC experiments. Solution samples (~25 μL) were transferred to a DSC pan and were hermetically sealed before subjecting to cool-heat cycles. In a typical run, a sample is cooled to −50° C. and held isothermal to ensure maximal freezing and formation of a freeze-concentrated phase. This is followed by heating the sample at 2° C. per minute to a temperature beyond the ice melting event. A nitrogen gas purge of 40 mL/min is maintained in the DSC chamber. While sharp ice-crystallization exotherms were observed during cooling of the sample formulations, no solute crystallization events were noted.

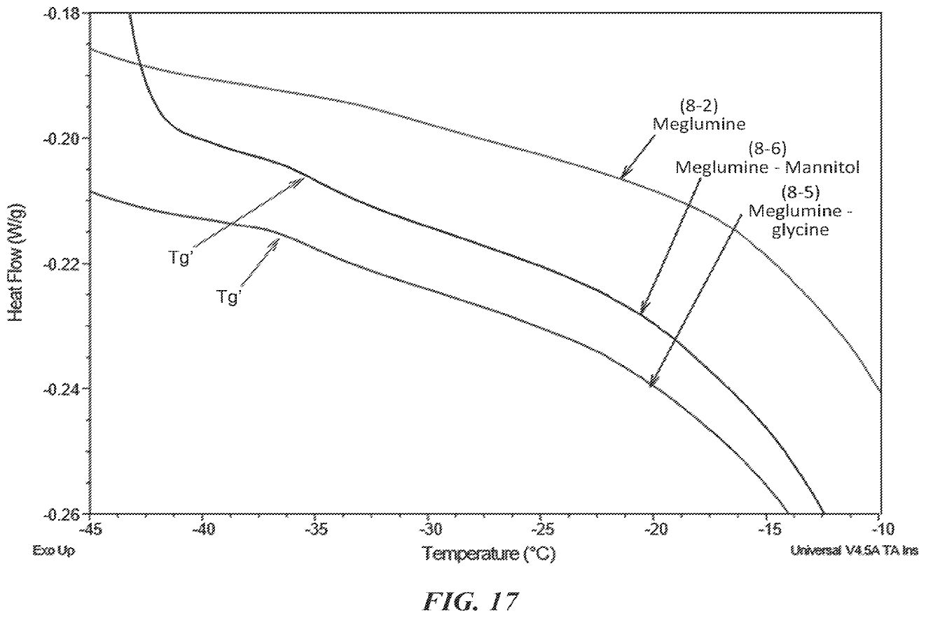

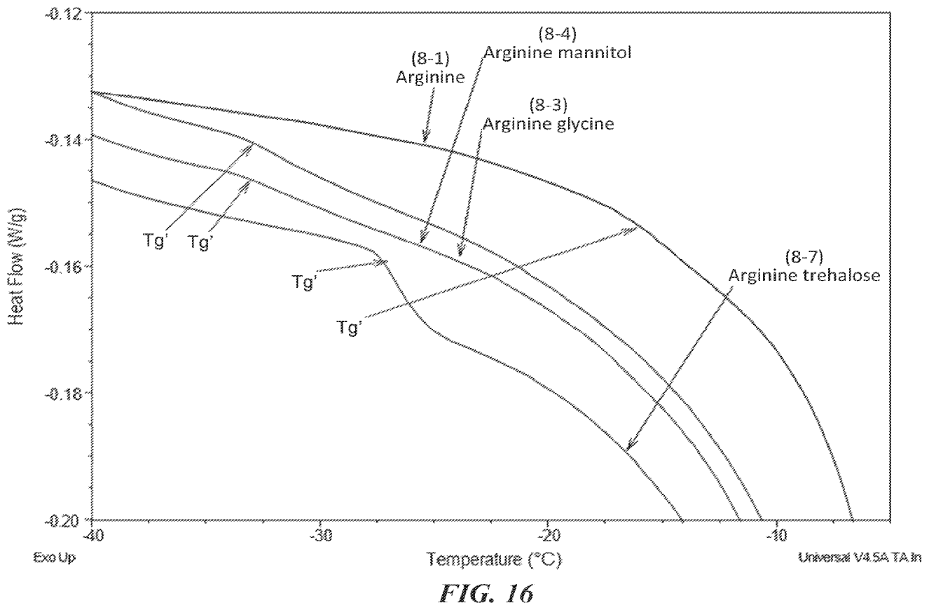

The heating curves of the frozen solutions for the arginine-containing formulations (Samples 8-1, 8-3, 8-4 and 8-7) are overlaid in FIG. 16, and the heating curves of the frozen solutions for the meglumine-containing formulations (Samples 8-2, 8-5 and 8-6) are overlaid in FIG. 17.

As illustrated in FIG. 16, the ALM488-arginine frozen formulation (8-1) exhibited a weak glass transition (Tg′) with an onset of about −16° C. When either mannitol (8-4) or glycine (8-3) were included as additional solutes, their crystallization appeared to be inhibited. Retention of mannitol (8-4) or glycine (8-3) in the freeze-concentrated amorphous phase resulted in a decrease in the Tg′, with an onset of about −33° C. and about −34° C. respectively. The arginine-trehalose formulation (8-7) was also retained amorphous during solution freezing, and also resulted in a decrease in the Tg′ (onset of around −28° C.).

As illustrated in FIG. 17, An ALM488-meglumine frozen formulation (8-2) did not reveal a strong glass transition temperature. The crystallization of the meglumine-mannitol formulation (8-6) and the meglumine-glycine formulation (8-5) during freezing appeared to be inhibited as well. This resulted in a single broad glass transition with an onset temperature of around −36° C. and around −37° C. respectively.

Thermal treatment of the frozen solutions at −15° C. in the DSC increased the Tg′ onset by ~2° C., suggesting that the crystallization is substantially inhibited by the co-solutes, ALM488 and arginine/meglumine.

Table 12 below summarizes DSC and FDM (freeze-drying microscopy) data for Samples 8-1 through 8-7.

Lyostat 2 is a fully integrated freeze-drying microscope that enables determination of critical parameters of formulations to be freeze-dried. Using this method, a liquid film of 2 μL of the sample solution was placed between a quartz slide and a glass cover slip, and is then sealed within the cryo-stage of the freeze drying microscope. In a typical experimental, the sample is frozen with the aid of liquid nitrogen to about −50° C. and is then held for 30 minutes to ensure maximal freezing of the solution. When a frozen film is obtained, ice sublimation is initiated by reducing the chamber pressure to about 150 mTorr. This results in drying of the film from the periphery towards the center. When a freeze-dried peripheral region can be visualized in the image, the sample stage is heated at an appropriate rate, and images are captured at regular intervals to visualize thermal events.

The first visual evidence of a collapse event (due to increased mobility of the freeze-concentrate) is listed in Table 12 along with the glass transition onset temperatures, for each of the Samples 8-1 through 8-7. In each case, the visual observation of a collapse event was observed at a temperature much higher than the onset of the broad glass transition event.

TABLE 12
Glass transition onset temperatures measured by DSC and collapse
temperatures measured by FDM for 25 mg/mL ALM-488 solutions

| | Onset of step change | First evidence of collapse |
|---|---|---|
| Formula (mg/g) | observed by DSC | observed by FDM |
| (8-1) | −16° C. | −10° C. |
| Arginine | | |
| (8-2) | Not detected | −15° C. |
| Meglumine | | |
| (8-3) | −34° C. | −26° C. |
| Arginine-Glycine | | |
| (8-4) | −33° C. | −23° C. |
| Arginine - Mannitol | | |
| (8-5) | −37° C. | −26° C. |
| Meglumine- Glycine | | |
| (8-6) | −36° C. | −25° C. |
| Meglumine-Mannitol | | |
| (8-7) | −28° C. | Not determined |
| Arginine - trehalose | | |

The arginine sample formulation (8-1) was chosen to be used in the lyophilization studies described below, given its use in several approved parenteral products, and the slightly higher glass transition and collapse temperatures observed with arginine-containing formulation (8-1) compared to the other sample formulations. The arginine-glycine formulation (8-3) was not chosen for the further studies due to its slightly inferior solution stability compared to other solutions.

### Example 10: Initial Lyophilization Studies

The sample ALM-488 formulations containing arginine (10-1), arginine-mannitol (10-2) and arginine-trehalose (10-3), as summarized in Table 13 below, were prepared in amber glass vials for initial lyophilization studies. In all three of these samples, the molar ratio of ALM-488 peptide to HCl was about 1:2.5 (peptide/HCl), and the molar ratio of ALM-488 peptide to arginine was about 1:5 (peptide/arginine). Each sample was prepared in a 5 mL amber glass vial (20 mm opening, USP Type I tubing glass), with 20 mm lyophilization stoppers (FluoroTech V10-F597 4432/50, West Pharmaceutical Services), and with a 20 mm flip-off aluminum crimp seal.

TABLE 13
ALM-488 Compositions for Initial Lyophilization Studies

| | 10-1 | | 10-2 | | 10-3 | |
|---|---|---|---|---|---|---|
| Ingredient | (Qty per vial) | | (Qty per vial) | | (Qty per vial) | |
| ALM-488 (HCl) ¹ | 50 | mg | 50 | mg | 50 | mg |
| L-Arginine USP | 18.8 | mg | 18.8 | mg | 18.8 | mg |
| Mannitol USP | — | | 27.2 | mg | — | |
| Trehalose dihydrate USP | — | | — | | 56.4 | mg |
| Sterile Water for Injection | qs to 2.0 g | | qs to 2.0 g | | qs to 2.02 g | |
| (qs to 2 mL/vial) ² | | | | | | |
| Pre-lyophilization solution density | 1.00 | g/cc | 1.00 | g/cc | 1.01 | g/cc |
| Batch Size | 50 | vials | 50 | vials | 50 | vials |

| | | | |
|---|---|---|---|
| Solution pH | 7.65 | 7.64 | 7.44 |

¹ Chloride content (3.57% w/w; 3.67% w/w as HCl) and water content−4.08%, by KFT, peptide purity = 98.95%; 50 mg of ALM-488 = (50 × 100)/(0.9895) × (100 −4.08 −3.67) = 54.78 mg of drug substance.
² The density of the pre-lyophilization compositions was determined to be 1.00 g/cc (Sample 10-1), 1.00 g/cc (Sample 10-2) and 1.01 g/cc (Sample 10-3). Using this density, the quantity of total solution fill per vial was calculated as listed in table. Water is removed during processing.

### A. Solution Compounding and Vial Filling

For each sample, the ALM-488 peptide it was first weighed out into a suitable container for preparation of the solution. Approximately 80% of the estimated batch quantity of water is then added slowly to the peptide, and the mixture is stirred to dissolve the drug. The batch quantity of arginine is then added to the peptide solution while mixing to create a vortex. Addition of arginine initially causes drug precipitation, but a clear orange solution is obtained on mixing. For compositions containing mannitol or trehalose, the additional agent is then added to the solution and stirred to yield a clear solution. The total solution weight is then made up with sterile water for injection. The resulting solution is then filtered through a 0.2-micron PVDF filter unit, and the filtered solution is sealed within a vial for lyophilization fill weight tolerance of #2%.

### B. Lyophilizations

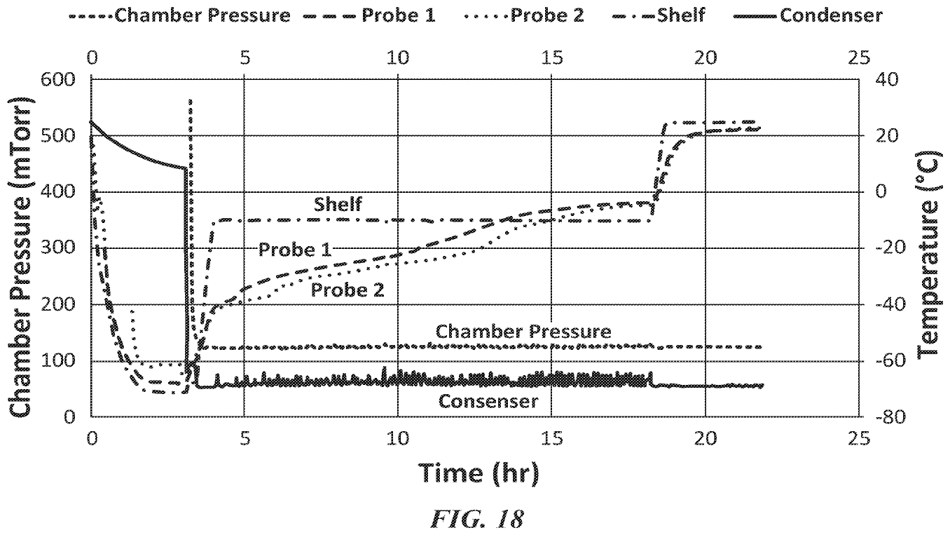

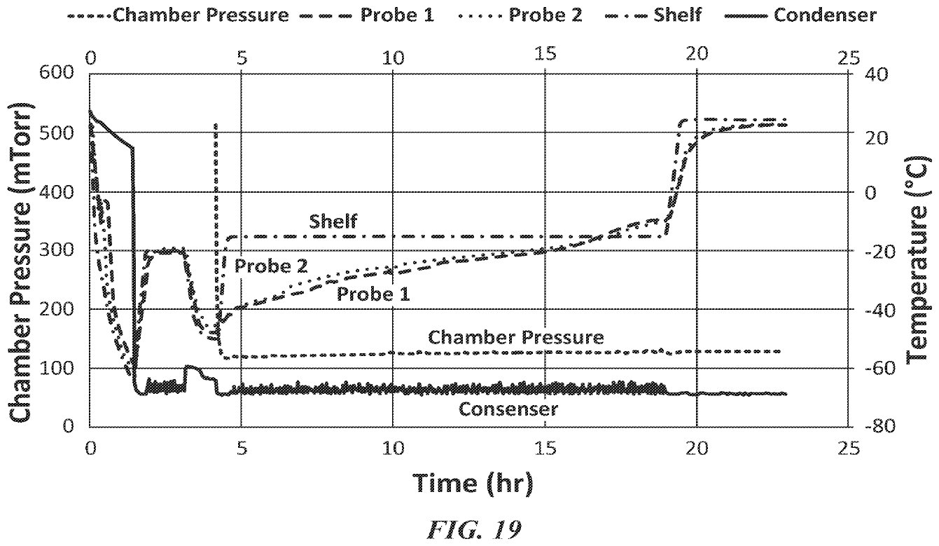

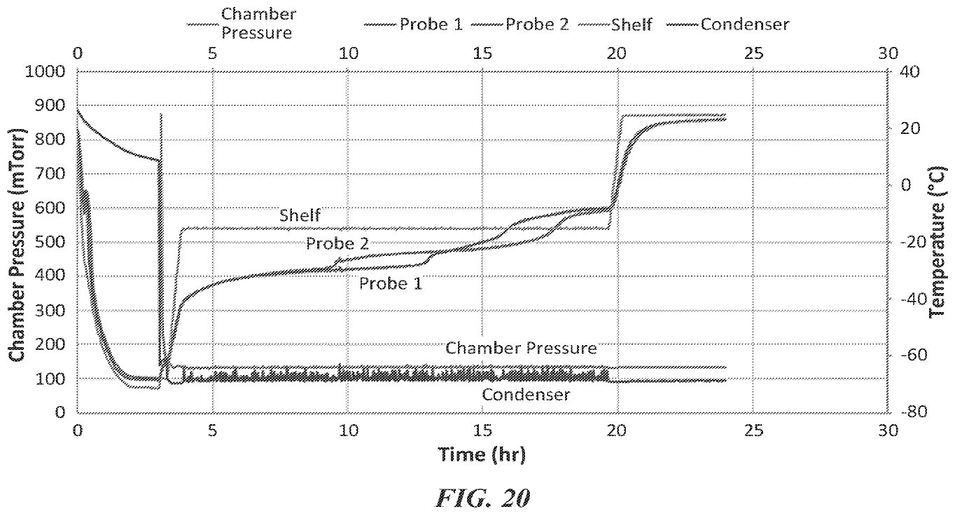

The processing parameters for the three compositions is summarized in Table 14 and the corresponding freeze-drying process graphs are shown in FIGS. 18 to 20.

TABLE 14
Comparison of Processing Parameters for Trial Lyophilizations

| Process | 10-1 | 10-2 | 10-3 |
|---|---|---|---|
| step | (Arginine) | (Arginine-Mannitol) | (Arginine-Trehalose) |
| Freezing | Solutions were placed on the freeze-dryer shelf at room temperature. The shelf was cooled | | |
| | to freeze the solution samples. Sample temperatures were about −60° C. to about −70° C. the | | |
| | end of freezing. | | |
| Thermal | No thermal treatment | Frozen solutions were annealed | No thermal treatment |
| treatment | | on a −20° C. shelf for 1 h | |
| Primary | The frozen solutions | After re-freezing the thermally - | The frozen solutions |
| drying | were primary dried on a | treated vials, primary drying was | were primary dried on a |
| | −10° C. shelf at a chamber | carried out for about 15 h at a | −15° C. shelf at a |
| | pressure of ~150 mTorr | shelf temperature of −15° C. and a | chamber pressure of |
| | for 14 h | chamber pressure of 150 mTorr. | about 150 mTorr for 16 h |

| | |
|---|---|
| Secondary | At the end of primary drying, the shelf was heated to +25° C. (about 30-40 min) and |
| drying | removal of sorbed water (secondary drying) was carried out for about 4 h |
| Stoppering | Vials were stoppered under negative pressure (about 150 mTorr). |

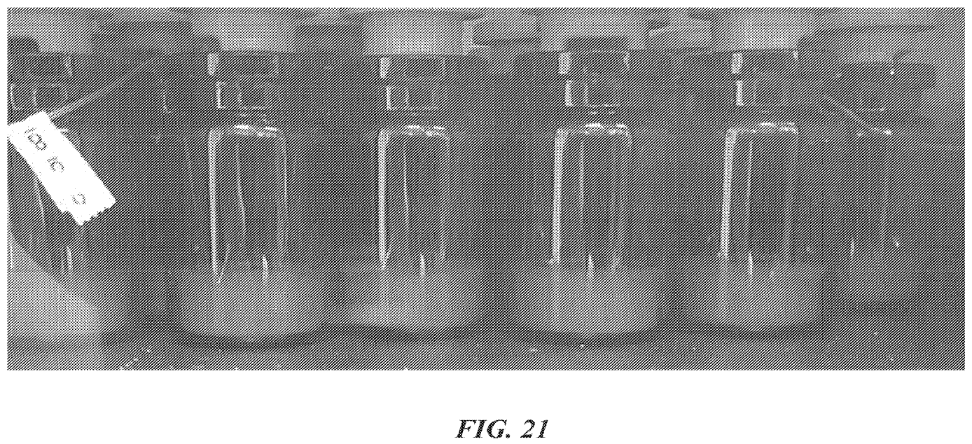

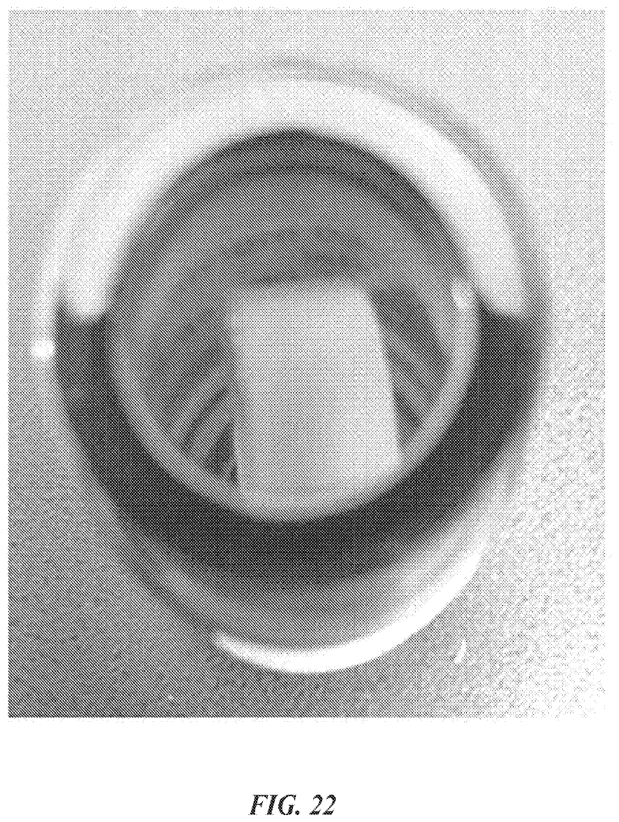

All of the Samples 10-1 through 10-3 resulted in elegant, lyophilized cakes with no evidence of collapse. FIGS. 21 and 22 are images of lyophilized products from ALM-488 sample formulations 10-1 and 10-2. FIG. 21 shows side view of lyophilized cakes, and FIG. 22 shows a top-down view of the dislodged lyophilized cake of ALM-488 samples formulation 10-2.

### C. Water Content of Lyophilized Samples

The lyophile water content of Samples 10-1 thru 10-3 was measured using a Mettler Toledo DL39 coulometric Karl Fischer titrator with a Mettler Toledo Stromboli automatic oven sample changer. The instrument was standardized with a Hydranal® Water standard (Fluka). Solid lyophile samples were weighed into sample vials and heated to 100° C. in the oven, and the water vaporized from the sample was transferred to the titrator cell by the purge gas. The measured water contents in the lyophile batches are tabulated in Table 15 below. Water contents were low and confirmed the suitability of the lyophilization process. The mannitol-containing lyophile (10-2) had residual water contents higher than the other two batches.

TABLE 15
Water Content in Lyophilized ALM-488 Formulation Samples

| | Lyophile water |
|---|---|
| Sample | content by KFT |
| 10-1 | 0.82, 0.75% w/w |
| (Arginine) | |
| 10-2 | 1.02, 1.11% w/w |
| (Arginine-Mannitol) | |
| 10-3 | 0.66, 0.64, 0.64, |
| (Arginine-Trehalose) | 0.75% w/w |

### D. Powder X-Ray Diffraction

X-ray diffraction patterns of the freeze-dried solid products of ALM-488 formulation samples 10-1, 10-2 and 10-3 were recorded on a wide-angle X-ray diffractometer (Bruker D8 Advance). The diffraction patterns were recorded over an angular range of 5 to 45° 20. The scans were recorded in the step scan mode with a step size of 0.005° 20 and a counting time of 0.25 sec/step.

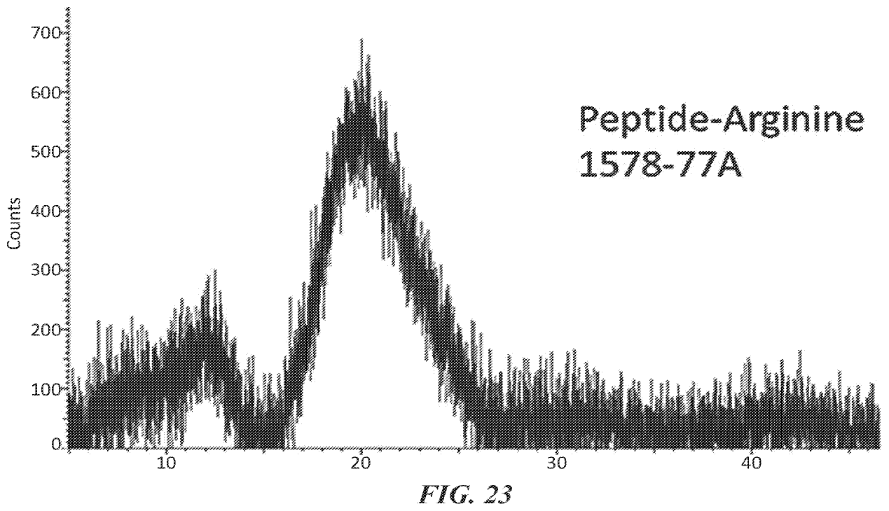

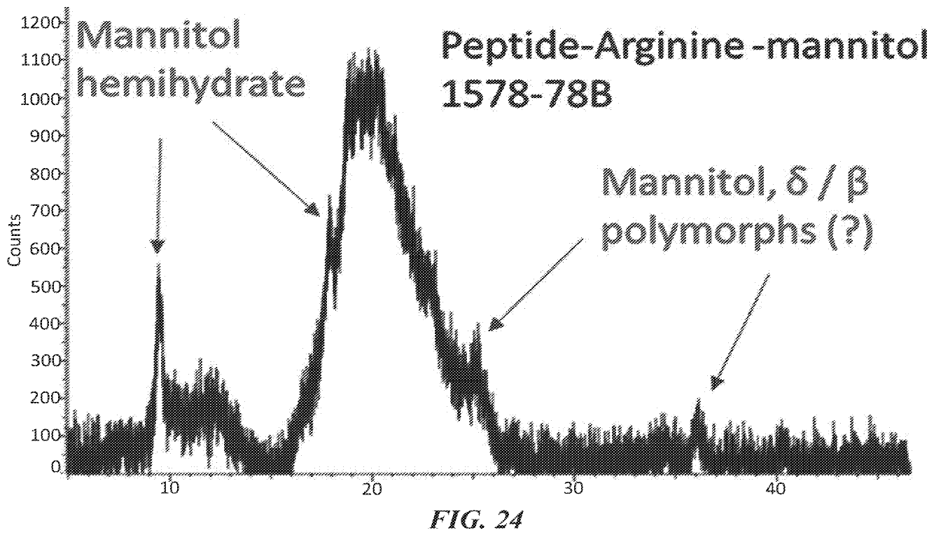

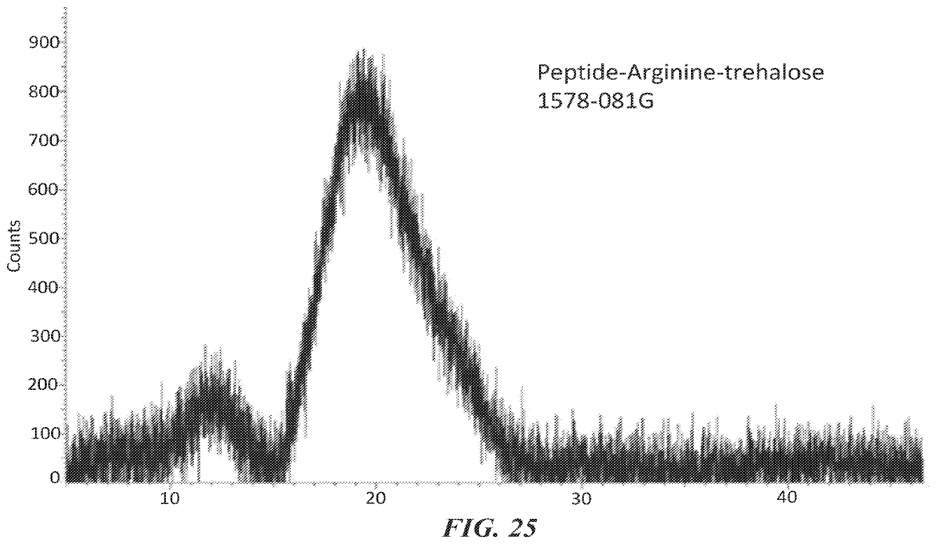

As shown in FIGS. 23 and 25, the PXRD patterns of the lyophiles of Samples 10-1 (arginine) and 10-3 (arginine-trehalose) exhibited an amorphous halo. By contrast, as shown in FIG. 24, the PXRD pattern of the lyophile of Sample 10-2 (arginine-mannitol) included characteristic peaks of crystalline mannitol. There was a clear evidence of mannitol hemihydrate along with peaks characteristic of mannitol δ- and β-anhydrate forms. The presence of mannitol hemihydrate may explain, in part, the higher residual water content in the lyophiles of Sample 10-2 in Table 15.

The PXRD pattern of Sample 10-2 in FIG. 24 suggests partial crystallization of mannitol during cooling and/or subsequent thermal treatment. However, the mannitol hemihydrate survives complete dehydration during secondary drying and is retained in the final lyophile. It has been reported that higher temperature drying steps are required to dehydrate mannitol hemihydrate formed during freeze-drying.

### E. Drug Product Assay

The content and purity of the ALM-488 peptide in the lyophilized product vials for Samples 10-1, 10-2 and 10-3 were evaluated using the HPLC method conditions of Table 3. To each vial of lyophilized product, 1 mL of purified water was added to achieve complete dissolution of the vial contents. The resulting solution was then transferred to a 200 mL volumetric flask. Each vial was rinsed well with water, and the rinses were transferred to the flask prior to volume make-up to 200 mL with water. The resulting solutions (~0.25 mg/mL of ALM488) were individually injected into the HPLC system and were analyzed as described above. The lyophilization process appeared to cause a small increase in the levels of some impurities. As shown in Table 16, impurities that were previously observed to form in solution (see RRT ~0.73, RRT ~1.16 and RRT ~1.20 in Tables 6 and 8-11) were also found in the lyophile products of Samples 10-1 thru 10-3.

TABLE 16
HPLC Data for Lyophile Samples 10-1 thru 10-3
versus HPLC Data for Starting ALM-488 peptide

| | Sample | | | |
|---|---|---|---|---|
| | | 10-1 | 10-2 | 10-3 |
| | Pre-Lyo. | (Arginine) | (Arginine-Mannitol) | (Arginine-Trehalose) |
| Component | Area % | | | |
| RRT~0.73 | — | 0.06 | 0.11 | — |
| | | 0.08 | 0..06 | |
| RRT~0.97 | 0.14 | 0.12 | 0.11 | 0.15 |
| | | 0.12 | 0.10 | 0.11 |
| ALM-488 | 99.69 | 99.32 | 99.25 | 99.43 |
| | | 99.29 | 99.20 | 99.53 |
| RRT~1.06 | 0.11 | 0.12 | 0.12 | 0.12 |
| | | 0.11 | 0.12 | 0.11 |
| RRT~1.16 | — | 0.13 | 0.13 | 0.10 |
| | | 0.12 | 0.12 | 0.07 |
| RRT~1.20 | — | 0.03 | 0.03 | — |
| | | 0.07 | 0.07 | |
| RRT~1.38 | 0.02 | 0.02 | 0.02 | 0.03 |
| | | 0.01 | 0.04 | 0.05 |
| Assay | NA | 49.4 mg/vial | 50.1 mg/vial | 51.3 mg/vial |
| ALM488 | | 49.7 mg/vial | 50.9 mg/vial | 52.4 mg/vial |
| mg/vial | | | | |

### F. Reconstituted Solution Compositions

Reconstituted solution compositions were then prepared using Samples 10-1, 10-2 and 10-3 in which the target reconstituted solution concentration is a 50 mg/mL solution obtained by reconstituting the contents of lyophilized samples to a final volume of 1 mL. The compositions of the reconstituted solutions are summarized in Table 17 below. Solutions of these compositions were prepared volumetrically, and their weights were used to estimate a density of the reconstituted solutions (listed in Table 17). This allowed for determination of the theoretical quantity of water to be added to each vial in order to obtain a reconstituted solution volume of 1 mL/vial and hence an ALM-488 concentration of 50 mg/mL.

TABLE 17
ALM488 reconstituted solution compositions.

| | 10-1 | 10-2 | 10-3 |
|---|---|---|---|
| Ingredient | (Arginine) | (Arginine-Mannitol) | (Arginine-Trehalose) |
| ALM488 (HCl) | 50 mg | 50 mg | 50 mg |
| L-Arginine USP | 18.8 mg | 18.8 mg | 18.8 mg |
| Mannitol USP | — | 27.2 mg | — |
| Trehalose (as dihydrate) | — | — | 56.4 mg¹ |
| Sterile Water for Injection | 960 mg | 930 mg | 930 mg |
| (qs to 1 mL/vial) ² | | | |
| Calculated density | 1.03 g/cc | 1.03 g/cc | 1.03 g/cc |

¹Dehydration and amorphization of trehalose dihydrate by freeze-drying would result in a theoretical quantity of 50.4 mg of anhydrous trehalose in the final freeze-dried vial. The original solution is made with 56.4 mg of trehalose dihydrate per vial.
² The quantity of water is based on the theoretical amount required to obtain a final reconstituted solution volume of 1 mL.

### G. Reconstitution of Lyophilized Samples

Lyophilized solids from Samples 10-1, 10-2 and 10-3 were reconstituted with fixed additions of reconstitution fluid (as summarized in Table 18). Lyophilized compositions for Samples 10-2 and 10-3 contained quantities of mannitol or trehalose calculated to yield osmolality values similar to human plasma upon reconstitution with water. The lyophilized composition for Sample 10-1 did not include a tonicity adjustment, and was reconstituted with normal saline (NS; 0.9% NaCl) or half-normal saline (HNS; 0.45% NaCl). Based on the osmolality of the pre-lyophilization compositions (see Table 7, Samples 8-1, 8-4 and 8-7), as expected, an acceptable osmolality was achieved when reconstituted with 0.45% NaCl.

TABLE 18
Reconstitution behavior of the lyophilized solids
and properties of the reconstituted solutions

| Reconstituted | 10-1 | 10-2 | 10-3 |
|---|---|---|---|
| Sample | (Arginine) | (Arginine-Mannitol) | (Arginine-Trehalose) |
| Reconstitution fluid | 0.9% NaCl (NS) | Sterile water for | Sterile water for |
| | 0.45% NaCl (HNS) | injection | injection |
| Fluid volume added | 0.96 mL | 0.93 mL | 0.93 mL |
| Recon. Solution | HNS - 276, 278 | 306, 306 | 310, 310 |
| osmolality | NS - 413, 420 | | |
| (mOsm/kg) | | | |
| Recon. Solution pH | HNS - 7.66 | 7.65 | 7.38 |
| | NS- 7.64 | | |

For all of Samples 10-1, 10-2 and 10-3, the lyophilized solid dissolved immediately yielding a clear solution in less than 1 minute after addition of the reconstitution fluid.

### H. HPLC Analsyis and Stability of Reconstituted Samples

The reconstituted solutions for Samples 10-1, 10-2 and 10-3, prepared as described above, were analyzed by HPLC using the method conditions of Table 3 to determine the ALM-488 content and to assess the level of impurities. Aliquots of these samples were also stored at 5° C. and 25° C., and where then analyzed after 24 h to confirm the stability of reconstituted solutions on short term storage. Tables 19A, 19B and 19C contain the HPLC data for the reconstituted samples obtained by reconstituting the lyophiles of Samples 10-1, 10-2 and 10-3 immediately on reconstitution and after storage. As shown in Tables 19A, 19B and 19C, there was no significant detectable growth of any impurities on short term storage of the reconstituted samples.

TABLE 19A
HPLC Analysis of Reconstituted Sample 10-1 Initially and After
Storage for 24 hours at 5° C. and 25° C.

| | Sample | | |
|---|---|---|---|
| | 10-1 | | |
| | (Arginine) | | |
| | (Reconstituted with 0.45% NaCl) | | |
| | Condition | | |
| | | 24 h , | 24 h, |
| | Initial | 5° C. | 25° C. |
| Component | AREA % | | |
| RRT~0.72 | 0.06 | 0.07 | 0.07 |
| | 0.04 | | |
| RRT ~0.93 | 0.01 | — | — |
| | 0.02 | | |
| RRT ~0.94 | 0.03 | — | 0.01 |
| | 0.03 | | |
| RRT ~0.97 | 0.11 | 0.12 | 0.10 |
| | 0.11 | | |
| ALM-488 | 99.37 | 99.34 | 99.42 |
| | 99.22 | | |
| RRT ~1.06 | 0.12 | 0.13 | 0.11 |
| | 0.16 | | |
| RRT ~1.11 | — | | |
| RRT ~1.13 | 0.08 | 0.06 | 0.07 |
| | 0.11 | | |
| RRT ~1.16 | 0.14 | 0.14 | 0.13 |
| | 0.15 | | |
| RRT ~1.18 | — | — | — |
| RRT ~1.38 | 0.02 | 0.03 | 0.05 |
| | 0.04 | | |
| ALM488 | 48.43 | 49.30 | 49.21 |
| (mg/mL) | 48.49 | | |

TABLE 19B
HPLC Analysis of Reconstituted Sample 10-2 Initially and After
Storage for 24 hours at 5° C. and 25° C.

Sample
10-2
(Arginine-Mannitol)
(Reconstituted with Sterile water for injection)
Condition

| | Initial | | |
|---|---|---|---|
| Component | AREA % | 24 h, 5° C. | 24 h 25° C. |
| RRT~0.72 | 0.05 | 0.07 | 0.06 |
| | 0.05 | | |
| RRT ~0.93 | 0.01 | 0.01 | — |
| | 0.02 | | |
| RRT ~0.94 | 0.01 | 0.03 | 0.01 |
| | 0.03 | | |
| RRT ~0.97 | 0.09 | 0.12 | 0.10 |
| | 0.10 | | |
| ALM-488 | 99.44 | 99.40 | 99.42 |
| | 99.39 | | |
| RRT ~1.06 | 0.12 | 0.12 | 0.12 |
| | 0.12 | | |
| RRT ~1.11 | — | — | |
| RRT ~1.13 | 0.05 | 0.06 | 0.06 |
| | 0.09 | | |
| RRT ~1.16 | 0.13 | 0.15 | 0.14 |
| | 0.15 | | |
| RRT ~1.18 | — | | |
| RRT ~1.38 | 0.03 | 0.04 | 0.03 |
| | 0.03 | | |
| ALM488 | 51.43 | 50.89 | 50.09 |
| (mg/mL) | 50.62 | | |

TABLE 19C
HPLC Analysis of Reconstituted Sample 10-3 Initially and After
Storage for 24 hours at 5° C. and 25° C.

Batch
10-3
(Arginine-Trehalose)
(Reconstituted with Sterile water for injection)
Condition

| | | |
|---|---|---|
| Initial | 24 h, 5° C. | 24 h, 25° C. |

| | |
|---|---|
| Component | AREA % |

| | | | |
|---|---|---|---|
| RRT~0.72 | — | — | — |
| RRT ~0.93 | 0.02 | 0.01 | 0.01 |
| | 0.01 | | |
| RRT ~0.94 | 0.04, 0.03 | 0.03 | |
| RRT ~0.97 | 0.11, 0.12 | 0.12 | 0.10 |
| ALM-488 | 99.53 | 99.50 | 99.40 |
| | 99.44 | | |
| RRT ~1.06 | 0.13 | 0.12 | 0.11 |
| | 0.12 | | |
| RRT ~1.11 | 0.04 | 0.04 | 0.04 |
| RRT ~1.13 | 0.05 | 0.08 | 0.09 |
| | 0.09 | | |
| RRT ~1.16 | 0.10 | 0.11 | 0.17 |
| | 0.10 | | |
| RRT ~1.18 | | | |
| RRT ~1.38 | 0.03 | 0.03 | 0.05 |
| | 0.05 | | |
| ALM488 | 51.4 | 52.1 | 52.6 |
| (mg/mL) | 52.2 | | |

### Example 11: Batch Formulations Containing ALM-488/Arginine-Trehalose

### A. Preparation of ALM-488/Arginine-Trehalose Lyophiles (Sample 11-1)

A series of batch formulations of ALM-488, HCl salt (prepared under the conditions of Examples 1 and 2) containing Arginine-Trehalose were prepared as summarized in Table 20. Quantities in Table 20 are expressed as amounts per viable for a 50 mg/vial ALM-488 product, and the ALM-488 concentration in the pre-lyophilization solutions is 25 mg/mL. The batch formulations are collectively referred to as Sample 11-1. The chloride content in the ALM-488, HCl salt was 3.67% (expressed as HCl). After correcting for water, this resulted in a peptide: HCl molar ratio of 1:2.5.

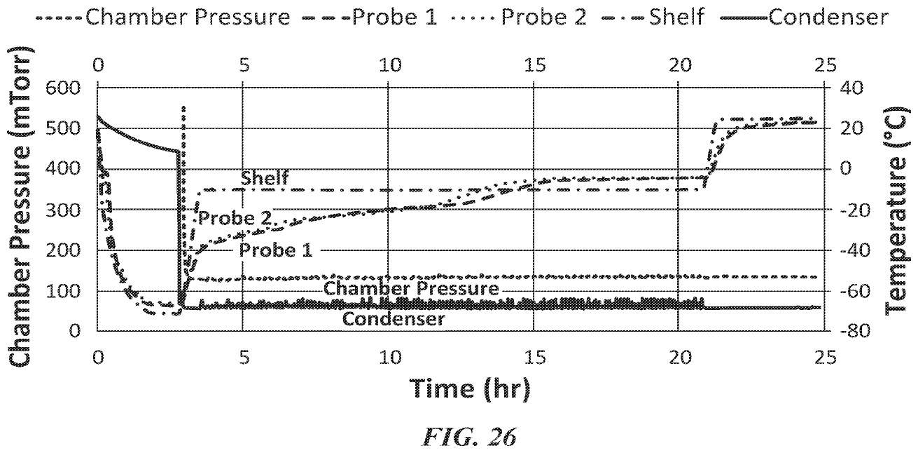

A batch solution was prepared as described above, filtered through a 0.22 micron PVDF filter, and then filled into a series of amber glass 5 mL vials (2 mL/vial) which were then freeze dried. The freeze-drying process used for the Sample 10-3 described above was used to prepare the lyophile samples 11-1, except that a primary drying shelf temperature of −10° C. was carried out for 17 hours, a 30 min ramp to a secondary drying temperature of 25° C. was carried out, and a 3.5 h secondary drying was carried out on a +25° C. shelf. The chamber pressure was maintained at ~150 m Torr during both the primary and secondary drying stages. A process graph for the freeze-drying process of Sample 11-1 is shown in FIG. 26.

TABLE 20
Lyophile of ALM-488 /Arginine-Trehalose Formulation

| Ingredient | Sample 11-1 | |
|---|---|---|
| ALM488 (HCl) ¹ | 50 | mg |
| L-Arginine USP | 18.8 | mg |
| Trehalose dihydrate USP | 48.6 | mg |
| Hydrochloric acid, 0.1N | qs to pH 7.5 ± 0.2 | |
| Sterile Water for Injection, | qs to 2.04 g | |
| (qs to 2 mL/vial) ² | | |
| 5 mL amber glass, 20 mm opening, | 1 each | |
| USP Type I tubing glass vials | | |
| 20 mm lyo stoppers | 1 each | |
| Flip-off aluminum crimp seal, 20 mm | 1 each | |
| Pre-lyophilization solution density | 1.02 | g/cc |
| Batch Size | 25 | vials |

| | |
|---|---|
| Solution pH | 7.63 |

¹ Chloride content (3.57% w/w; 3.67% w/w as HCl) and water content −4.08%, by KFT, peptide purity - 98.95% 50 mg of ALM488 = (50 × 100)/(0.9895) × (100 −4.08 −3.67) = 54.78 mg of drug substance.
² The density of the pre-lyophilization compositions was determined to be 1.02 g/cc. Using this density, the quantity of total solution fill per vial was calculated as listed in the table. Water is removed during processing.

### B. Reconstitution of ALM-488/Arginine-Trehalose Formulation (Sample 11-2)

A 1 mL volume per vial (50 mg/mL) of ALM-488/arginine-trehalose reconstituted formulation, designated as Sample 11-2, was prepared from Sample 11-1. Addition of 920 μL of sterile water to a vial of Sample 11-1 (50 mg/vial) resulted in a reconstituted formulation having the composition and properties in Table 21, where the concentration of the ALM-488 peptide is 50 mg/mL.

TABLE 21
Reconstituted ALM-488 /Arginine-Trehalose Formulation

| | Sample 11-2 | | | |
|---|---|---|---|---|
| Ingredient | Qty per 10 mL | | Equivalent qty per vial | |
| ALM488 (HCl)¹ | 500 | mg¹ | 50 | mg¹ |
| L-Arginine USP | 188 | mg | 18.8 | mg |
| Trehalose (as dihydrate)² | 486 | mg² | 48.6 | mg² |

| | | |
|---|---|---|
| Sterile Water for Injection | qs to 10 mL³ | qs to 1 mL³ |
| (qs to 1 mL/vial)³ | | |

| | |
|---|---|
| Measured density | 1.038 g/cc |

¹Chloride content (3.57% w/w; 3.67% w/w as HCl) and water content 4.08%, by KFT, peptide purity -98.95% 50 mg of ALM488 = (50 × 100)/(0.9895) × (100 −4.08 −3.67) = 54.78 mg of drug substance.
²Dehydration of trehalose dihydrate by freeze-drying would result in a theoretical quantity of 44 mg of anhydrous trehalose in the final freeze-dried vial.
³Based on the measured density, the amount of water in the 50 mg/mL solution is 920 mg (920 μL) per mL. This quantity of water added to a freeze-dried vial should theoretically yield a 1 mL solution and a 50 mg/mL ALM488 concentration.

### Example 12: Filtration Suitability Study Using ALM-488/Arginine-Trehalose

To evaluate whether filtration of a pre-lyophilized ALM-488/Arginine-Trehalose formulation is necessary or advantageous, both filtered and unfiltered ALM-488/Arginine-Trehalose sample pre-lyophilization samples were prepared in the same manner as described in Example 11. The unfiltered pre-lyophilization sample is designated as Sample 12-1, and the filtered pre-lyophilization samples is designated as Sample 12-2. The Sample 12-2 was filtered through a 0.22-micron Millipak Durapore® capsule filter unit, after the sample formulation was visually observed to be fully dissolved.

Samples 12-1 and 12-2 were analyzed by HPLC for drug content and purity using the HPLC method conditions of Table 3. Table 22 lists the levels (area %) of the main peak, key drug related impurities, as well as the calculated drug concentration in solution. As seen in the Table 22, there was no significant difference either in the levels of the impurities or in the dug content in solution (bottom row), between the unfiltered (12-1) and the filtered (12-2) solution samples. This data indicates that complete dissolution of the drug occurs during the formulation process, and that no obvious gross incompatibility exists with the filter material of the 0.22-micron Millipak Durapore® capsule filter unit.

TABLE 22
Chromatographic data for Samples 12-1
(unfiltered) and 12-2 (filtered)

Sample

| | |
|---|---|
| 12-1 | 12-2 |
| (unfiltered) | (filtered) |

| | |
|---|---|
| Component | AREA % |

| | | |
|---|---|---|
| RRT ~0.73 | 0.06 | 0.05 |
| RRT ~0.90 | 0.05 | 0.04 |
| RRT ~ 0.92 | 0.08 | 0.08 |
| RRT ~0.94 | 0.13 | 0.12 |
| ALM-488 | 99.32 | 99.28 |
| RRT~ 1.06 | 0.37 | 0.39 |
| ALM-488 Assay (mg/mL) | 25.9 | 26.1 |

### Example 13: Long-Term Stability of ALM-488 Lyophiles

Lyophilized ALM-488 samples 13-1 (arginine), 13-2 (arginine-mannitol) and 13-3 (arginine-trehalose), which were prepared in a manner identical to the corresponding samples 10-1 (arginine), 10-2 (arginine-mannitol) and 10-3 (arginine-trehalose) of Example 10, were stored at various temperatures and various relative humidities for periods of up to three months. Then the resulting lyophile Samples 13-1, 13-2 and 13-3 were reconstituted (hydrated) and tested for purity using the HPLC method conditions of Table 3. The lyophilized ALM-488/Arginine-Trehalose Sample 13-3 was also tested for water content using Karl Fischer titration and was subjected to PXRD analysis.

### A. Effect of Long-Term Storage on HPLC Purity of Lyophilized ALM-488/Arginine (Sample 13-1)

The lyophilized ALM-488/Arginine Sample 13-1 (corresponding to Sample 10-1 of Example 10) was divided into three HPLC vials which were stored (a) at 40° C. and 75% relative humidity for three months (Sample 13-1-a), (b) at 25° C. and 60% relative humidity for three months (Sample 13-1-b), and (c) at 5° C. for three months (Sample 13-1-c). Then each sample was tested for purity using the HPLC method conditions of Table 3. Table 23 below summarizes the HPLC purity data for the Samples 13-1-a, 13-1-b and 13-1-c compared to the initial (pre-storage) Sample 13-1. As shown in Table 23, the overall extent of degradation in all the stored samples was low (<0.6% loss of intact drug after 3 months at 40° C./75% RH).

TABLE 23
Chromatographic data for ALM-488/Arginine samples
stored at different temperatures and relative humidities
for 3 Months
HPLC Analyses of Lyophilized ALM-488/Arginine Samples

| Sample | 13-1 | 13-1-a | 13-1-b | 13-1-c |
|---|---|---|---|---|
| Storage | Initial | 40° C./7 | 25° C./ | 5º C. |
| Condition | | 5% RH | 60% RH | 3 months |
| | | 3 months | 3 months | |
| Component | Area % | | | |
| RRT~0.72 | 0.06 | 0.06 | 0.06 | 0.04 |
| | 0.08 | 0.08 | 0.07 | 0.05 |
| RRT~0.94 | — | — | — | 0.02 |
| | | | | 0.02 |
| RRT~0.97 | 0.12 | 0.17 | 0.15 | 0.09 |
| | 0.12 | 0.15 | 0.12 | 0.09 |
| ALM-488 | 99.32 | 98.98 | 99.52 | 99.50 |
| | 99.29 | 98.89 | 99.47 | 99.41 |
| RRT~1.06 | 0.12 | 0.19 | 0.14 | 0.13 |
| | 0.11 | 0.16 | 0.14 | 0.15 |
| RRT~1.11 | — | 0.10 | 0.05 | 0.07 |
| | | 0.12 | 0.07 | 0.12 |
| RRT~1.13 | — | 0.10 | 0.03 | 0.04 |
| | | | 0.06 | 0.06 |
| RRT~1.16 | 0.13 | 0.14 | 0.08 | 0.13 |
| | 0.12 | 0.20 | 0.06 | 0.14 |
| RRT~1.30 | — | 0.16 | — | — |
| | | 0.12 | | |
| RRT~1.38 | 0.02 | 0.07 | — | — |
| | 0.01 | — | | |
| Total | 0.68 | 1.02 | 0.48 | 0.50 |
| Impurities | 0.71 | 1.11 | 0.53 | 0.59 |

### B. Effect of Long-Term Storage on HPLC Purity of Lyophilized ALM-488/Arginine-Mannitol (Sample 13-2)

The lyophilized ALM-488/Arginine-Mannitol Sample 13-2 (corresponding to Sample 10-2 of Example 10) was divided into three HPLC vials which were stored (a) at 40° C. and 75% relative humidity for three months (Sample 13-2-a), (b) at 25° C. and 60% relative humidity for three months (Sample 13-2-b), and (c) at 5° C. for three months (Sample 13-2-c). Then, each sample was tested for purity using the HPLC method conditions of Table 3. Table 24 below summarizes the HPLC purity data for the Samples 13-2-a, 13-2-b and 13-2-c compared to the initial (pre-storage) Sample 13-2. As shown in Table 24, in all samples the proportion of measured ALM-488 appeared to increase following storage. However, the presence of mannitol hemihydrate in the Sample 10-2 is believed to pose a potential risk for dehydration during extended storage and consequent stability implications.

TABLE 24
Chromatographic data for ALM-488 / Arginine-Mannitol samples
stored at different temperatures and relative humidities for 3 Months
HPLC Analyses of Lyophilized ALM-488 / Arginine-Mannitol Samples

| | Sample | | | |
|---|---|---|---|---|
| | 13-2 | 13-2-a | 13-2-b | 13-2-c |
| | Storage Condition | | | |
| | | 40° C. / | 25° C. / | |
| | | 75% RH | 60% RH | 5° C. |
| | Initial | 3 months | 3 months | 3 months |
| Component | Area % | | | |
| RRT ~0.72 | 0.11 | 0.06 | 0.04 | 0.05 |
| | 0.06 | 0.04 | 0.05 | 0.04 |
| RRT ~0.94 | — | 0.03 | 0.04 | 0.04 |
| | | 0.02 | 0.01 | 0.02 |
| RRT ~0.97 | 0.11 | 0.09 | 0.11 | 0.12 |
| | 0.10 | 0.08 | 0.07 | 0.08 |
| ALM-488 | 99.25 | 99.55 | 99.48 | 99.40 |
| | 99.20 | 99.60 | 99.66 | 99.60 |
| RRT ~1.06 | 0.12 | 0.13 | 0.14 | 0.16 |
| | 0.12 | 0.12 | 0.12 | 0.12 |
| RRT ~1.11 | — | 0.04 | 0.04 | 0.07 |
| | | 0.05 | 0.04 | 0.12 |
| RRT ~1.13 | — | 0.04 | 0.05 | 0.04 |
| | | 0.03 | 0.01 | 0.03 |
| RRT ~1.16 | 0.13 | 0.09 | 0.07 | 0.10 |
| | 0.12 | 0.07 | 0.05 | 0.08 |
| RRT ~1.38 | 0.02 | — | — | — |
| | 0.04 | | | |
| Total Impurities | 0.75 | 0.45 | 0.52 | 0.60 |
| | 0.80 | 0.40 | 0.34 | 0.40 |

### C. Effect of Long-Term Storage on HPLC Purity of Lyophilized ALM-488/Arginine-Trehalose (Sample 13-3)

The lyophilized ALM-488/Arginine-Trehalose Sample 13-3 (corresponding to Sample 10-3 of Example 10) was divided into three HPLC vials which were stored (a) at 25° C. and 60% relative humidity for one month (Sample 13-3-a), (b) at 25° C. and 60% relative humidity for two months (Sample 13-3-b), (c) at 25° C. and 60% relative humidity for three months (Sample 13-3-c), (d) at 5° C. for two months (Sample 13-2-d), (e) at 5° C. for three months (Sample 13-2-e), (f) at 40° C. and 75% relative humidity for one month (Sample 13-3-f), (g) at 40° C. and 75% relative humidity for two months (Sample 13-3-g), and (h) at 40° C. and 75% relative humidity for three months (Sample 13-3-h). Then each sample was tested for purity using the HPLC method conditions of Table 3. Table 25 below summarizes the HPLC purity data for the Samples 13-3-a, 13-3-b, 13-3-c, 13-3-d and 13-3-3e compared to the initial (pre-storage) Sample 13-3.

TABLE 25
Chromatographic data for ALM-488/Arginine-Trehalose samples
stored at different temperatures and relative humidities for 1-3 Months
HPLC Analyses of Lyophilized ALM-488/Arginine-Trehalose

| | Sample | | | | | |
|---|---|---|---|---|---|---|
| | | 13-3-a | 13-3-b | 13-3-c | 13-3-d | 13-3-e |
| | 13-3 | 25° C./ 60% RH | 25° C./ 60% RH | 25° C./ 60% RH | 5° C. | 5° C. |
| | Initial | 1 month | 2 months | 3 months | 2 months | 3 months |
| Component | Area % | | | | | |
| RRT~0.72 | — | 0.06 | 0.10 | 0.04 | 0.08 | 0.06 |
| | | 0.06 | 0.12 | 0.04 | 0.11 | 0.06 |
| RRT ~0.94 | 0.03 | 0.06 | 0.07 | 0.06 | 0.04 | 0.09 |
| | 0.05 | 0.06 | 0.08 | 0.03 | 0.07 | 0.08 |
| RRT~0.97 | 0.15 | 0.25 | 0.34 | 0.33 | 0.22 | 0.17 |
| | 0.11 | 0.23 | 0.32 | 0.41 | 0.23 | 0.16 |
| ALM-488 | 99.43 | 99.40 | 99.10 | 99.22 | 99.32 | 99.29 |
| | 99.53 | 99.44 | 99.15 | 99.16 | 99.19 | 99.27 |
| RRT~1.06 | 0.12 | 0.11 | 0.17 | 0.16 | 0.14 | 0.17 |
| | 0.11 | 0.10 | 0.14 | 0.14 | 0.15 | 0.17 |
| RRT~1.11 | 0.05 | 0.04 | 0.13 | 0.10 | 0.08 | 0.10 |
| | 0.04 | 0.04 | 0.07 | 0.10 | 0.07 | 0.10 |
| RRT~1.13 | 0.07 | — | — | 0.05 | 0.06 | 0.07 |
| | 0.08 | 0.03 | 0.06 | 0.05 | 0.09 | 0.07 |
| RRT~1.16 | 0.10 | 0.06 | 0.12 | 0.06 | 0.08 | 0.10 |
| | 0.07 | 0.05 | 0.08 | 0.08 | 0.11 | 0.11 |
| RRT~1.38 | 0.03 | 0.01 | — | — | — | — |
| | 0.05 | 0.01 | | | | |
| Total | 0.55 | 0.59 | 0.93 | 0.80 | 0.70 | 0.76 |
| Impurities | 0.51 | 0.58 | 0.87 | 0.85 | 0.83 | 0.75 |

Table 26 below summarizes the HPLC purity data for the Samples 13-3-f, 13-3-g and 13-3-h compared to the initial (pre-storage) Sample 13-3.

TABLE 26
Chromatographic data for ALM-488 / Arginine-Trehalose samples stored
at 40° C. / 75% RH for 3 months
HPLC Analyses of Lyophilized ALM-488 / Arginine-Trehalose

| | Sample | | | |
|---|---|---|---|---|
| | 13-3 | 13-3-f | 13-3-g | 13-3-h |
| | Storage Condition | | | |
| | | 40° C./ | 40° C./ | 40° C./ |
| | | 75% RH | 75% RH | 75% RH |
| | Initial | 1 month | 2 months | 3 months |
| Component | Area % | | | |
| RRT ~0.72 | — | 0.04 | 0.08 | 0.05 |
| | | 0.05 | 0.06 | 0.05 |
| RRT ~0.94 | 0.03 | 0.10 | 0.07 | 0.12 |
| | 0.05 | 0.12 | 0.10 | 0.13 |
| RRT ~0.97 | 0.15 | 0.37 | 0.65 | 0.54 |
| | 0.11 | 0.37 | 0.53 | 0.48 |
| ALM-488 | 99.43 | 99.26 | 98.83 | 98.83 |
| | 99.53 | 99.12 | 99.03 | 98.93 |
| RRT ~1.06 | 0.12 | 0.11 | 0.15 | 0.19 |
| | 0.11 | 0.15 | 0.15 | 0.17 |
| RRT ~1.11 | 0.05 | 0.04 | 0.08 | 0.10 |
| | 0.04 | 0.04 | 0.05 | 0.09 |
| RRT ~1.13 | 0.07 | 0.04 | 0.02 | 0.07 |
| | 0.08 | 0.04 | 0.03 | 0.06 |
| RRT ~1.16 | 0.10 | 0.07 | 0.14 | 0.12 |
| | 0.07 | 0.09 | 0.08 | 0.10 |
| RRT ~1.38 | 0.03 | — | — | — |
| | 0.05 | | | |
| Total Impurities | 0.55 | 0.77 | 1.19 | 1.19 |
| | 0.51 | 0.86 | 1.00 | 1.08 |

As shown in Tables 25 and 26, the overall extent of degradation in all the ALM-488/Arginine-Trehalose lyophiles was low (<0.6% loss of intact drug after 3 months at 40° C./75% RH). The main degradation product appeared as a shoulder on the main peak (RRT ~0.97) and grew in the samples especially at 40° C./75% RH and 25° C./60% RH conditions.

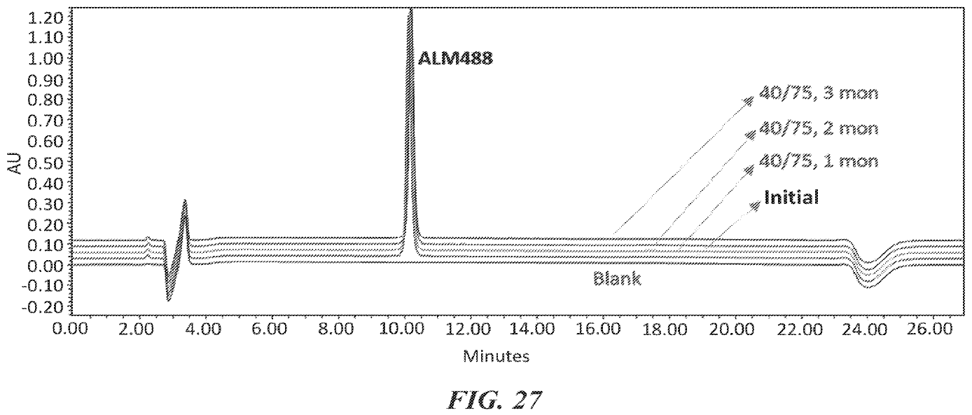

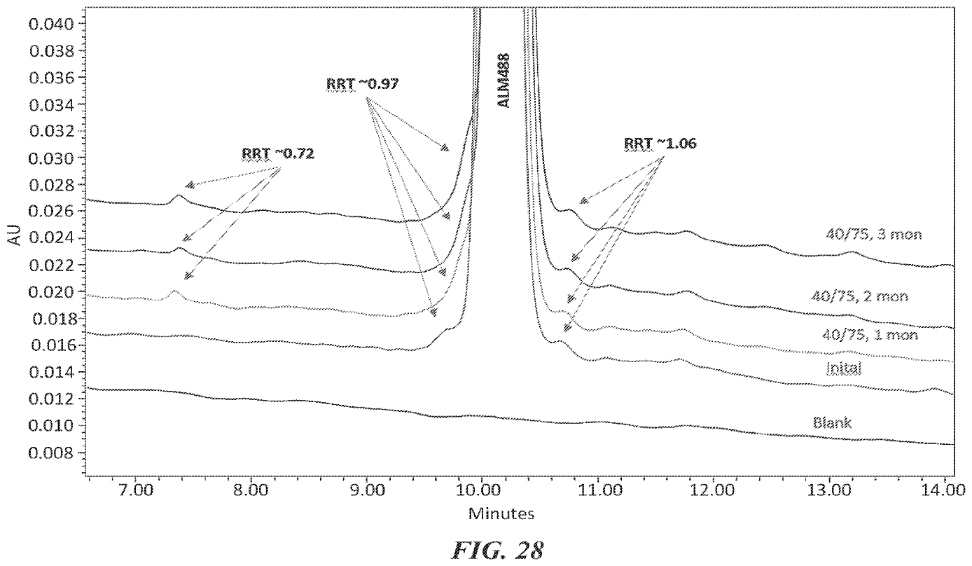

FIGS. 27 and 28 are overlays of the HPLC chromatograms for the Sample 13-3 (ALM-488/arginine-trehalose) lyophiles initially and after 1, 2 and 3 months of storage at 40° C./75% RH. The chromatograms show the growth of the primary impurity (RRT ~0.97) as a shoulder on the main peak during storage.

Comparing the long-term storage results for Samples 13-1 (arginine), 13-2 (arginine-mannitol) and 13-3 (arginine-trehalose), it is concluded that the arginine-trehalose sample 13-3 is the best candidate for further development. Sample 13-3 is advantageous over Sample 13-1 because use of the tonicity adjusting agent (trehalose) allows for fine tuning of the pH and solubility characteristics of the pre-lyophilized and reconstituted formulations. Despite Sample 13-2 exhibiting better long-term stability over a three-month period, Sample 13-3 is advantageous over Sample 13-2 because the presence of mannitol hemihydrate in the Sample 13-2 is believed to pose a potential risk for dehydration during extended storage and consequent stability implications.

### D. Effect of Long-Term Storage on Water Content of Sample 13-3

The ALM-488/Arginine-Trehalose Lyophile of Sample 13-3 was stored at 40° C. and 75% relative humidity for a period of three months, prior to being tested using the Karl Fischer titration methodology described above in Example 10. After this long-term storage, the water content of Sample 10-3 had increased from an initial value of ~0.7% w/w to ~1.8% w/w. This increase could be due to a combination of water transfer from the rubber stopper to the amorphous dry drug product, as well as water vapor transmission through the stopper from the humid external environment.

### E. Effect of Long-Term Storage on Powder X-Ray Diffraction of Sample 10-3

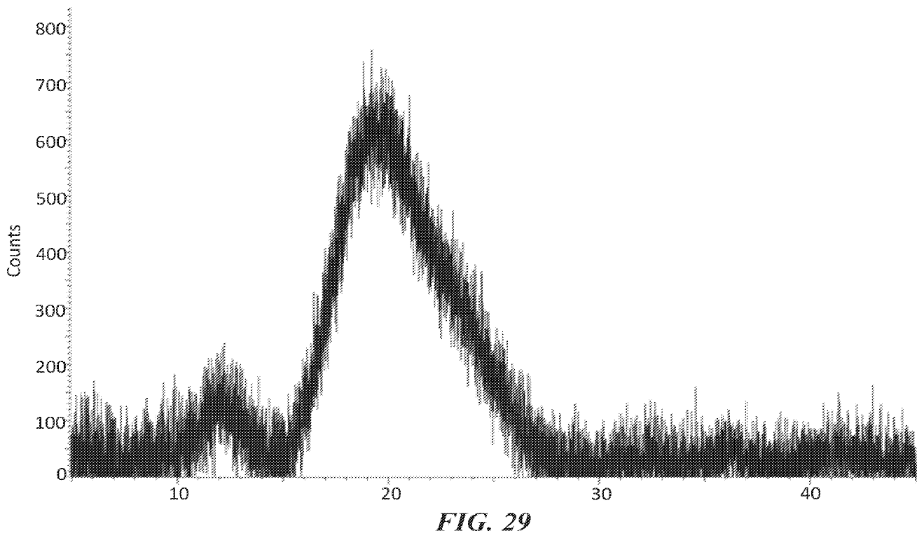

The ALM-488/Arginine-Trehalose Lyophile of Sample 13-3 was stored at 40° C. and 75% relative humidity for a period of three months, prior to being tested using the PXRD methodology described above in Example 10. As shown in FIG. 29, the lyophile of Sample 10-3 retained its amorphous character after being subjected to the storage at elevated temperature and humidity. Further, the resulting lyophile sample exhibited rapid dissolution into a clear solution within 1 min after addition of water to the sample vial.

These and other changes can be made to the embodiments in light of the above-detailed description. In general, in the following claims, the terms used should not be construed to limit the claims to the specific embodiments disclosed in the specification and the claims but should be construed to include all possible embodiments along with the full scope of equivalents to which such claims are entitled. Accordingly, the claims are not limited by the disclosure. The present application claims priority to U.S. Provisional Application No. 63/501,593 filed on May 11, 2023 in the United States Patent Office, the entire contents and disclosure of which are incorporated herein by reference.

What is claimed is:

1. An aqueous pharmaceutical composition, comprising:
(a) a fluorescein-labeled nerve targeting peptide of formula (I): 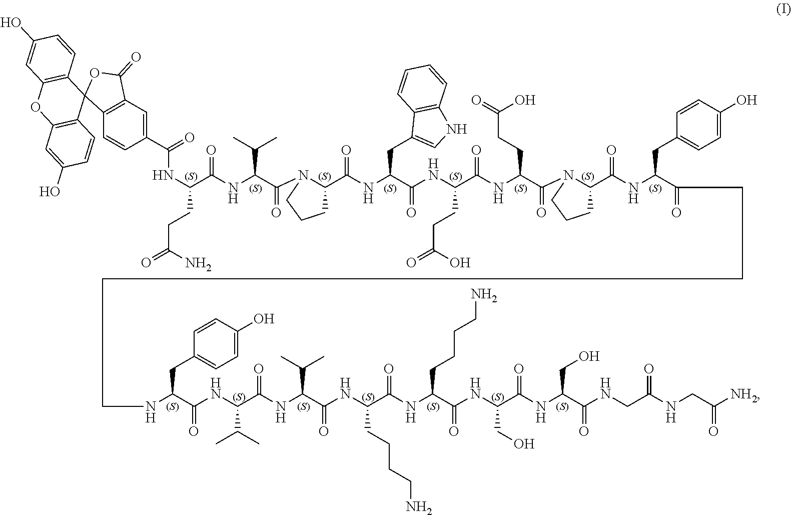
or pharmaceutically acceptable salt, solvate, hydrate, isomer, tautomer, racemate, or isotope thereof;
(b) an additive comprising a pH-adjusting agent, a buffering agent, a bulking agent, a tonicity-adjusting agent, a preservative, or any combination thereof; and
(c) an aqueous vehicle,
wherein:
a pH of the composition ranges from about 7 to about 9; and
a concentration of the peptide in the aqueous composition ranges from about 25 mg/mL to about 200 mg/mL, based on a total volume of the aqueous composition.

2. The aqueous composition according to claim 1, comprising a pharmaceutically-acceptable salt of the peptide.

3. The aqueous composition according to claim 1 or 2, comprising an HCl salt of the peptide.

4. The aqueous composition according to claim 3, wherein a chloride content of the HCl salt ranges from about 3.0% (w/w) to about 4.0% (w/w), based on a total mass of the peptide in the aqueous composition.

5. The aqueous composition according to any one of claims 2-4, wherein a molar ratio of the peptide to the pharmaceutically-acceptable salt ranges from about 1:1 to about 1:3.

6. The aqueous composition according to any one of claims 1-5, wherein the additive comprises a pH-adjusting agent, a bulking agent, a tonicity-adjusting agent, or any combination thereof.

7. The aqueous composition according to any one of claims 1-6, wherein the additive comprises alanine, arginine, a cellulose, cellobiose, a cyclodextrin, dextrose, erythritol, fructose, galactose, glucose, glycerin, glycine, histidine, inositol, lactose, maltose, mannitol, mannose, meglumine, raffinose, ribose, sorbitol, sucrose, trehalose, tromethamine, xylitol, xylose, or any combination thereof.

8. The aqueous composition according to any one of claims 1-6, wherein the additive comprises arginine, glycine, mannitol, meglumine, trehalose, tromethamine, or any combination thereof.

9. The aqueous composition according to any one of claims 1-8, wherein the additive comprises:
a first additive comprising arginine, meglumine, glycine, or any combination thereof; and
optionally a second additive comprising mannitol, trehalose, or both.

10. The aqueous composition according to claim 9, wherein a mass ratio of the first additive to the second additive ranges from about 1:1 to about 1:5.

11. The aqueous composition according to any one of claims 1-9, wherein the additive comprises arginine, arginine-mannitol, arginine-trehalose, arginine-meglumine, meglumine, meglumine-glycine, or meglumine-mannitol.

12. The aqueous composition according to any one of claims 1-11, wherein the additive comprises arginine-trehalose

13. The aqueous composition according to claim 12, wherein that a mass ratio of arginine to trehalose ranges from about 1:2 to about 1:3.

14. The aqueous composition according to any one of claims 1-13, wherein a mass ratio of the peptide to the additive ranges from about 1.0 to about 1.5.

15. The aqueous composition according to any one of claims 1-14, wherein the aqueous vehicle comprises a sterile water, a saline solution, an aqueous buffer, or any combination thereof.

16. The aqueous composition according to any one of claims 1-15, wherein the aqueous vehicle comprises a sterile water, a normal saline, a half-normal saline, or any combination thereof.

17. The aqueous composition according to any one of claims 1-16, wherein the pH of the aqueous composition ranges from about 7.2 to about 7.8.

18. The aqueous composition according to any one of claims 1-17, wherein the concentration of the peptide in the aqueous composition ranges from about 40 mg/mL to about 75 mg/mL.

19. The aqueous composition according to any one of claims 1-18, wherein a density of the aqueous composition ranges from about 0.85 g/mL to about 1.15 g/mL.

20. The aqueous composition according to any one of claims 1-19, wherein an osmolality of the aqueous composition ranges from about 200 mOsm/kg to about 350 mOsm/kg.

21. The aqueous composition according to any one of claims 1-20, wherein a volume of the aqueous composition ranges from about 1.8 mL to about 2.2 mL.

22. The aqueous composition according to any one of claims 1-21, wherein the aqueous composition is filtered.

23. The aqueous composition according to any one of claims 1-21, wherein the aqueous composition is non-filtered.

24. The aqueous composition according to any one of claims 1-23, which comprises at least 98.5 mass %, as measured relative to a total mass of the aqueous composition, after storage at 40° C. for a period of 14 days.

25. A lyophilized pharmaceutical composition formed by lyophilizing the aqueous composition according to any one of claims 1-24.

26. A lyophilized pharmaceutical composition, comprising:
(a) a fluorescein-labeled nerve targeting peptide of formula (I): 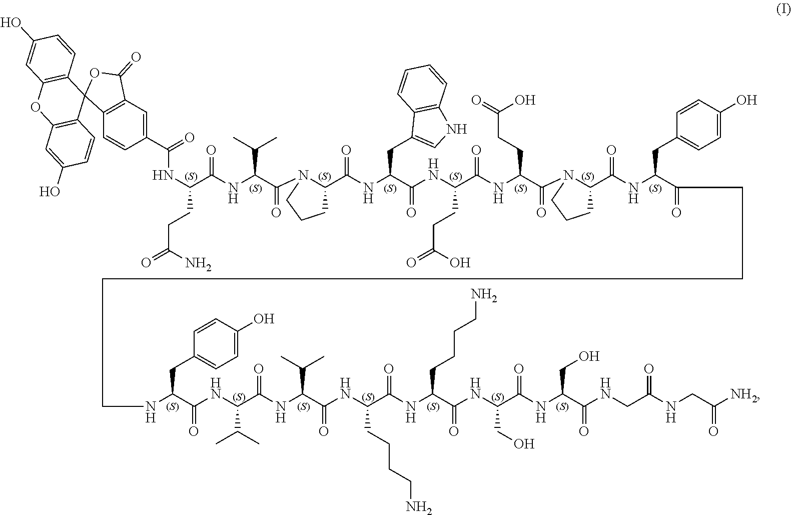
or pharmaceutically acceptable salt, solvate, hydrate, isomer, tautomer, racemate, or isotope thereof; and
(b) an additive comprising a pH-adjusting agent, a buffering agent, a bulking agent, a tonicity-adjusting agent, a preservative, or any combination thereof,
wherein a water content of the lyophilized composition ranges from about 0.2% (w/w) to about 2.0% (w/w), relative to a total mass of the lyophilized composition.

27. The lyophilized composition according to claim 26, wherein the lyophilized composition forms a homogeneous aqueous composition having a peptide concentration of 200 mg/mL or less when combined with an aqueous vehicle at ambient temperature.

28. The lyophilized composition according to claim 26 or 27, wherein the lyophilized composition is in the form of an elegant lyophilized cake.

29. The lyophilized composition according to any one of claims 26-28, wherein the lyophilized composition comprises at least 98.5 mass % of the peptide after storage of the lyophilized composition at 40° C. and 75% relative humidity for a period of 3 months.

30. The lyophilized composition according to any one of claims 26-29, comprising a pharmaceutically-acceptable salt of the peptide.

31. The lyophilized composition according to any one of claims 26-30, comprising an HCl salt of the peptide.

32. The lyophilized composition according to claim 31, wherein a chloride content of the HCl salt ranges from about 3.0% (w/w) to about 4.0% (w/w), based on a total mass of the HCl salt of the peptide.

33. The lyophilized composition according to claim 31 or 32, wherein a molar ratio of the peptide to the pharmaceutically-acceptable salt ranges from about 1:1 to about 1:3.

34. The lyophilized composition according to any one of claims 26-33, wherein the additive comprises a pH-adjusting agent, a bulking agent, a tonicity-adjusting agent, or any combination thereof.

35. The lyophilized composition according to any one of claims 26-34, wherein the additive comprises alanine, arginine, a cellulose, cellobiose, a cyclodextrin, dextrose, erythritol, fructose, galactose, glucose, glycerin, glycine, histidine, inositol, lactose, maltose, mannitol, mannose, meglumine, raffinose, ribose, sorbitol, sucrose, trehalose, tromethamine, xylitol, xylose, or any combination thereof.

36. The lyophilized composition according to any one of claims 26-35, wherein the additive comprises arginine, glycine, mannitol, meglumine, trehalose, tromethamine, or any combination thereof.

37. The lyophilized composition according to any one of claims 26-36, wherein the additive comprises a first additive comprising arginine, meglumine or glycine, and optionally a second additive comprising mannitol or trehalose.

38. The lyophilized composition according to claim 37, wherein a mass ratio of the first additive to the second additive ranges from about 1:1 to about 1:5.

39. The lyophilized composition according to any one of claims 26-38, wherein the additive comprises arginine, arginine-mannitol, arginine-trehalose, arginine-meglumine, meglumine, meglumine-glycine, or meglumine-mannitol.

40. The lyophilized composition according to any one of claims 26-39, wherein the additive comprises arginine-trehalose.

41. The lyophilized composition according to claim 40, wherein a mass ratio of arginine to trehalose ranges from about 1:2 to about 1:3.

42. The lyophilized composition according to any one of claims 26-41, wherein a mass ratio of the peptide to the additive ranges from about 1.0 to about 1.5.

43. A reconstituted aqueous pharmaceutical composition formed by combining the lyophilized composition according to any one of claims 26-42 with an aqueous vehicle.

44. The reconstituted aqueous composition according to claim 43, wherein the aqueous vehicle comprises a sterile water, a saline solution, an aqueous buffer, or any combination thereof.

45. The reconstituted aqueous composition according to claim 43, wherein the aqueous vehicle comprises a sterile water, a normal saline, a half-normal saline, or any combination thereof.

46. The reconstituted aqueous composition according to any one of claims 43-45, wherein the reconstituted composition is homogeneous.

47. The reconstituted aqueous composition according to any one of claims 43-46, wherein a pH of the reconstituted composition ranges from about 7 to about 9.

48. The reconstituted aqueous composition according to any one of claims 43-47, wherein a concentration of the peptide in the reconstituted composition ranges from about 25 mg/mL to about 200 mg/mL, based on a total volume of the reconstituted composition.

49. The reconstituted aqueous composition according to any one of claims 43-48, wherein a density of the reconstituted composition ranges from about 0.85 g/mL to about 1.15 g/mL.

50. The reconstituted aqueous composition according to any one of claims 43-49, wherein an osmolality of the reconstituted composition ranges from about 200 mOsm/kg to about 350 mOsm/kg.

51. The reconstituted aqueous composition according to any one of claims 43-50, wherein a volume of the reconstituted composition ranges from about 1.8 mL to about 2.2 mL.

52. The reconstituted aqueous composition according to any one of claims 43-51, wherein the reconstituted composition is filtered.

53. The reconstituted aqueous composition according to any one of claims 43-52, wherein the reconstituted composition is a non-filtered.

54. The reconstituted aqueous composition according to any one of claims 43-53, which comprises at least 98.5 mass % of the peptide, as measured relative to a total mass of the reconstituted composition, after storage at 40° C. for a period of 14 days.

55. A method for forming a lyophilized pharmaceutical composition, the method comprising lyophilizing an aqueous composition according to at least one of claims 1-24 to obtain a lyophile.

56. The method according to claim 55, wherein the lyophilizing comprises freezing the aqueous composition at a temperature ranging from about −80° C. to about −50° C. to obtain a frozen solid composition.

57. The method according to claim 56, wherein the lyophilizing further comprises annealing the frozen solid composition at temperature ranging from about −40° C. to about −20° C.

58. The method according to claim 56 or 57, wherein the lyophilizing further comprises drying the frozen solid composition at a temperature ranging from about −20° C. to room temperature, at a pressure ranging from about 100 mTorr to about 300 mTorr, to obtain the lyophilized composition.

59. The method according to according to claim 58, wherein the drying comprises:
a primary drying of the frozen solid composition at a temperature ranging from about −20° C. to about 0° C. at a primary pressure ranging from about 100 mTorr to about 300 mTorr; and
a secondary drying of the frozen solid composition at a temperature ranging from about 0° C. to room temperature at a secondary pressure ranging from about 100 mTorr to about 300 mTorr.

60. A lyophilized pharmaceutical composition formed by the method according to any one of claims 55-59.

61. A method for forming a reconstituted aqueous pharmaceutical composition, the method comprising combining a lyophilized composition according to any one of claims 26-42 with an aqueous vehicle.

62. The method according to claim 61, further comprising filtering the reconstituted aqueous composition.

63. The method according to claim 61, wherein the reconstituted aqueous composition is non-filtered.

64. The method according to any one of claims 61-63, wherein the reconstituted aqueous composition is homogeneous.

65. The method according to any one of claims 61-64, wherein the aqueous vehicle comprises a sterile water, a saline solution, an aqueous buffer, or any combination thereof.

66. The method according to any one of claims 61-64, wherein the aqueous vehicle comprises a sterile water, a normal saline, a half-normal saline, or any combination thereof.

67. The method according to any one of claims 61-66, wherein a pH of the reconstituted aqueous composition ranges from about 7 to about 9.

68. The method according to any one of claims 61-67, wherein a concentration of the peptide in the reconstituted aqueous composition ranges from about 25 mg/mL to about 200 mg/mL, based on a total volume of the reconstituted composition.

69. The method according to any one of claims 61-68, wherein a density of the reconstituted aqueous composition ranges from about 0.85 g/mL to about 1.15 g/mL.

70. The method composition according to any one of claims 61-69, wherein an osmolality of the reconstituted aqueous composition ranges from about 200 mOsm/kg to about 350 mOsm/kg.

71. The method according to any one of claims 61-70, wherein a volume of the reconstituted aqueous composition ranges from about 1.8 mL to about 2.2 mL.

72. The method according to any one of claims 61-71, wherein the reconstituted aqueous composition comprises at least 98.5 mass % of the peptide, as measured relative to a total mass of the reconstituted aqueous composition, after storage at 40° C. for a period of 14 days.

73. A method for identifying a human neuron or nerve, the method comprising contacting the human neuron or nerve with a composition according to any one of claims 1-54.

74. The method of claim 73, wherein the human neuron or nerve is contacted with a free fluorescent moiety in combination with the composition.

75. A method of delivering a drug to a human neuron or nerve, the method comprising contacting the human neuron or nerve with a composition according to any one of claims 1-54.

76. The method according to any one of claims 73-75, comprising administering the composition to a human subject in need thereof.

77. The method according to any one of claims 73-76, comprising administering the composition by systemic intravenous injection.

78. The method according to any one of claims 73-77, comprising administering the composition prior to a surgical procedure.

79. The method according to claim 78, wherein the surgical procedure is a cancer surgical procedure.

80. The method according to claim 79, wherein the surgical procedure is a prostate cancer surgical procedure.
